(19) United States
(12) Patent Application Publication
(10) Pub. No.: US 2026/0131019 A1
(43) Pub. Date: May 14, 2026

(54) INHALABLE TARGETED GOLD NANOPARTICLE FOR ACCELERATED LUNG DELIVERY AND TREATING LUNG INFLAMMATION

(71) Applicant: The Chinese University of Hong Kong, Hong Kong (CN)

(72) Inventors: Chung Hang Jonathan CHOI, Hong Kong (CN); Shaorui LIU, Hong Kong (CN)

(21) Appl. No.: 19/386,767

(22) Filed: Nov. 12, 2025

Related U.S. Application Data
(60) Provisional application No. 63/719,519, filed on Nov. 12, 2024.

(51) Int. Cl. A61K 47/69; A61K 9/00; A61K 38/00; A61K 47/60; A61P 11/00; B82Y 5/00; C07K 14/005; C12N 1/20; C12N 15/70; C12R 1/19

(52) U.S. Cl. CPC A61K 47/6929; A61K 9/0073; A61K 47/60; A61P 11/00; C07K 14/005; C12N 1/20; C12N 15/70; A61K 38/00; B82Y 5/00; C07K 2319/21; C12N 2770/20022; C12N 2770/20033; C12R 2001/19

(57) ABSTRACT

A method for preparing inhalable targeted gold nanoparticles for accelerated lung delivery and treating lung inflammation is provided. The method includes preparing plasmids; performing plasmids transformation; performing protein expression induction; performing protein purification; and preparing spike RBD-conjugated gold nanoparticles (Au@PEG-RBD NPs). The airborne NP preferentially accumulates in lungs with lipopolysaccharides-induced ARDS to healthy lungs and enters alveolar epithelial cells that express angiotensin converting enzyme (ACE) 2 and L-SIGN, both Spike receptors activated in ARDS hamsters. The NP treats tissue injury, oxidative stress, and inflammation more effectively than corticosteroids, without gold retention in major organs or toxicity 1-year post-inhalation. The gold core blocks p38α mitogen-activated protein kinase and polo-like kinase 3. Similar efficacy is observed in hamsters with hydrochloric acid-induced ARDS. The self-therapeutic NPs, when combined with bioinspiration and non-invasive delivery, provide a safe, effective, and targeted treatment for lung inflammation.

### CROSS-REFERENCE TO RELATED APPLICATION

The present application claims the benefit of U.S. Provisional Application Ser. No. 63/719,519, filed Nov. 12, 2024, which is hereby incorporated by reference herein in its entirety, including any figures, tables, or drawings.

### SEQUENCE LISTING

The Sequence Listing for this application is labeled “CUHK-288XC1-SeqList.xml” which was created on Oct. 31, 2025 and is 4,885 bytes. The entire contents of the sequence listing is incorporated herein by reference in its entirety.

### BACKGROUND OF THE INVENTION

Acute respiratory distress syndrome (ARDS) is a leading cause of death, presenting in about 10% of patients in intensive care units and with a mortality rate of 40% worldwide^{[1]} Breakage of the air-blood barrier (ABB), a direct pathophysiological cause of ARDS^{[2]}, leads to biofluid leakage to the lung tissue and disrupted oxygen diffusion to the bloodstream. Severe inflammatory symptoms can occur within 12 hours of exposure to bacteria or chemical stimuli^{[3]}. Thus, timely management will improve treatment outcomes^{[4]}. However, conventional treatments require 5-7 days to achieve clinical response and do not target the disease cause^{[5,6]} such as invasive mechanical ventilation and anti-inflammatory drugs with side effects^{[7]}.

Nanoparticles (NPs) are under investigation for ARDS management^{[8]}. Past lung nanomedicines mostly adopted intravenous delivery to the lung endothelium (see Table 1 and Table 2) because the lung is highly vascularized. Yet, with the carrier, drugs, and targeting ligands all included, they were often large and prone to liver clearance that may cause systemic toxicity. Lung-specific delivery is possible, but it requires specialized lipid engineering^{[9]}. Importantly, intravenous delivery does not guarantee delivery to the lung epithelium, a key structural component of the ABB protecting the lungs^{[10]}, regulating ARDS-linked immune response^{[11]}, and modulating tissue repair^{[12]}. Other NPs used intratracheal instillation for local delivery to the lung epithelium (Table 2). However, instillation may elevate airway pressure and cause cardiac arrest^{[13]}, and the instilled NPs only accumulate in a confined tissue region. Intriguingly, inhalation offers rapid, noninvasive lung delivery and widespread tissue distribution^{[14]} yet seldom utilized until recently^{[15]} (Table 1). It is hypothesized that inhalable NPs that target ARDS lung epithelium may support a rapid, safe, and effective treatment.

Currently, there are four main types of treatments for ARDS, namely mechanical ventilation, corticosteroids, anti-inflammation small molecule drugs, and NPs. Among them, mechanical ventilation is invasive to the trachea and only offers symptomatic relief. Moreover, surgical opening of trachea may cause local infection, while inhalable NP is not invasive. Corticosteroids yield strong anti-inflammation efficacy, although they have severe systemic side effects and do not treat ARDS specifically. Anti-inflammation small molecule drugs (e.g., statins and ACE inhibitors) are widely tested in ARDS human trials (not approved yet), but statins have no protective effect on ARDS while ACE inhibitors only yield mild efficacy. NP-based therapies are relatively safer than previously mentioned methods. There are several types of frequently used NPs for carrying ARDS drugs to the lungs, such as liposomes, polymers NPs, and exosome-derived NPs. Nevertheless, liposomes require complicated fabrication procedures, polymer NPs have poor drug loading efficiency, while exosome-derived NPs are more difficult to be manufactured and the yield are very low.

Furthermore, current NP delivery routes have limited efficacies, particular with (1) intravenous (i.v.) or intraperitoneal injection (i.p.) and (2) intratracheal instillation (i.t.). Intravenous or intraperitoneal injection provide systemic delivery. To maximize the delivery efficiency to the lungs, targeting ligands are usually used, resulting in that most of the injected NPs accumulate in the liver and spleen. Intratracheal instillation is invasive and risky, potentially elevating airway pressure and causing cardiac arrest. Additionally, the instilled NPs (in liquid) often accumulate in a small tissue region due to inertial impaction.

### BRIEF SUMMARY OF THE INVENTION

There continues to be a need in the art for improved designs and techniques for nanoparticle-based drug delivery methods targeting the lungs.

According to an embodiment of the subject invention, a method for preparing inhalable targeted gold nanoparticles for accelerated lung delivery and treating lung inflammation is provided. The method comprises preparing plasmids; performing plasmids transformation; performing protein expression induction; performing protein purification; and preparing spike RBD-conjugated gold nanoparticles (Au@PEG-RBD NPs). The preparing plasmids comprises mixing DH5α competent cells with the plasmids; incubating the mixture on ice; heat-shocking the mixture; incubating the mixture on ice; adding pre-warmed lysogeny broth to the mixture; and incubating cells of the mixture with orbital shaking; spreading the transformed cells onto an LB-agar plate containing ampicillin and incubating the cells; selecting one colony of the cells and growing the selected colony in LB/ampicillin under shaking; collecting the cells by centrifugation and purifying the plasmids to obtain plasmids encoding His-tagged RBD (pET11a-RBD-8×His). In addition, the performing plasmids transformation comprises thawing competent cells previously stored at −80° C. on ice; adding the plasmids to the competent cells to obtain a mixture; keeping cells of the mixture on ice; heat-shocking the cells of the mixture in a water bath and incubating the cells again on ice; and adding fresh LB medium and shaking the mixture; centrifuging the cells of the mixture and discarding supernatant; resuspending remaining pelleted cells; seeding bacteria onto an LB agar plate with antibiotics and incubating it. Moreover, the performing protein expression induction comprises transforming expression strain Origami B cells, which is pre-transformed with molecular chaperone plasmid pG-KJE8, by RBD expressing plasmid pET11a-RBD; culturing the resultant on a LB agar plate; selecting one single colony of the resultant; adding the colony into fresh LB medium with antibiotics; adding overnight cultures into the fresh LB medium; and culturing the resultant until OD₆₀₀ reaches about 0.5; performing induction by adding induction buffer containing isopropyl β-D-1-thiogalactopyranoside and L-arabinose; collecting the cells by centrifugation for purification or storage at −80° C. Furthermore, the performing protein purification comprises resuspending the origami B cells in Protein Purification Buffer and lysing it by an ultrasonication processor; centrifuging the lysate; collecting supernatant of the lysate; filtering it by a syringe filter; incubating it with Ni-NTA resin; and transferring it together with the resin to a blank gravity chromatography column; discarding liquid flow-through from the lysate-resin mixture; and adding Washing Buffer to elute nonspecific binding proteins; eluting the RBD protein product by adding Elute Buffer and dialyzing it against Storage Buffer; determining purity and concentration of the protein product by sodium dodecyl sulfate (SDS)-polyacrylamide gel electrophoresis (PAGE) and Bradford assay, respectively. The preparing spike RBD-conjugated gold nanoparticles (Au@PEG-RBD NPs) comprises synthesizing citrate-capped gold NPs (cit-AuNPs) having a diameter of about 20 nm; after bringing HAuCl₄ to a boil state, adding sodium citrate under vigorous stirring and keeping the mixture boiling; cooling the resultant down to obtain cit-AuNPs; adding freshly dissolved thiol (HS)-PEG₂₀ₖ-methoxy or HS-PEG₂₀ₖ-nitrilotriacetic acid (NTA) (Biochempeg), at a 1:1 molar ratio, to the cit-AuNPs solution at a total concentration of 5 PEG molecules per nm² of the NP surface; stirring the mixture and adding NiCl₂ and stirring the mixture to obtain Au@PEG-NTA-Ni²⁺ NPs; adding His-tagged RBD proteins to the Au@PEG-NTA-Ni²⁺ NP solution under stirring to obtain Au@PEG-RBD NPS; and dialyzing the obtained NPs against Nanopure water by centrifugal filtration.

### BRIEF DESCRIPTION OF THE DRAWINGS

FIGS. 1A-1H show spike receptor-binding domain (RBD)-coated, polyethylene glycol (PEG)-stabilized gold nanoparticles (Au@PEG-RBD NPs). FIGS. 1A and 1B show expression levels of ACE2 and L-SIGN receptors in the lungs of Syrian hamsters with LPS-induced ARDS. Left: Lung tissues immunostained with L-SIGN or ACE2 (brown) at different time points post-ARDS induction. Representative images from 3 tissue sections from each hamster, n=3 hamster/group. Right: Time-dependent expression levels of ACE2 and L-SIGN in ARDS lungs by ELISA. n=3 per group, across 3 experiments. FIG. 1C shows synthesis route of Au@PEG-RBD NP. ˜20-nm, citrate-capped gold NPs were reacted with thiolated-PEG-NTA, loaded with Ni²⁺ ions, and conjugated with His-tagged Spike RBD proteins. Au@PEG-methoxy NP serves as an untargeted control. In FIG. 1D, healthy hamsters showed no obvious immunogenicity 28 days post-inhalation of Au@PEG-RBD NP (red). Intranasally instilled mammalian RBD protein served as control (blue). BALF IgA and serum IgG levels denote humoral immune and mucosal immune responses, respectively. n=3 per group, across 3 experiments. FIG. 1E shows In vitro association of Au@PEG-methoxy and Au@PEG-RBD NPs to HEK293 cells with normal or overexpressed levels of ACE2 or L-SIGN receptors 1 h post-incubation. n=3 per group, across 3 experiments. Statistical significance was calculated by Student's/test. FIG. 1F shows representative confocal images of HEK293 cells incubated with Cy5.5-labeled Au@PEG-methoxy and Au@PEG-RBD NPs (red) for 1 h. Au@PEG-RBD NPs preferentially associated with HEK293 cells that overexpress L-SIGN or ACE2 (green). Blue=nucleus. FIG. 1G shows Ex vivo association of Au@PEG-methoxy and Au@PEG-RBD NPs to healthy hamster lung tissues 0.5 hour post-intratracheal instillation. n=3 per group, across 3 experiments. Statistical significance was calculated by Student's/test. FIG. 1H shows confocal images of lung tissues of healthy hamsters that were intratracheally instilled with Au@PEG-methoxy and Au@PEG-RBD NPs (green) for 0.5 hour. Au@PEG-RBD NPs bound to ACE2 (brown) on cells ex vivo. Representative images from 3 tissue sections from each hamster, n=3 hamster/group. In FIGS. 1B, 1D, 1E, and 1G, data are presented as mean±SEM. ns, no significance; P>0.05; *P<0.05; **P<0.01.

FIGS. 2A-2F show in vivo intrapulmonary distribution of Au@PEG-RBD NPs upon inhalation by LPS-induced ARDS hamsters. FIG. 2A shows ARDS hamsters received airborne exposure of NPs for 2 h, followed by return to the holding space to await the next inhalation in 10 h or immediate sacrifice. FIG. 2B shows amounts of Au@PEG-methoxy (blue) and Au@PEG-RBD (red) NPs in the lungs of healthy and ARDS hamsters as a function of number of inhalations. Data are presented as mean±SEM. n=3 per group, across 3 experiments. Statistical significance for comparing both NP types was calculated by Student's/test. FIG. 2C shows tissue-level distribution of NPs in BALF supernatant (green), BALF cells (red), and lavaged lung tissues (blue) in healthy hamsters (left) and ARDS hamsters (right). Data are presented as mean±SEM. n=3 per group, across 3 experiments. In FIG. 2D, confocal reflectance images of lung tissues of ARDS hamsters showed widespread distribution of in lung lobes (low magnification view) and alveolus (high magnification view) of Au@PEG-RBD NPs (green; silver-enhanced) to epithelial cells (brown: E-cadherin). Black number indicates the Pearson correlation coefficient (PCC) between gold NP (green) and the cell type (brown). Representative images from 3 tissue sections from each hamster, n=3 hamsters/group. In FIG. 2E, confocal reflectance images of lung tissues of ARDS hamsters showed strong association of Au@PEG-RBD NPs (green; silver-enhanced) to L-SIGN- and ACE2-expressing cells (brown). Black number indicates the PCC between gold NP (green) and receptor type (brown). Representative images from 3 tissue sections from each hamster, n=3 hamsters/group. FIG. 2F shows that intranasally instilling antibodies against L-SIGN or ACE2 into ARDS hamsters reduced the lung accumulation of inhaled Au@PEG-RBD NP (purple), not Au@PEG-methoxy NP (green). Data are presented as mean±SEM. n=3 per group, across 1 experiment. In FIG. 2B and FIG. 2F, ns represents no significance; P>0.05; *P<0.05; **P<0.01.

FIGS. 3A-3F show efficacy of Au@PEG-RBD NP in LPS-induced ARDS hamsters. In FIG. 3A, ELISA showed that Au@PEG-RBD NP (red) reduced proinflammatory cytokines and increased anti-inflammatory cytokines in BALF supernatant more effectively than Au@PEG-methoxy NP (blue) and hydrocortisone (brown). In FIG. 3B, western blot showed that Au@PEG-RBD NP reduced proinflammatory cytokines and increased anti-inflammatory cytokines in the lavaged lung tissue more effectively than Au@PEG-methoxy NP and hydrocortisone. FIG. 3C that Au@PEG-RBD NP (red) reduced ROS levels in BALF cells more effectively than Au@PEG-methoxy NP (blue) and hydrocortisone (brown). Statistical significance was evaluated using one-way ANOVA with Tukey's post hoc test for multiple comparison. In FIG. 3D, Histological images with lung tissues damage scoring showed that Au@PEG-RBD NP reduced tissue damage more effectively than Au@PEG-methoxy NP and hydrocortisone. Statistical significance was evaluated using one-way ANOVA with Tukey's Test for post hoc analysis. Representative images from 3 tissue sections from each hamster, n=9 hamster/group. FIG. 3E shows that blocking with antibodies against L-SIGN reversed the efficacy of Au@PEG-RBD NP to a similar level of Au@PEG-methoxy NP. Data are presented as mean±SEM. n=6 per group, across 3 experiments. ns, no significance; P>0.05; **P<0.01; ***P<0.001; ****P<0.0001. In FIG. 3F, whole body plethysmography showed the restoration of key lung function parameters back to healthy levels after 3 inhalations of Au@PEG-RBD NPs. In FIGS. 3A and 3C, data are presented as mean±SEM. n=9 per group, across 3 experiments. ns, no significance; P>0.05; *P<0.05; **P<0.01; ***P<0.001; ****P<0.0001.

FIGS. 4A-4I show mechanism for the anti-ARDS efficacy of gold NPs in LPS-induced ARDS hamsters. In FIG. 4A, profiling 250 kinases by the Z′-LYTE™ assay revealed that 0.2 nM Au@PEG-methoxy NPs inhibited only 5 kinases by >70%, only MAPK14 (p38α) and PLK3 with reported linkage to ARDS. FIG. 4B shows 10-point titration curves and IC₅₀ values of Au@PEG-methox NP on MAPK14 (p38α) and PLK3. In FIG. 4C, (Left) Western blot analysis revealed that Au@PEG-RBD NP and, to a lesser extent, Au@PEG-methoxy NP inhibited p-p38α and PLK3 in ARDS hamsters when compared to untreated ARDS hamsters, in the lavaged lung tissues (top) and BALF cells (bottom). n=3, across 1 experiment. (Right) Quantification of the western blot data. Statistical significance was evaluated using one-way ANOVA with Tukey's post hoc test for multiple comparison. In FIG. 4D, western blot analysis of the lavaged lung tissue and In FIG. 4E, ELISA of BALF supernatant showed that anti-inflammatory efficacy of inhaled Au@PEG-methoxy NPs were abrogated by anisomycin, an activator of p38α. Data are presented as mean±SEM. n=9 per group, across 3 experiments. Statistical significance was evaluated using one-way ANOVA. In FIG. 4F, Volcano plot shows the distribution of differential expressed proteins (DEPs) from the pairwise comparison of “Au@PEG-methoxy NP” to “untreated” groups. n=3 (absolute fold change >2; p<0.05). Of the 2487 proteins tested, only 4 DEPs were upregulated (red) and 22 DEPs downregulated (blue). FIG. 4G shows top 5 DEPs and their expression difference and p values. In FIG. 4H, western blot analysis of LPS-induced BEAS-2B cells showed that Au@PEG-methoxy NP reduced the total expression of Elavl1 but vx-702, a p38α inhibitor, only affected the nucleus-to-cytosol translocation of Elavl1 in vitro. Au@PEG-methoxy NP could inhibit Elavl1 independent of p38α. FIG. 4I shows quantification of the western blot data of FIG. 4H. Data are presented as mean±SEM. n=3 per group, across 3 experiments. Statistical significance was evaluated using one-way ANOVA. ns, no significance; P>0.05; *P<0.05; ***P<0.001; ****P<0.0001.

FIGS. 5A-5C show long-term toxicity of Au@PEG-RBD NP upon three inhalations by LPS-induced ARDS hamsters. FIG. 5A shows organ-level distribution of Au@PEG-RBD (red) and Au@PEG-methoxy (blue) NP. No gold was detected in various major internal organs 1 y post-inhalation, indicating clearance of gold NPs. Data are from n=3, across 1 experiment. Histological images of FIG. 5B (lungs) and of FIG. 5C (other major internal organs) showed no abnormal tissue morphology 1 y post-inhalation of Au@PEG-RBD NP when compared to healthy control. Representative images from 1 tissue section from each hamster, n=3 hamster/group.

FIGS. 6A-6E show efficacy of Au@PEG-RBD NP in HCl-induced ARDS hamsters. FIG. 6A shows expression of ACE2 and L-SIGN receptors in the lungs of Syrian hamsters with HCl-induced ARDS. Left: Western blot of lung tissues blotted with L-SIGN or ACE2 at various time points post-ARDS induction. n=3 hamster/group. Middle: Time-dependent expression of ACE2 and L-SIGN in ARDS lungs by ELISA. n=3 per group, across 3 experiments. Right: Lung tissues immunostained with L-SIGN or ACE2 (brown) at different time points post-ARDS induction. Representative images from 3 tissue sections from each hamster, n=3 hamster/group. In FIG. 6B, ELISA showed that Au@PEG-RBD NP (red) reduced proinflammatory cytokines and increased anti-inflammatory cytokines in BALF supernatant more effectively than Au@PEG-methoxy NP (blue) and hydrocortisone (brown). In FIG. 6C, western blot showed that Au@PEG-RBD NP reduced proinflammatory cytokines in lavaged lung tissues most effectively. FIG. 6D showed that Au@PEG-RBD NP (red) reduced ROS levels in BALF cells more effectively than Au@PEG-methoxy NP (blue) and hydrocortisone (brown). Statistical significance was evaluated using one-way ANOVA with Tukey's post hoc test for multiple comparison. In FIG. 6B and FIG. 6D, data are presented as mean±SEM. n=9 per group, across 3 experiments. FIG. 6E shows that blocking with antibodies against L-SIGN reversed the efficacy of Au@PEG-RBD NP to a similar level of Au@PEG-methoxy NP. Data are presented as mean±SEM. n=6 per group, across 3 experiments. ns, no significance; P>0.05; *P<0.05; **P<0.01; ***P<0.001; ****P<0.0001.

FIG. 7 shows expression level of ACE2 (brown) in the lungs of ARDS hamsters at various time points post-LPS induction. The ACE2 signals were the most intense at 36 h post-induction. Representative images from 7 tissue sections from each hamster, n=3 hamster/group.

FIG. 8 shows expression levels of L-SIGN (CD299; brown) in the lungs of ARDS hamsters at various time points post-LPS induction. L-SIGN signals were the most intense at 12 h post-induction. Representative images from 7 tissue sections from each hamster, n=3 hamster/group.

FIG. 9 shows expression levels of mannose receptor (CD206; brown) in the lungs of ARDS hamsters at various time points post-LPS induction. Mannose receptor signal was low within 72 h post-induction. Representative images from 7 tissue sections from each hamster, n=3 hamster/group.

FIG. 10 shows expression levels of DC-SIGN (CD209; brown) in the lungs of ARDS hamsters at various time points post-LPS induction. DC-SIGN signal was low within 72 h post-induction. Representative images from 7 tissue sections from each hamster, n=3 hamsters/group.

FIG. 11 shows expression levels of MGL (CD301; brown) in the lungs of ARDS hamsters at various time points post-LPS induction. MGL signal was low within 72 h post-induction. Representative images from 7 tissue sections from each hamster, n=3 hamsters/group.

FIG. 12 shows expression levels of TMPRSS2 (brown) in the lungs of ARDS hamsters at various time points post-LPS induction. TMPRSS2 signal was low within 72 h post-induction. Representative images from 7 tissue sections from each hamster, n=3 hamsters/group.

FIGS. 13A-13E show expression and purification of Spike RBD protein (with a C terminus 8×Histag). FIG. 13A shows that of the three strains of E. Coli tested, western blot analysis indicated that the expression of soluble Spike RBD protein was the highest in Origami B (right), as evidenced by thick bands in the soluble fraction (supernatant of lysate) due to immunostaining of Spike RBD and its His tag. FIGS. 13B and 13C show that of the five plasmids of folding chaperones tested, co-expression of Spike RBD plasmid (pET11a-RBD-8×His) in Origami B cells with pG-KJE8 resulted in the most abundant RBD expression and strongest binding to ACE2 and L-SIGN receptors [or lowest dissociation constant (K_{d})]. FIG. 13D shows stepwise protein detection during purification. All elute fractions were collected for centrifugal filtration (MWCO 30 kDa) for 3 times at 4° C. for later use. FIG. 13E shows further purification of Spike RBD protein [300 and 500 mM imidazole elute in (FIG. 13D)] using a 30 kDa MWCO membrane removed other impurity bands; we collected both elute fractions of RBD protein. The expected molecular weight of Spike RBD protein is 25 kDa.

FIG. 14 shows validation of the conjugation of Spike RBD proteins on the Au@PEG NP. Initially, Spike RBD proteins (with a His tag) were conjugated to the NTA group of Au@PEG-NTA-Ni²⁺ NP, thus forming Au@PEG-RBD NPs. After removing the unreacted Spike RBD proteins by centrifugal filter and washing the NPs with Tris buffer, imidazole was added to the purified Au@PEG-RBD NPs to release the bound Spike RBD proteins. Lastly, western blot analysis confirmed the release of Spike RBD proteins from the Au@PEG NP, as evidenced by the protein bands following separate immunoblotting of Spike RBD and its His tag. Addition of free imidazole breaks the interaction between the nickel nitrilotriacetic acid (Ni-NTA) on the PEG strand and histidine tag of the Spike protein. By ELISA of the released Spike RBD proteins (Component 3), the number of RBD proteins on the gold NP was ˜306.

FIG. 15 shows batch-to-batch consistency of as-synthesized Au@PEG-methoxy and Au@PEG-RBD NPs, as evidenced by hydrodynamic size distribution histograms of three representative batches was shown. Each batch was synthesized at an interval of 2-3 months.

FIG. 16 shows stability of Au@PEG-RBD NP as a function of Spike RBD loading. Au@PEG-RBD NPs with 100% PEG strands loaded with Spike RBD faced agglomeration 24 h post-incubation in BALF (possibly due to protein-protein interaction), but such agglomeration was readily reversible by sonication. Conversely, Au@PEG-RBD NPs with only 50% of PEG strands loaded with Spike RBD remained stable in BALF for 3 d. For our subsequent studies, we choose 50% RBD as the default loading for constructing the Au@PEG-RBD NP.

FIG. 17 shows representative TEM images of citrate-capped gold, Au@PEG-methoxy, and Au@PEG-RBD NPs with negative stain by EMStainer. The PEG shell appears as a light halo around the gold core because of its lower electron scattering power relative to the gold core and the surrounding heavy-metal background stain. Physical diameter of NPs (as measured by TEM by manually counting 500 NPs per group) were smaller than hydrodynamic diameter (as measured by DLS) because TEM images were captured under dry condition.

FIG. 18 shows batch-to-batch consistency of aerosolized Au@PEG-methoxy and Au@PEG-RBD NPs, as evidenced by aerodynamic size distribution histograms and aerosolized NP concentration of three representative batches.

FIGS. 19A-19C show stability of Au@PEG-RBD NPs in BALF. FIGS. 19A and 19B show that when compared to incubation in water, UV-vis spectrophotometry did not reveal a drastic change in the surface plasmon resonance (SPR) peaks of Au@PEG-RBD NPs upon incubation in BALF at 37° C. for 24 h; the SPR peaks remained at 524 nm. In FIG. 19C, DLS measurements showed that the hydrodynamic size of both NPs increased by ˜20 nm upon BALF incubation, but there was no obvious difference in hydrodynamic size between Au@PEG-methoxy and Au@PEG-RBD NPs. Data are presented as mean±SEM. n=3 per group, across 3 experiments. Statistical significance was calculated by One-way ANOVA. *P<0.05; **P<0.01.

FIGS. 20A-20C show ligand stability of Au@PEG-RBD NP. FIG. 20A show that after incubating the NPs with fresh BALF extracted from hamsters at 37° C. for 48 h, the supernatant was subjected to western blot (Lane #1). Next, NPs were resuspended in 1 M imidazole to release the Spike RBD proteins originally conjugated to the gold core. The supernatant was subjected to western blot (Lane #2) or ELISA. In FIG. 20B, western blot showed that, after BALF incubation, almost all RBD proteins remained on the gold core. In FIG. 20C, ELISA indicated that the total number of RBD proteins on the Au@PEG-RBD NP did not change after BALF incubation. These data suggest the stability of Au@PEG-RBD NP in BALF, with limited shedding of targeting ligands from the NP.

FIGS. 21A-21B show binding of Au@PEG-RBD NPs to Spike receptors. FIG. 21A shows binding affinity of Au@PEG-RBD NPs to ACE2 or L-SIGN receptors as measured by our in-house modified ELISA assay^{[63]}. Au@PEG-RBD NPs bound to ACE2 (red) and L-SIGN (orange) at K_{d} values of 0.23 and 0.83 nM, respectively. Au@PEG-methoxy NPs did not appreciably bind to ACE2 (dark blue) or L-SIGN (light blue). Data are presented as mean±SEM. n=6 per group, across 3 experiments. FIG. 21B shows Ligand competition assay of Au@PEG-RBD NPs to ACE2 or L-SIGN as measured by our in-house modified ELISA assay. In each group, the concentration of Au@PEG-RBD NPs fixed at 3 nM while the concentration of free RBD increased gradually. Data are presented as mean±SEM. n=6 per group, across 3 experiments.

FIG. 22 shows leakage of nickel ions from Au@PEG-RBD NPs. Upper part shows a schematic diagram of detection flow. In this work, to minimize the nickel exposure, Au@PEG-RBD NP solutions were dialyzed against Nanopure water for 6 times before inhalation. ICP-MS is used to detect nickel ion concentration in supernatant after dialysis and 1-month storage. Data are presented as mean±SEM. Data are from n=3, across 2 experiments. Statistical significance was calculated by student's/test. No significance (ns), P>0.05.

FIG. 23 shows cytotoxicity of Au@PEG-methoxy and Au@PEG-RBD NPs. Pre-seeded in 96-well plates, A549, BEAS-2B, and HEK293 cells were incubated in medium containing different concentrations of Au@PEG-methoxy or Au@PEG-RBD NPs for 72 h. The medium used for A549 and HEK293 cells was complete DMEM, and the medium for BEAS-2B cells was LHC-8. The alamarBlue reagent (Invitrogen) was used to test cell viability by measuring the optical absorbance at 570 nm and 600 nm by a Multiskan GO UV-absorbance microplate reader. Data are represented as mean±SD. n=6 per group, across 2 experiments. We choose 0.2 nM as the incubation concentration for our in vitro cellular uptake studies.

FIG. 24 shows time-dependent cellular association of Au@PEG-RBD NP to HEK293 cells with L-SIGN and ACE2 overexpression. Au@PEG-RBD NP associated with ACE2- and L-SIGN-overexpressing cells more abundantly than Au@PEG-methoxy NP over shorter incubation periods. Over longer incubation periods, Au@PEG-RBD NP associated with both types of overexpressing cells at similar amounts to Au@PEG-methoxy NP. Data are presented as mean±SEM. Data are from n=3, across 2 experiments. Statistical significance was calculated by Two-way ANOVA. **P<0.001, *P<0.05, No significance (ns), P>0.05.

FIG. 25 shows additional confocal images of HEK293 cells incubated with Cy5.5-labeled Au@PEG-RBD NPs (red) for 1 h. Au@PEG-RBD NPs preferentially associated with HEK293 cells that overexpress ACE2 (green). Blue=nucleus. White number indicates the PCC between gold NP (red) and receptor (green). A similar image was shown in FIG. 1F.

FIG. 26 shows additional confocal images of HEK293 cells incubated with Cy5.5-labeled Au@PEG-RBD NPs (red) for 1 h. Au@PEG-RBD NPs preferentially associated with HEK293 cells that overexpress L-SIGN (green). Blue=nucleus. White number indicates the PCC between gold NP (red) and receptor (green). A similar image was shown in FIG. 1F.

FIGS. 27A-27B show confocal images of HEK293 cells, with varying levels of (FIG. 27A) ACE2 and (FIG. 27B) L-SIGN expression (green), incubated with Cy5.5-labeled Au@PEG-RBD NPs (red) for 1 h. NPs preferentially entered cells overexpressing ACE2/L-SIGN by overlapping with receptors. Blue=nucleus. White number indicates the PCC between gold NP (red) and receptor (green).

FIG. 28 shows additional confocal images of HEK293 cells with low ACE2 expression (green) incubated with Cy5.5-labeled Au@PEG-RBD NPs (red) for 1 h. Amount of cellular entry and colocalization with ACE2 were lower than those in ACE2-overexpressing cells. Blue=nucleus. White number indicates the PCC between gold NP (red) and receptor (green).

FIG. 29 shows additional confocal images of HEK293 cells with low L-SIGN expression (green) incubated with Cy5.5-labeled Au@PEG-RBD NPs (red) for 1 h. Amount of cellular entry and colocalization with L-SIGN were lower than those in L-SIGN-overexpressing cells. Blue=nucleus. White number indicates the PCC between gold NP (red) and receptor (green).

FIG. 30 shows calculation of maximum nickel exposure in hamsters. We assume two forms of Ni²⁺ ions present in the aqueous NP solution prior to aerosolization, (1) free ions in water and (2) chelated ions in the NP. The amount of free Ni²⁺ in water was measured directly by ICP-MS (FIG. 22), whereas the amounts of chelated Ni²⁺ was inferred stoichiometrically based on the inhaled gold contents in the lungs as measured by ICP-MS in FIG. 2B (˜300 Ni²⁺ ions per Au@PEG-RBD NP). In total, ˜12 μg of nickel (free and chelated) was inhaled by hamsters after three inhalations that spanned the course of 36-h treatment, lower than the theoretical tolerable upper intake level. Based on the Tolerable Upper Intake Level for Nickel for humans (1 mg/day^{[68]}), we used the FDA equivalent dose^{[24]} to estimate a tolerable upper limit dose for hamsters (18.5 μg/150-g-hamster/day). In fact, the actual uptake of free nickel ions was even less than our theoretical calculation owing to the clearance of aerosolized water droplets by the drier prior to transport to the animal inhalation chamber.

FIG. 31 shows organ-level (left) and upper/lower airways (right) distribution of Au@PEG-RBD (triangle) and Au@PEG-methoxy (square) NPs in healthy hamsters upon three inhalations, based on the timeline in FIG. 2A. Data are presented as mean±SEM. Data are from n=3, across 1 experiment. Statistical significance was calculated by two-way ANOVA. No significance (ns), P>0.05.

FIG. 32 shows organ-level (left) and upper/lower airways (right) distribution of Au@PEG-RBD (triangle) and Au@PEG-methoxy NPs (square) in LPS-induced ARDS hamsters upon three inhalations, based on the timeline in FIG. 2A. Data are presented as mean±SEM. Data are from n=3, across 3 experiments. Statistical significance was calculated by two-way ANOVA. No significance (ns), P>0.05.

FIG. 33 shows tissue-level distribution of Au@PEG-RBD and Au@PEG-methoxy NPs in the different lung lobes of both healthy and LPS-induced ARDS hamsters, following the timeline in FIG. 2A. Data are presented as mean±SEM. Data are from n=3, across 2 experiments. Statistical analysis was determined by two-way ANOVA. No significance (ns), P>0.05.

FIG. 34 shows amount of Au@PEG-methoxy (square) and Au@PEG-RBD (triangle) NPs in the lungs of healthy and ARDS hamsters as a function of number of inhalations. The plots of Inhalation 3 are identical to the corresponding plot in FIG. 2B. Data are presented as mean±SEM. n=3 per group, across 1 experiment. Statistical significance for comparing both NP types was calculated by Student's t test. *P<0.05; **P<0.001.

FIG. 35 shows confocal reflectance images of lung tissues of ARDS hamsters showed weak association of Au@PEG-RBD NPs (green; silver-enhanced) to endothelial cells (brown: CDH5). Black number indicates the Pearson correlation coefficient (PCC) between gold NP (green) and the cell type (brown). Representative images from 3 tissue sections from each hamster, n=3 hamsters/group.

FIG. 36 shows specific binding of Au@PEG-RBD NPs to L-SIGN and ACE2 receptors in ARDS lungs. Pretreatment via intratracheal administration of antibodies against L-SIGN reduced the accumulation of Au@PEG-RBD NP in the lungs of LPS-induced ARDS hamsters after the first two inhalations, not after the third inhalation. By contrast, pretreatment via intratracheal administration of antibodies against ACE2 did not reduce the accumulation of Au@PEG-RBD NP after the first two inhalations, but it did block lung accumulation after the third inhalation. The data of “Au@PEG-methoxy”, “Au@PEG-RBD”, “anti-L-SIGN antibody+Au@PEG-RBD”, “anti-ACE2 antibody+Au@PEG-RBD”, and “anti-L-SIGN antibodies+anti-L-SIGN antibodies+Au@PEG-RBD NPs.” groups are identical to the corresponding group in FIG. 2F. Data are presented as mean±SEM. n=3 per group, across 3 experiments. Statistical analysis was determined by one-way ANOVA. No significance (ns), P>0.05; *P<0.05; **P<0.001; ***P<0.001.

FIG. 37 shows combined roles of L-SIGN and ACE2 receptors in mediating the delivery of Au@PEG-RBD NP to ARDS lungs. LPS-induced ARDS hamsters inhaled only one dose of NPs 12 h (1^{st} time point; top panel) or 36 h (3^{rd} time point; bottom panel) post-LPS induction. Antibodies against L-SIGN, antibodies against ACE2, or both antibodies were intratracheally administered 1 h before NP inhalation. Results in the top panel were identical to those for the first inhalation time point as shown in FIG. 2f. Data are presented as mean #SEM. n=3 per group, across 3 experiments.

FIGS. 38A-38C show protein corona analysis of Au@PEG-methoxy and Au@PEG-RBD NPs upon incubation in BALF extracted from LPS-induced ARDS Syrian hamsters for 30 min. FIG. 38A shows top 10 BALF proteins in the protein corona. FIG. 38B shows SDS-PAGE gel analysis of protein corona from the NP. FIG. 38C shows pretreatment via antibodies against FcR or SPARC (both receptors of albumin) did not drastically reduce the accumulation of Au@PEG-RBD NPs in the lungs of ARDS hamsters. Therefore, the BALF protein corona did not affect the lung delivery of Au@PEG-RBD NP. Data are presented as mean±SEM. n=3 per group, across 3 experiments. Statistical analysis was determined by one-way ANOVA. No significance (ns), P>0.05. *P<0.05; ***P<0.001.

FIG. 39 shows inhalation plans for ARDS treatment. According to the data of FIGS. 1A, 1B, 7 and 8, L-SIGN and ACE2 upregulated at ˜12 and ˜36 h post ARDS induction, respectively. We made 2 inhalation plans of different start points to target L-SIGN (plan 1, this plan is identical to the timeline shown in FIG. 2A) and ACE2 (plan 2).

FIG. 40 shows quantification of the levels of proinflammatory cytokines (TNF-α, IFN-γ, IL-6, and IL-8) and anti-inflammatory cytokines (IL-4, and IL-10) in BALF extracted from healthy hamsters with/without Au@PEG-RBD NP treatment (following the timeline in FIG. 2A). Data are presented as mean±SEM. Data are from n=6, across 3 experiments. Statistical analysis was determined by one-way ANOVA. No significance (ns), P>0.05.

FIG. 41 shows quantification of the level of TNF-α in BALF extracted from LPS-induced ARDS hamsters where treatment started 12 h post-induction (following the timeline in FIG. 2A). The top row is identical to the corresponding plot in FIG. 3A, and the bottom row shows the detailed values of each group with statistical analysis. Data are presented as mean±SEM. Data are from n=9, across 3 experiments. Statistical analysis was determined by one-way ANOVA. No significance (ns), P>0.05; **P<0.01; ***P<0.001; *P<0.0001.

FIG. 42 shows quantification of the level of TNF-α in BALF extracted from LPS-induced ARDS hamsters where treatment started 36 h post-induction (following Inhalation plan 2 in FIG. 39). The bottom row shows the detailed values of each group of each curve in the top row with statistical analysis. Data are presented as mean±SEM. Data are from n=6, across 3 experiments. Statistical analysis was determined by one-way ANOVA. *P<0.05; **P<0.01; ***P<0.001; ****P<0.0001.

FIG. 43 shows quantification of the level of IFN-γ in BALF extracted from LPS-induced ARDS hamsters where treatment started 12 h post-induction (following the timeline in FIG. 2A). The top row is identical to the corresponding plot in FIG. 3A, and the bottom row shows the detailed values of each group with statistical analysis. Data are presented as mean±SEM. Data are from n=9, across 3 experiments. Statistical analysis was determined by one-way ANOVA. No significance (ns); ****P<0.0001.

FIG. 44 shows quantification of the level of IFN-γ in BALF extracted from LPS-induced ARDS hamsters where treatment started 36 h post-induction (following Inhalation plan 2 in FIG. 39). The bottom row shows the detailed values of each group of each curve in the top row with statistical analysis. Data are presented as mean±SEM. Data are from n=6, across 3 experiments. Statistical analysis was determined by one-way ANOVA. **P<0.01; ***P<0.001; ****P<0.0001.

FIG. 45 shows quantification of the level of IL-6 in BALF extracted from LPS-induced ARDS hamsters where treatment started 12 h post-induction (following the timeline in FIG. 2A). The top row is identical to the corresponding plot in FIG. 3A, and the bottom row shows the detailed values of each group with statistical analysis. Data are presented as mean±SEM. Data are from n=9, across 3 experiments. Statistical analysis was determined by one-way ANOVA. **P<0.01; ****P<0.0001.

FIG. 46 shows quantification of the level of IL-6 in BALF extracted from LPS-induced ARDS hamsters where treatment started 36 h post-induction (following Inhalation plan 2 in FIG. 39). The bottom row shows the detailed values of each group with statistical analysis. Data are presented as mean #SEM. Data are from n=6, across 3 experiments. Statistical analysis was determined by one-way ANOVA. **P<0.01; ****P<0.0001.

FIG. 47 shows quantification of the level of IL-8 in BALF extracted from LPS-induced ARDS hamsters where treatment started 12 h post-induction (following the timeline in FIG. 2A). The top row is identical to the corresponding plot in FIG. 3A, and the bottom row shows the detailed values of each group with statistical analysis. Data are presented as mean±SEM. Data are from n=9, across 3 experiments. Statistical analysis was determined by one-way ANOVA. No significance (ns), P>0.05; **P<0.01; ***P<0.001; ****P<0.0001.

FIG. 48 shows quantification of the level of IL-8 in BALF extracted from LPS-induced ARDS hamsters where treatment started 36 h post-induction (following Inhalation plan 2 in FIG. 39). The bottom row shows the detailed values of each group with statistical analysis. Data are presented as mean #SEM. Data are from n=6, across 3 experiments. Statistical analysis was determined by one-way ANOVA. **P<0.01; ****P<0.0001.

FIG. 49 shows quantification of the level of IL-4 in BALF extracted from LPS-induced ARDS hamsters where treatment started 12 h post-induction (following the timeline in FIG. 2A). The top row is identical to the corresponding plot in FIG. 3A, and the bottom row shows the detailed values of each group with statistical analysis. Data are presented as mean±SEM. Data are from n=9, across 3 experiments. Statistical analysis was determined by one-way ANOVA. No significance (ns), P>0.05; ***P<0.001; ****P<0.0001.

FIG. 50 shows quantification of the level of IL-10 in BALF extracted from LPS-induced ARDS hamsters where treatment started 12 h post-induction (following the timeline in FIG. 2A). The top row is identical to the corresponding plot in FIG. 3A, and the bottom row shows the detailed values of each group with statistical analysis. Data are presented as mean±SEM. Data are from n=9, across 3 experiments. Statistical analysis was determined by one-way ANOVA. No significance (ns), P>0.05; ***P<0.001; ****P<0.0001.

FIG. 51 shows quantification of the level of cytokines in BALF upon inhalation of free PEG-RBD conjugates. BALF was extracted from LPS- or HCl-induced ARDS hamsters after 3 inhalations following the timelines shown in FIG. 39. The data of Group A is identical to the Untreated group in FIGS. 41-48 and FIGS. 56-63. Data are presented as mean±SEM. Data are from n=6, across 3 experiments. Statistical analysis was determined by student's/test. No significance (ns), P>0.05.

FIG. 52 shows quantification of the western blot data shown in FIG. 3B. Data are presented as mean±SEM. Data are from n=3, across 3 experiments. Statistical significance was evaluated using two-way ANOVA. No significance (ns), P>0.05; *P<0.05; **P<0.001; ***P<0.001; ****P<0.0001.

FIG. 53 shows counting cells in BALF. Red blood cells (RBC) and other BALF cells were counted by a hemocytometer slide. The ratio of different cell types was counted by Wright-Giemsa staining. Western blot indicates that increased macrophages were mainly M2 macrophages.

FIG. 54 shows tissue repairing efficacy of inhaled Au@PEG-RBD NPs in LPS-induced ARDS hamsters after 1^{st} and 2^{nd} inhalations. Lung damage scoring (left part) [with lung tissue histological images shown in (right part)] showed that Au@PEG-RBD NP reduced tissue damage more effectively than Au@PEG-methoxy NP and hydrocortisone. Representative images from 3 tissue sections from each hamster, n=9 hamster/group. Statistical significance was evaluated using one-way ANOVA.

FIG. 55 shows quantification of the level of cytokines in BALF upon the specific antibody blocking in LPS-induced hamsters. Anti-L-SIGN antibody was intranasally injected into ARDS hamsters 2 hours prior to each inhalation. BALF was extracted from LPS-induced ARDS hamsters where treatment started 12 h post-induction (following the timeline in FIG. 2a). The data of Group A and B are identical to the corresponding groups in FIG. 41, FIG. 43, FIG. 45, FIG. 47, FIG. 49, and FIG. 50. The plots of “3rd Inhalation” are identical to the corresponding plots in FIG. 3E. Data are presented as mean±SEM. Data are from n=6, across 3 experiments. Statistical analysis was determined by one-way ANOVA. No significance (ns), P>0.05; *P<0.05; **P<0.01; ***P<0.001; ****P<0.0001.

FIG. 56 shows quantification of the level of cytokines in BALF upon the specific antibody blocking in LPS-induced hamsters. Anti-ACE2 antibody was intranasally injected into ARDS hamsters 2 hours prior to each inhalation. BALF was extracted from LPS-induced ARDS hamsters where treatment started 36 h post-induction (following Inhalation plan 2 in FIG. 39). The data of Group A and B are identical to the corresponding groups in FIG. 42, FIG. 44, FIG. 46, and FIG. 48. Data are presented as mean±SEM. Data are from n=6, across 3 experiments. Statistical analysis was determined by one-way ANOVA. No significance (ns), P>0.05; *P<0.05; **P<0.01.

FIG. 57 shows lung function parameters of healthy (left), LPS-induced ARDS (middle), and NP-treated, LPS-induced ARDS hamsters (right) measured by whole-body plethysmography. Results showed that peak inspiratory flow (PIF), peak expiratory flow (PEF), expiratory time (Te), and relaxation time (Tr) were restored to healthy levels after 3 inhalations of Au@PEG-RBD NPs. Data are presented as mean±SEM. n=5 per group, across 3 experiments. ns, no significance; P>0.05; *P<0.05; **P<0.01.

FIG. 58 shows quantification of the level of TNF-α in BALF extracted from HCl-induced ARDS hamsters where treatment started 12 h post-induction (following Inhalation plan 1 in FIG. 39). The top row is identical to the corresponding plot in FIG. 6B, and the bottom row shows the detailed values of each group with statistical analysis. Data are presented as mean±SEM. Data are from n=9, across 3 experiments. Statistical analysis was determined by one-way ANOVA. No significance (ns), P>0.05; *P<0.05; **P<0.01; ****P<0.0001.

FIG. 59 shows quantification of the level of TNF-α in BALF extracted from HCl-induced ARDS hamsters where treatment started 36 h post-induction (following Inhalation plan 2 in FIG. 39). The bottom row shows the detailed values of each group with statistical analysis. Data are presented as mean±SEM. Data are from n=6, across 3 experiments. Statistical analysis was determined by one-way ANOVA. No significance (ns), P>0.05; **P<0.01; ***P<0.001; ****P<0.0001.

FIG. 60 shows quantification of the level of IFN-γ in BALF extracted from HCl-induced ARDS hamsters where treatment started 12 h post-induction (following Inhalation plan 1 in FIG. 39). The top row is identical to the corresponding plot in FIG. 6B, and the bottom row shows the detailed values of each group with statistical analysis. Data are presented as mean±SEM. Data are from n=9, across 3 experiments. Statistical analysis was determined by one-way ANOVA. *P<0.05; ***P<0.001; ****P<0.0001.

FIG. 61 shows quantification of the level of IFN-γ in BALF extracted from HCl-induced ARDS hamsters where treatment started 36 h post-induction (following Inhalation plan 2 in FIG. 39). The bottom row shows the detailed values of each group with statistical analysis. Data are presented as mean±SEM. Data are from n=6, across 3 experiments. Statistical analysis was determined by one-way ANOVA. No significance (ns), P>0.05; *P<0.05; **P<0.01; ***P<0.001; ****P<0.0001.

FIG. 62 shows quantification of the level of IL-6 in BALF extracted from HCl-induced ARDS hamsters where treatment started 12 h post-induction (following Inhalation plan 1 in FIG. 39). The top row is identical to the corresponding plot in FIG. 6B, and the bottom row shows the detailed values of each group with statistical analysis. Data are presented as mean±SEM. Data are from n=9, across 3 experiments. Statistical analysis was determined by one-way ANOVA. No significance (ns), P>0.05; *P<0.05; ***P<0.001; ****P<0.0001.

FIG. 63 shows quantification of the level of IL-6 in BALF extracted from HCl-induced ARDS hamsters where treatment started 36 h post-induction (following Inhalation plan 2 in FIG. 39). The bottom row shows the detailed values of each group with statistical analysis. Data are presented as mean #SEM. Data are from n=6, across 3 experiments. Statistical analysis was determined by one-way ANOVA. No significance (ns), P>0.05; **P<0.01; ***P<0.001; ****P<0.0001.

FIG. 64 shows quantification of the level of IL-8 in BALF extracted from HCl-induced ARDS hamsters where treatment started 12 h post-induction (following Inhalation plan 1 in FIG. 39). The top row is identical to the corresponding plot in FIG. 6B, and the bottom row shows the detailed values of each group with statistical analysis. Data are presented as mean±SEM. Data are from n=9, across 3 experiments. Statistical analysis was determined by one-way ANOVA. No significance (ns), P>0.05; *P<0.05; **P<0.01; ***P<0.001; ****P<0.0001.

FIG. 65 shows quantification of the level of IL-8 in BALF extracted from HCl-induced ARDS hamsters where treatment started 36 h post-induction (following Inhalation plan 2 in FIG. 39). The bottom row shows the detailed values of each group with statistical analysis. Data are presented as mean #SEM. Data are from n=6, across 3 experiments. Statistical analysis was determined by one-way ANOVA. ****P<0.0001.

FIG. 66 shows quantification of the level of IL-4 in BALF extracted from HCl-induced ARDS hamsters where treatment started 12 h post-induction (following the timeline in FIG. 2A). The top row is identical to the corresponding plot in FIG. 6B, and the bottom row shows the detailed values of each group with statistical analysis. Data are presented as mean±SEM. Data are from n=9, across 3 experiments. Statistical analysis was determined by one-way ANOVA. No significance (ns), P>0.05; ***P<0.001; ****P<0.0001.

FIG. 67 shows quantification of the level of IL-10 in BALF extracted from HCl-induced ARDS hamsters where treatment started 12 h post-induction (following the timeline in FIG. 2A). The top row is identical to the corresponding plot in FIG. 6B, and the bottom row shows the detailed values of each group with statistical analysis. Data are presented as mean±SEM. Data are from n=9, across 3 experiments. Statistical analysis was determined by one-way ANOVA. No significance (ns), P>0.05; *P<0.05; ****P<0.0001.

FIG. 68 shows quantification of the western blot data shown in FIG. 6C. Data are presented as mean±SEM. Data are from n=3, across 3 experiments. Statistical significance was evaluated using two-way ANOVA. *P<0.05; **P<0.001; ***P<0.001; ****P<0.0001.

FIG. 69 shows tissue repairing efficacy of inhaled Au@PEG-RBD NPs in HCl-induced ARDS hamsters. Lung tissue histological images (upper part) [with lung damage scoring shown in (bottom part)] showed that Au@PEG-RBD NP reduced tissue damage more effectively than Au@PEG-methoxy NP and hydrocortisone. Representative images from 3 tissue sections from each hamster, n=9 hamster/group. Statistical significance was evaluated using one-way ANOVA.

FIG. 70 shows quantification of the level of cytokines in BALF upon the specific antibody blocking in HCl-induced hamsters. Anti-L-SIGN antibody was intranasally injected into ARDS hamsters 2 hours prior to each inhalation. BALF was extracted from HCl-induced ARDS hamsters where treatment started 12 h post-induction (following the timeline in FIG. 2A). The data of Group A and B are identical to the corresponding groups in FIG. 58, FIG. 60, FIG. 62, FIG. 64, FIG. 66, and FIG. 67. The plots of “3rd Inhalation” are identical to the corresponding plots in FIG. 6E. Data are presented as mean±SEM. Data are from n=6, across 3 experiments. Statistical analysis was determined by one-way ANOVA. No significance (ns), P>0.05; *P<0.05; **P<0.01; ***P<0.001; ****P<0.0001.

FIG. 71 shows quantification of the level of cytokines in BALF upon the specific antibody blocking in HCl-induced hamsters. Anti-ACE2 antibody was intranasally injected into ARDS hamsters 2 hours prior to each inhalation. BALF was extracted from HCl-induced ARDS hamsters where treatment started 36 h post-induction (following Inhalation plan 2 in FIG. 39). The data of Group A and B are identical to the corresponding groups in FIG. 59, FIG. 61, FIG. 63, and FIG. 65. Data are presented as mean±SEM. Data are from n=6, across 3 experiments. Statistical analysis was determined by one-way ANOVA. No significance (ns), P>0.05; *P<0.05; **P<0.01; ***P<0.001; ****P<0.0001.

FIG. 72 shows quantification of the ROS level in BALF cells upon the specific antibody blocking or free PEG-RBD conjugates in ARDS hamsters. Anti-L-SIGN antibody was intranasally injected into ARDS hamsters 2 hours prior to each inhalation. BALF was extracted from ARDS hamsters where treatment started 12 h post-induction (following the timeline in FIG. 2A). The data of Untreated, Au@PEG-methoxy, and Au@PEG-RBD groups are identical to the corresponding groups in FIG. 3C and FIG. 6D. Data are presented as mean±SEM. Data are from n=6, across 3 experiments. Statistical analysis was determined by one-way ANOVA. No significance (ns), P>0.05; *P<0.05.

TABLE 1
Summary of Non-gold Nanoparticles (NPs) for ARDS Management.

| | | | Animal species, | |
|---|---|---|---|---|
| | | Drug loading, | delivery route, | |
| | | Targeting ligands, | and treatment | Main therapeutic |
| Reference | NP and size | and target site | duration | outcomes |
| Chen et al. | Liposome | Dexamethasone | SD rats | Significant |
| 2013 ^{[69]} | 136 ± 38 nm | Antibody against | i.v. | dexamethasone |
| | | pulmonary | | accumulation in the |
| | | surfactant protein A | | lungs, superior |
| | | (SP-A) | | efficacy than free |
| | | Lung surfactant | Continuous daily | drug |
| | | (lung lining fluid) | injections for 2 | |
| | | | weeks after | |
| | | | disease induction | |
| Howard et | Liposome | Antioxidant | C57BL/6 mice | Decreased level of |
| al. 2014 ^{[70]} | 197.8 ± 4.5 nm | chemical EUK-134 | | vascular cell |
| | | Antibody against | i.v. | adhesion protein 1 |
| | | platelet-endothelial | | (VCAM1) and lung |
| | | cell adhesion | | edema |
| | | molecule 1 | | |
| | | (PECAM-1) | | |
| | | Endothelial cells | Injected once at | |
| | | | 15 min prior to | |
| | | | LPS induction, | |
| | | | mice lungs were | |
| | | | collected 24 h | |
| | | | after induction | |
| Wang et al. | Denatured | Piceatannol, a | CD1 mice | Increased NP |
| 2014 ^{[71]} | bovine serum | spleen tyrosine | | internalization by |
| | albumin | kinase inhibitor | | neutrophils and |
| | (BSA) NP | N.A. | i.v. | inactivated the pro- |
| | 100 ± 10 nm | Neutrophils | Injected once at 2 | inflammatory |
| | | | h after LPS | function of activated |
| | | | induction, mice | neutrophils |
| | | | lungs were | |
| | | | collected at 8 h | |
| | | | after induction | |
| Chu et al. | BSA NP ~130 | TPCA-1, a STAT3 | CD1 mice | Hijacked neutrophils |
| 2015 ^{[72]} | nm | and NF-κB | | for transporting anti- |
| | | inhibitor | | inflammatory drugs |
| | | N.A. | i.v. | across the air-blood |
| | | Neutrophils | Injected once at 4 | barrier (ABB) |
| | | | h after LPS | |
| | | | induction, mice | |
| | | | lungs were | |
| | | | collected at 20 h | |
| | | | after induction | |
| Spence et | Poly(lactic- | N.A. | C57BL/6 mice | Decreased the |
| al. 2015 ^{[73]} | co-glycolic) | di(α2→8) N- | i.p. | number of infiltrated |
| | acid (PLGA) | acetylneuraminic | | immune cells and |
| | NP | acid | | improved the |
| | | Macrophages | Injected once at 2 | survival rate |
| | | | h after LPS | |
| | | | induction, mice | |
| | | | were monitored | |
| | | | for 96 h | |
| Xu et al. | Cerium | N.A. | CBA/J mice | Reduced structural |
| 2016 ^{[74]} | Oxide NP | N.A. | i.p. | damage and collagen |
| | (CeO₂) | N.A. | Injected twice | deposition in the |
| | 38.11 ± 5.57 | | weekly for 4 | lungs, and |
| | nm | | weeks starting 2 | inflammatory |
| | | | h post-irradiation | response induced by |
| | | | | radiation |
| Li et al. | Liposome | Simvastatin | Balb/c mice | Attenuated TNF-α, |
| 2017 ^{[75]} | 354.7 ± 18.2 | Antibody against | i.v. | IL-6 and |
| | nm | intercellular | | inflammatory cells |
| | | adhesion molecule | | infiltration, superior |
| | | 1 (ICAM-1) | | efficacy than free |
| | | Endothelial cells | Injected once at 6 | drug. |
| | | | h after LPS | |
| | | | induction, mice | |
| | | | were sacrificed at | |
| | | | 24 or 48 h | |
| Li et al. | Liposome ~106 | Methylprednisolone | SD rats | Reduced levels of |
| 2017 ^{[76]} | nm | SP-A nanobodies | i.v. | TNF-α, IL-8, and |
| | | Lung surfactant | Continuous daily | TGF-β1 in |
| | | (lung lining fluid) | injections for 2 | bronchoalveolar |
| | | | weeks after | lavage fluid (BALF) |
| | | | disease induction | and the expression |
| | | | | of NK-κB in the |
| | | | | lung tissues |
| Gao et al. | Extracellular | Spleen tyrosine | CD1 mice | Reduced levels of |
| 2017 ^{[77]} | vesicles | kinase inhibitor | | TNF-α, IL-1β, and |
| | (EV) ~180 nm | Piceatannol | | IL-6 in blood |
| | | Neutrophil | i.v. | plasma and reduced |
| | | membrane | | neutrophil |
| | | Endothelial cells | Injected once at 2 | infiltration |
| | | | h after LPS | |
| | | | induction, mice | |
| | | | were sacrificed at | |
| | | | 12 h post | |
| | | | induction | |
| Zhu et al. | Se@SiO₂ NP ~55 | N.A. | SD rats | Reduced oxidative |
| 2017 ^{[78]} | nm | N.A. | i.p. | stress in lung tissues |
| | | N.A. | Injected daily for | and blood |
| | | | 3 days. First dose | |
| | | | was injected | |
| | | | together with the | |
| | | | induction drug | |
| Li et al. | Lipid NP | Dexamethasone | Balb/c mice | Attenuated |
| 2018 ^{[79]} | 249.9 ± 21.5 | Antibody against | i.v. | pulmonary |
| | nm | ICAM-1 | | inflammatory cell |
| | | Endothelial cells | Injected once at 6 | infiltration, and |
| | | | h after LPS | production of pro- |
| | | | induction, mice | inflammatory |
| | | | were sacrificed at | cytokines TNF-α |
| | | | 12 or 24 h post | and IL-6 |
| | | | induction | |
| Jiang et al. | Liposome | Simvastatin, | Balb/c mice | Reduced pulmonary |
| 2019 ^{[80]} | 351.7 ± 13.1 | protamine, and | | TNF-α and IL-6 |
| | nm | plasmid DNA | | levels, inflammatory |
| | | encoding the | | cell infiltration, and |
| | | angiopoietin-1 | | improved tissue |
| | | gene | | histology |
| | | Antibody against | i.v. | |
| | | ICAM-1 | | |
| | | Endothelial cells | Injected once at 6 | |
| | | | h after LPS | |
| | | | induction, mice | |
| | | | were sacrificed at | |
| | | | 12 h post | |
| | | | induction | |
| Liu et al. | BSA NP | Dexamethasone | Kunming mice | Reduced level of |
| 2019 ^{[81]} | 266.7 ± 2.7 nm | E-selectin-binding | i.v. | TNF-α in lung |
| | | peptide | | tissues and improved |
| | | Endothelial cells | Injected once at | histology |
| | | | 24 h after LPS | |
| | | | induction, mice | |
| | | | were sacrificed at | |
| | | | 48 h post | |
| | | | induction | |
| Molinaro et | Liposome ~94 | N.A. | Balb/c mice | Reduced lung |
| al. 2019 ^{[82]} | nm | Membrane proteins | i.v. | damage score |
| | | extracted from J774 | | |
| | | macrophages | | |
| | | Endothelial cells | Injected once at | |
| | | | 30 min after | |
| | | | induction, mice | |
| | | | were sacrificed at | |
| | | | 6 h post injection | |
| Shen et al. | Fe₃O₄ NP ~120 | N.A. | ICR mice | Reduced lung edema |
| 2019 ^{[83]} | nm | Macrophage | i.v. | and fever |
| | | membrane | | |
| | | Endothelial cells | NPs were | |
| | | | injected once | |
| | | | together with | |
| | | | LPS, mice were | |
| | | | sacrificed at 12 h | |
| | | | post induction | |
| Zhang et al. | Exosome ~100 | Syndecan-1 protein | C57BL/6 mice | Ameliorated lung |
| 2019 ^{[84]} | nm | Endothelial cell | i.v. | edema, number of |
| | | membrane | | cells and protein |
| | | Endothelial cells | Injected twice at | levels in BALF, and |
| | | | 2 and 12 h after | mitigated expression |
| | | | LPS induction, | of cytokines (IL-1β, |
| | | | mice were | TNF-α, and IL-6) |
| | | | sacrificed at 24 h | |
| | | | post induction | |
| Zhang et al. | Polymeric | STAT3 and NF-κB | CD1 mice | Targeted delivery to |
| 2019 ^{[85]} | micelle ~100 | inhibitor TPCA-1 | | acidic site of injury |
| | nm | Antibody against | i.v. | lung tissues, |
| | | ICAM-1 | | mitigated lung |
| | | Endothelial cells | Injected once at 4 | inflammation |
| | | | h after LPS | |
| | | | induction, mice | |
| | | | were sacrificed at | |
| | | | 20 h after | |
| | | | injection. | |
| Ma et al. | EV | STAT3 and NF-κB | Balb/c mice | Reduced levels of |
| 2020 ^{[86]} | 100-150 nm | inhibitor TPCA-1 | | cytokines (IL-1β, |
| | | Platelet membrane | i.v. | TNF-α, and IL-6), |
| | | Endothelial cells | Injected once at 4 | neutrophil |
| | | | h after LPS | infiltration, and ROS |
| | | | induction, mice | |
| | | | were sacrificed at | |
| | | | 20 h after | |
| | | | injection. | |
| Yang et al. | PLGA NP | Sparfloxacin and | ICR mice | Reduced levels of |
| 2020 ^{[87]} | 183.7 ± 9.4 nm | tacrolimus | | cytokine (TNF-α |
| | | γ3 peptide against | i.v. | and IL-6) and |
| | | ICAM-1 | | improved tissue |
| | | Endothelial cells | Injected once at 4 | histology |
| | | | h after disease | |
| | | | induction, mice | |
| | | | were sacrificed at | |
| | | | 20 h after | |
| | | | injection. | |
| Lee et al. | PLGA NP ~220 | DNase-1 | C57BL/6 mice | Reduced levels of |
| 2021 ^{[88]} | nm | Polydopamine- | i.v. | cytokines (IL-1β, |
| | | PEG | | TNF-α, IFN-γ, and |
| | | Neutrophils | Injected once at | IL-6), neutrophil |
| | | | 12 or 24 h after | infiltration, and |
| | | | disease induction, | edema |
| | | | mice sacrificed at | |
| | | | 72 h post | |
| | | | induction | |
| Niemiec et | CeO₂ NP ~190 | miR-146a | C57BL/6 mice | Decreased |
| al. 2022 ^{[89]} | nm | N.A. | i.t. | pulmonary leukocyte |
| | | Macrophages | NPs were | infiltration, |
| | | | injected once at 3 | inflammation and |
| | | | or 7 days post | oxidative stress, |
| | | | induction, tissues | pro-fibrotic gene |
| | | | were collected 14 | expression and |
| | | | days after | collagen deposition |
| | | | induction | in the lung |
| Arber | Liposome | Methylprednisolone | C57BL/6 mice | Reduced |
| Raviv et al. | 102.6 ± 0.3 nm | and N-acetyl | | inflammation and |
| 2022 ^{[90]} | | cysteine | | levels of TNF-α, IL- |
| | | Dipalmitoyl- | i.t. and i.v. | 6, and IL-1β |
| | | phosphatidylcholine | | cytokine secretion in |
| | | Both epithelial and | Injected once at 6 | BALF |
| | | endothelial cells | h post induction, | |
| | | | mice were | |
| | | | sacrificed at 26 h | |
| | | | post induction | |
| Wang et al. | Peptide | N.A. | C57BL/6 mice | Decreased |
| 2022 ^{[91]} | polymer NP | Peptide | i.t., i.p., or i.v. | pulmonary leukocyte |
| | 123.6 ± 3.9 nm | (CGLPFFD) | | infiltration and |
| | | conjugated with a | | levels of |
| | | fluorous tag bearing | | inflammatory |
| | | 13 fluorine atoms | | cytokines (TNF-α, |
| | | Macrophages | NPs were | IFN-γ, and IL-6) in |
| | | | injected 1 h prior | BALF |
| | | | to LPS induction, | |
| | | | mice were | |
| | | | sacrificed at 24 h | |
| | | | post induction | |
| Zhang et al. | nanoparticle- | Ciprofloxacin | CD1 mice | Significant anti- |
| 2022 ^{[92]} | modified | Neutrophil | i.t. | bacterial efficacy |
| | algae ~6 μm | membrane | | without in vivo |
| | | Endothelial cells | Injected once at | toxicity |
| | | | 0.5 h post | |
| | | | induction, mice | |
| | | | were monitored | |
| | | | for 2 weeks | |
| Muhammad | Polyurethane | Dexamethasone | C57BL/6 mice | Reduced levels of |
| et al. | NP | ROS-responsive | Inhalation | BALF and serum |
| 2022 ^{[93]} | 251 ± 3 nm | thioketal bonds | | cytokines (TNF-α, |
| | | Macrophages and | Daily inhalation | IFN-γ, and IL-6), |
| | | neutrophils | for 3 days | ROS, and lung |
| | | | starting from 4 h | damage score |
| | | | post induction | |
| Meng et al. | EV ~90 nm | Dexamethasone | K18-hACE2 | Reduced |
| 2023 ^{[94]} | | | mice, Balb/c | inflammation |
| | | | mice, and rhesus | (decreased cytokines |
| | | | macaques | and cell infiltration) |
| | | Neutrophil | Inhalation | without significant |
| | | membrane | | osteoporosis in |
| | | Macrophages | K18-hACE2 | bacterial or viral |
| | | | mice: two daily | infection |
| | | | inhalations | |
| | | | starting froms 2 | |
| | | | days post viral | |
| | | | infection; | |
| | | | Balb/c mice: | |
| | | | inhalation once at | |
| | | | 4 h post LPS | |
| | | | induction; | |
| | | | rhesus macaques: | |
| | | | 6 daily | |
| | | | inhalations | |
| | | | starting from 1 | |
| | | | day post viral | |
| | | | infection | |
| Fei et al. | Liposome | miR-146a | C57BL/6 mice | Reduced levels of |
| 2023 ^{[95]} | 218.7 ± 13.2 | Mannose | i.t. | CXCL1 and IL-6 in |
| | nm | Macrophages | Injected once | BALF |
| | | | right after | |
| | | | induction | |
| Zhao et al. | Liposome | siRNA targeting | C57BL/6 mice | Reduced BALF |
| 2024 ^{[96]} | 100-240 nm | TGF-β-activated | | cytokine levels |
| | | kinase 1 | | (TNF-α, IL-1β, and |
| | | Macrophage- | i.n. | IL-6), BALF total |
| | | specific antibody | | protein, and lung |
| | | F4/80 | | damage area |
| | | Macrophages | Two injections at | |
| | | | Day 15 and Day | |
| | | | 20 post viral | |
| | | | infection | |
| Huang et | Metal- | fibroblast growth | C57BL/6 mice | Improved lung tissue |
| al. 2024 ^{[97]} | organic | factor 21 | | morphology and, |
| | framework | Apoptotic MLE-12 | i.p. | decrease cytokines |
| | 137.6 ± 6.2 nm | cell membrane | | level (CXCL15, |
| | | General lung tissues | Injected once at 4 | TNF-α, IL-1β, and |
| | | | h post induction, | IL-6) in both tissue |
| | | | tissues were | and BALF |
| | | | collected at 24 h | |
| | | | after injection | |
| Wang et al. | PLGA ~84 nm | Curcumin and | C57BL/6 mice | ROS scavenging in |
| 2024 ^{[98]} | | bardoxolone | | the lungs and |
| | | Bardoxolone- | i.v. | activated Nrf2 |
| | | loaded MLE-12 | | pathway |
| | | cell membrane | | |
| | | General lung tissues | Injected 2 h prior | |
| | | | to induction, | |
| | | | tissues were | |
| | | | collected 4 h post | |
| | | | induction | |
| Peng et al. | Cerium- | N.A. | ICR mice | Reduced ROS |
| 2024 ^{[99]} | based tannic | ROS-responsive | i.n. | levels, inflammatory |
| | acid | peptides | | cytokine production, |
| | nanozymes | Inflammatory sites | Viral infection | and promoted |
| | coated with | | model: two daily | macrophage |
| | ROS- | | nasal instillations | polarization to the |
| | responsive | | from 48 h post | prohealing (M2) |
| | peptides | | induction; | phenotype |
| | 100-275 nm | | Viral-bacterial | |
| | | | co-infection | |
| | | | model: three | |
| | | | daily nasal | |
| | | | instillations from | |
| | | | 24 h post | |
| | | | induction | |
| Solé-Porta | PLGA nano- | Human serum | SD rats | Homogeneous |
| et al. | capsules ~210 | albumin | | distribution of |
| 2024 ^{[15]} | nm | N.A. | Inhalation | nanocapsules to type |
| | | Type II alveolar | Instillation with | II alveolar epithelial |
| | | epithelial cells | HCl and LPS | cells in the lungs, |
| | | | with 2 h | reduced |
| | | | intervals. | macrophage- |
| | | | Inhalation started | mediated lung |
| | | | 7 h post- | clearance |
| | | | induction; | |
| | | | sacrifice 16 h | |
| | | | post-inhalation | |

Abbreviations: i.v.: intravenous injection; i.p.: intraperitoneal injection; i.n.: intranasal injection; BALF: bronchoalveolar lavage fluid; TNF: tumor necrosis factor; IL: interleukin; IFN: interferon; PEG: polyethylene glycol; N.A.: not applicable; STAT3: signal transducer and activator of transcription 3; NF-κB: nuclear factor kappa-B; SP-A: surfactant protein A; TGF: transforming growth factor; CXCL: chemokine (C-X-C motif) ligand; MLE: murine lung epithelial cells; ROS: reactive oxygen species.

TABLE 2
Summary of Gold NP-containing Therapies for ARDS

| | Targeting ligand | Delivery | Animal | |
|---|---|---|---|---|
| Reference | or coating | route | model | Main therapeutic outcomes |
| Casagrande | Curcumin | i.n. | LPS-induced | Decreased levels of pro- |
| et al. 2023 ^{[49]} | ~20 nm | | Wistar rats | inflammatory cytokines |
| | | | | (TNF-α and IFN-γ) in BALF, |
| | | | | increased levels of anti- |
| | | | | inflammatory cytokines (IL- |
| | | | | 10, IL4, and TGF-β) levels, |
| | | | | relieved oxidative stress in |
| | | | | the lungs |
| dos Santos | N.A. | i.p. | LPS-induced | Reduced leukocytes count, |
| Haupenthal | ~20 nm | | Wistar rats | cytokine level, and alveolar |
| et al. 2020 ^{[100]} | | | | wall thickness |
| Wang et al. | P12 peptide | i.t. | LPS-induced | Increased M2 macrophages |
| 2020 ^{[101]} | 18.8 ± 0.1 nm | | C57BL/6 | and reduced M1 macrophages |
| | | | mice | in the BALF and lung tissues. |
| | | | | Reduced proinflammatory |
| | | | | cytokines |
| Gao et al. | P12 peptide (with | i.t. | LPS-induced | Prolonged survival, reduced |
| 2019 ^{[102]} | different gold | | C57BL/6 | levels of cytokines in BALF, |
| | core sizes) | | mice | and repair of alveolar damage |
| Gao et al. | P12 peptide and | i.t. | LPS-induced | Enhanced anti-inflammation |
| 2019 ^{[103]} | surface | | C57BL/6 | through TLR inhibition and |
| | modification with | | mice | autophagy induction |
| | cigarette smoke | | | |
| | extract | | | |
| | 23.9 ± 0.3 nm | | | |
| Xiong et al. | P12 peptide | i.t. | LPS-induced | Reduced cell infiltration in |
| 2018 ^{[29]} | 17.5 ± 0.6 nm | | C57BL/6 | the lungs and improved tissue |
| | | | mice | morphology as well as |
| | | | | reduced liver accumulation |

Abbreviations: i.n.: intranasal injection; i.p.: intraperitoneal injection; i.t.: intratracheal injection; LPS: lipopolysaccharide; BALF: bronchoalveolar lavage fluid; N.A.: not applicable; TNF: tumor necrosis factor; IL: interleukin; IFN: interferon; P12: a 14-amino-acid peptide (sequence: PSHISKYILRWRPK (SEQ ID NO: 1)); TLR: toll-like receptor.

TABLE 3
Inhalable Spike/RBD-conjugated NPs (Note: These are the only available
Spike-RBD-conjugated NPs in the literature. In all examples, the NP
served as a vaccine rather than treatment of lung inflammation.)

| Reference | NP and size | Animal | Main outcomes |
|---|---|---|---|
| Wang et | Exosome | CD1 mice | Protection from infection by SARS-CoV-2 |
| al. 2022 ^{[104]} | ~100 nm | and Syrian | pseudovirus with RBD-specific IgG |
| | | hamsters | antibodies, mucosal IgA responses and T cells |
| | | | with a Th1-like cytokine expression profile in |
| | | | lungs. Attenuated pneumonia and |
| | | | inflammatory infiltrates in SARS-CoV-2 |
| | | | infected hamsters |
| Zhuo et al. | Chitosan | Balb/c mice | Induction of Spike-specific IgG and IgA in |
| 2022 ^{[105]} | polymers | | BALF and T-cell response in the spleen upon |
| | 210.3 ± 2.1 nm | | inhalation, comparable antibody immune |
| | | | response to intramuscular injection |
| Elder et al. | ISR52, | AC70 | Induction of Spike-specific IgA/IgG in BALF |
| 2023 ^{[106]} | Unknown | hACE2 | and IgG in serum in hACE2 transgenic mice, |
| | structure and | transgenic | induction of Spike-specific T-cell responses |
| | size | mice | over 6 months in wild-type mice |
| Ye et al. | Cholera toxin B | K18 hACE2 | Alveoli delivery, sustained antigen release, |
| 2023 ^{[107]} | subunit (CTB)- | transgenic | and antigen-presenting cell uptake, strong |
| | based NP in a | mice, Syrian | production of IgG and IgA in BALF and |
| | microcapsule | hamsters, and | serum, local T cell response, protection |
| | NP size: ~24 nm | cynomolgus | against SARS-CoV-2 in mice, hamsters and |
| | Microcapsule | monkeys | nonhuman primates |
| | size: 2-4 μm | | |
| Wang et | Liposome | C57BL/6 | Activation of alveolar macrophage, T cells, |
| al. 2024 ^{[108]} | ~154 nm | mice | and B cells, increased levels of RBD-specific |
| | | | serum IgG and BALF secretory IgA |

TABLE 4
cDNA Sequence of His-tagged-Spike Receptor Binding Domain (RBD) with His
Tag (underlined bolded text).

| Human codon-optimized RBD^{[109]} | Bacterial codon-optimized RBD |
|---|---|
| (5′→3′) (SEQ ID NO: 2) | (5′→3′) (SEQ ID NO: 3) |
| ATGAAGACCATCATCGCCCTGAGCTAC | ATGAAAACAATTATAGCTCTATCATAT |
| ATCTTCTGCCTGGTGTTCGCCACTAATC | ATATTCTGCCTGGTGTTTGCGACCAA |
| TTTGTCCGTTCGGTGAGGTTTTTAACG | CTTATGCCCGTTTGGTGAGGTGTTCA |
| CGACAAGGTTCGCTAGTGTATATGCTT | ACGCAACCCGTTTTGCCTCTGTTTAC |
| GGAACCGAAAGAGAATCTCCAATTGC | GCGTGGAATCGTAAGCGCATCAGCA |
| GTAGCTGATTACTCCGTTCTCTATAACA | ATTGCGTTGCGGACTACAGCGTCTTG |
| GTGCGTCCTTTTCAACCTTTAAGTGTTA | TACAACAGCGCATCCTTTAGCACCTT |
| CGGCGTTTCTCCAACGAAGCTGAATGA | TAAATGCTACGGCGTAAGCCCGACTA |
| TCTCTGTTTTACGAACGTGTATGCTGAC | AGCTGAATGATCTGTGCTTTACCAAC |
| TCTTTCGTTATACGGGGGGACGAAGTG | GTCTATGCGGACTCGTTTGTTATCCG |
| AGACAGATAGCACCAGGTCAGACTGG | TGGTGATGAAGTTCGTCAGATCGCTC |
| GAAGATAGCGGATTACAACTATAAGTT | CGGGTCAAACCGGCAAAATCGCGGA |
| GCCCGATGATTTTACGGGGTGCGTAAT | CTATAACTACAAACTGCCGGATGACT |
| CGCATGGAACTCAAACAACCTCGACTC | TCACAGGTTGTGTTATTGCGTGGAAT |
| CAAAGTAGGTGGTAATTATAATTACTTG | TCCAACAACCTTGATAGCAAGGTGG |
| TATCGCCTGTTTCGAAAGAGCAATTTG | GCGGCAACTACAATTATCTGTACAGA |
| AAGCCTTTTGAGCGGGATATTTCAACC | TTGTTCCGCAAGTCTAACCTGAAACC |
| GAAATTTACCAAGCAGGCAGTACGCCA | GTTCGAGCGTGACATTTCCACGGAA |
| TGTAACGGAGTAGAGGGATTTAATTGC | ATTTATCAAGCTGGCTCTACCCCGTG |
| TACTTTCCTCTTCAATCTTATGGCTTTC | TAATGGCGTGGAAGGCTTCAACTGCT |
| AACCAACAAACGGAGTGGGGTATCAA | ACTTCCCACTGCAGAGCTATGGTTTC |
| CCTTATAGAGTGGTAGTATTGTCCTTTG | CAACCGACGAACGGTGTTGGCTATC |
| AGCTCCTCCACGCCCCGGCTACAGTTT | AGCCGTATCGCGTGGTGGTGCTGAG |
| GTGGGCCCAAAAAGGGATCCGGTCAC | CTTCGAGTTGCTCCATGCCCCTGCTA |
| CATCACCACCACCATCATCATTGA | CCGTTTGTGGTCCGAAAAAGGGTTC |
| | AGGTCATCACCACCATCACCACCA |
| | CCATTAA |

We chose the Spike RBD sequence based on plasmid pcDNA3-SARS-COV-2-S-RBD-8his (Addgene #145145) from Procko's lab (left column); this plasmid was originally used for expressing Spike RBD in eukaryotic HEK cells^{[109]}. For expression in prokaryotic E. Coli cells (out of convenience and cost reduction as we require large amounts of proteins for our efficacy studies), we sought codon optimization and plasmid construction service from GenScript to build a prokaryotic version of this plasmid pET11a-RBD-8his (right column).

TABLE 5
Physicochemical Characterization of Other Gold NPs

| | Hydrodynamic | | | | |
|---|---|---|---|---|---|
| | diameter (nm) | Zeta | PEG loading (strands/NP) | | |
| | (Polydispersity | potential | PEG_{20K}- | PEG₂₀ₖ- | PEG₁₀ₖ- |
| NP | index) | (mV) | methoxy | RBD | Cy5.5 |
| Au core | 23.9 ± 0.96 | −15.27 ± 1.30 | / | / | / |
| | (0.046) | | | | |
| Au@PEG- | 95.0 ± 3.41 | −18.46 ± 2.66 | 626.33 ± 13.76 | / | / |
| methoxy | (0.034) | | | | |
| Au@PEG- | 94.2 ± 1.87 | −14.25 ± 2.87 | 335.98 ± 10.57 | 312.38 ± 9.28 | / |
| RBD | (0.033) | | | | |
| Au@PEG- | 93.6 ± 1.95 | −25.12 ± 1.28 | / | 639.59 ± 11.61 | / |
| RBD (100% | (0.057) | | | | |
| RBD loaded) | | | | | |
| Au@PEG- | 93.2 ± 3.57 | −16.99 ± 4.23 | 619.27 ± 20.45 | / | 31.72 ± 4.19 |
| methoxy/Cy5.5 | (0.044) | | | | |
| (96% | | | | | |
| methoxy/4% | | | | | |
| Cy5.5) | | | | | |
| Au@PEG- | 92.5 ± 1.33 | −15.92 ± 1.23 | 311.76 ± 11.82 | 301.65 ± 7.87 | 34.63 ± 5.23 |
| RBD/Cy5.5 | (0.052) | | | | |
| (50% | | | | | |
| RBD/46% | | | | | |
| methoxy/4% | | | | | |
| RBD) | | | | | |

| | Aerosol concentration (×10⁶ | Airbone NP diameter (nm, |
|---|---|---|
| | NPs/cm³, measured by a scanning | measured by a scanning mobility |
| | mobility particle sizer) | particle sizer) |
| Au@PEG- | 9.41 ± 0.33 | 90.11 ± 2.36 |
| methoxy | | |
| Au@PEG- | 9.12 ± 0.16 | 98.57 ± 1.69 |
| RBD | | |

TABLE 6
Physicochemical characterization of Gold NPs upon
Incubation in Fresh BALF Supernatant (from healthy
hamsters) at 37° C. before DLS measurements.

| | Hydro- | | |
|---|---|---|---|
| | dynamic | Zeta | Poly- |
| | diameter | potential | dispersity |
| NP | (nm) | (mV) | index |
| Au@PEG-methoxy/ | 119.2 ± 3.83 | −18.79 ± 4.66 | 0.102 |
| Cy5.5 in BALF (30 min) | | | |
| Au@PEG-methoxy/ | 118.5 ± 4.55 | −17.55 ± 2.87 | 0.125 |
| Cy5.5 in BALF (24 h) | | | |
| Au@PEG-RBD/Cy5.5 | 120.0 ± 3.11 | −15.45 ± 2.15 | 0.133 |
| (50% RBD loaded) in | | | |
| BALF (30 min) | | | |
| Au@PEG-RBD/Cy5.5 (50% | 124.7 ± 2.36 | −16.78 ± 3.44 | 0.099 |
| RBD loaded) in BALF (24 h) | | | |
| Au@PEG-methoxy in BALF | 116.8 ± 1.58 | −19.26 ± 2.77 | 0.105 |
| (30 min) | | | |
| Au@PEG-methoxy in BALF | 118.2 ± 2.45 | −15.63 ± 4.88 | 0.137 |
| (24 h) | | | |
| Au@PEG-RBD (50% RBD | 119.7 ± 3.87 | −16.58 ± 3.85 | 0.114 |
| loaded) in BALF (30 min) | | | |
| Au@PEG-RBD (50% RBD | 116.9 ± 2.98 | −18.26 ± 2.11 | 0.108 |
| loaded) in BALF (24 h) | | | |

TABLE 7
Rating scale of lung damage score

| Score | Criteria |
|---|---|
| 1 | No visible lesions |
| 2 | <25% tissue affected |
| 3 | 25-50% tissue affected |
| 4 | 50-75% tissue affected |
| 5 | >75% tissue affected |

TABLE 8
Top 50 Inhibited Kinases by Au@PEG-methoxy
NP from Kinome Profiling of 281 kinases

| Kinase | % Inhibition | ATP concentration |
|---|---|---|
| MAPK 14 (p38α) | 92 | 100 μM |
| RSK4 | 83 | Km apparent (30 μM) |
| FGR | 76 | Km apparent (10 μM) |
| GRK4 | 74 | Km apparent (12 μM) |
| PLK3 | 70 | Km apparent (47.8 μM) |
| PLK2 | 69 | Km apparent (29.6 μM) |
| PDK1 Direct | 67 | 100 μM |
| JAK2 | 65 | Km apparent (31 μM) |
| MAP2K6 (MKK6) | 62 | 100 μM |
| FER | 60 | Km apparent (18.5 μM) |
| AMPK A2/B1/G1 | 56 | Km apparent (148 μM) |
| RPS6KA6 (RSK4) | 54 | Km apparent (30 μM) |
| JAK3 | 49 | Km apparent (14 μM) |
| MAP4K5 (KHS1) | 49 | Km apparent (55 μM) |
| RPS6KA4 (MSK2) | 48 | Km apparent (14.1 μM) |
| IKBKB (IKK beta) | 46 | Km apparent (5 μM) |
| SGK (SGK1) | 46 | Km apparent (36 μM) |
| GRK6 | 45 | Km apparent (11.7 μM) |
| YES1 | 45 | Km apparent (20.3 μM) |
| NEK1 | 44 | Km apparent (118.7 μM) |
| ADRBK2 (GRK3) | 43 | Km apparent (11.7 μM) |
| FYN | 42 | Km apparent (85 μM) |
| SGK2 | 41 | Km apparent (50 μM) |
| STK22B (TSSK2) | 39 | Km apparent (3 μM) |
| BRSK1 (SAD1) | 39 | Km apparent (32 μM) |
| TEK (TIE2) Y897S | 39 | Km apparent (207 μM) |
| CLK2 | 38 | Km apparent (30 μM) |
| TYRO3 (RSE) | 37 | Km apparent (27.7 μM) |
| AKT2 (PKB beta) | 37 | Km apparent (200 μM) |
| DYRK3 | 36 | Km apparent (4.7 μM) |
| BTK | 36 | Km apparent (36 μM) |
| MET (cMet) | 35 | Km apparent (64 μM) |
| ABL2 (Arg) | 33 | Km apparent (30 μM) |
| AURKA (Aurora A) | 33 | Km apparent (10 μM) |
| ABL1 T315I | 32 | Km apparent (2.8 μM) |
| EPHA4 | 32 | Km apparent (106 μM) |
| JAK2 JH1 JH2 | 32 | Km apparent (46 μM) |
| PRKCA (PKC alpha) | 32 | Km apparent (35 μM) |
| STK23 (MSSK1) | 31 | Km apparent (69 μM) |
| MUSK | 31 | Km apparent (49.5 μM) |
| ABL1 E255K | 30 | Km apparent (4.2 μM) |
| PIM2 | 30 | Km apparent (3 μM) |
| RET V804E | 30 | Km apparent (50 μM) |
| RPS6KB1 (p70S6K) | 30 | Km apparent (17 μM) |
| PLK1 | 29 | Km apparent (12.8 μM) |
| AURKC (Aurora C) | 29 | Km apparent (26 μM) |
| AMPK A1/B1/G1 | 28 | Km apparent (43 μM) |
| ROS1 | 27 | Km apparent (61.4 μM) |
| EPHB4 | 27 | Km apparent (115 μM) |
| AMPK (A2/B1/G2) | 27 | Km apparent (11 μM) |

TABLE 9
Proteomics Analysis of Differential Expressed Proteins

| | Expression level | |
|---|---|---|
| | (Au@PEG-methoxy NP/ | Adj. |
| Proteins | untreated) | p value |
| Ltc4s | 0.114/0.020 | 0.047 |
| Thbs1 | 0.383/0.084 | 0.031 |
| Histone H2A | 2.584/0.711 | 0.019 |
| Cryab | 0.307/0.756 | 0.050 |
| Strap | 0.038/0.146 | 0.047 |
| Plcb3 | 0.020/0.079 | 0.049 |
| Sod2 | 0.112/0.455 | 0.044 |
| Dpysl3 | 0.036/0.151 | 0.049 |
| Camk2b | 0.053/0.222 | 0.048 |
| Tardbp | 0.050/0.219 | 0.035 |
| Srsf1 | 0.030/0.131 | 0.041 |
| Itih4 | 0.038/0.172 | 0.030 |
| Rpl32 | 0.056/0.254 | 0.032 |
| F2 | 0.051/0.251 | 0.040 |
| Cttn | 0.021/0.108 | 0.036 |
| Hpgd | 0.035/0.196 | 0.049 |
| Apoe | 0/0.183 | 0.004 |
| Elavl1 | 0/0.170 | 0.002 |
| Rad23b | 0/0.125 | 0.001 |
| Ephx2 | 0/0.116 | 0.001 |

TABLE 11
Past clinical reports of corticosteroids for treating ARDS.

| Author | ARDS | Number of | | Outcomes related | |
|---|---|---|---|---|---|
| (year) | subtypes | patients | Dosage regimen | to this work | Other outcomes |
| Annane | Septic | 177 | 50 mg/6 h, 7 d | IL-6 plasma levels | A 7-day treatment with low doses of |
| et al. | shock- | (hydrocortisone, n = | | were significantly | corticosteroids was associated with a better |
| (2006) | associated | 85; placebo, n = | | lower in the steroid | survival rate (hazard ratio 0.57, 95% CI |
| ^{[53]} | early ARDS | 92) | | group than in the | 0.36 to 0.89, p = 0.013; relative risk |
| | | | | placebo group | 0.71, 95% CI 0.54 to 0.94, p = 0.011) |
| | | | | | and less ventilation-free days in |
| | | | | | septic shock-associated early ARDS |
| | | | | | nonresponders. |
| Steinberg | Persistent | 180 | A loading dose of | / | The results do not support the routine use of |
| et al. | ARDS | (methylprednisolone, | 2 mg/kg body | | methylprednisolone for persistent ARDS. In |
| (2006) | | n = 89; placebo, | weight. Treatment | | addition, starting methylprednisolone therapy |
| ^{[110]} | | n = 91) | was continued at | | more than two weeks after the onset of |
| | | | 0.5 mg/kg body | | ARDS may increase the risk of death. |
| | | | weight every 6 | | |
| | | | hours for 14 days, | | |
| | | | 0.5 mg/kg body | | |
| | | | weight every 12 | | |
| | | | hours for 7 days, | | |
| | | | and then tapering | | |
| | | | of the dose. | | |
| Meduri | Early | 91 | A loading dose of | The | Methylprednisolone reduce the duration of |
| et al. | Severe | (methylprednisolone, | 1 mg/kg was | methylprednisolone- | mechanical ventilation and ICU length of stay. |
| (2007) | ARDS | n = 63; placebo, | followed by 1 | treated group had | |
| ^{[55]} | | n = 28) | mg/kg/d from day | a significant | |
| | | | 1 to day 14, 0.5 | reduction in C- | |
| | | | mg/kg/d from day | reactive protein | |
| | | | 15 to day 21, 0.25 | (p < 0.0001), Lung | |
| | | | mg/kg/d from day | Injury Score (p = | |
| | | | 22 to day 25, and | 0.002) and | |
| | | | 0.125 mg/kg/d | significantly | |
| | | | from day 26 to day | improved lung | |
| | | | 28. | function [higher | |
| | | | | PaO₂/FiO₂ ratio (p = | |
| | | | | 0.006)] from day | |
| | | | | 3, | |
| Liu | Early ARDS | 26 | 100 mg/8 h, 7 d | / | Stimulate dose of hydrocortisone could reduce |
| et al. | combined | (hydrocortisone, n = | | | shock incidence and lower 28-day mortality in |
| (2012) | with critical | 12; placebo, n = | | | early ARDS patients combined with CIRCI. |
| ^{[111]} | illness- | 14) | | | |
| | related | | | | |
| | corticosteroid | | | | |
| | insufficiency | | | | |
| | (CIRCI) | | | | |
| Tongyoo | Early sepsis- | 197 | 50 mg/6 h, 7 d | The hydrocortisone | Early administration of low-dose hydrocortisone |
| et al. | associated | (hydrocortisone, n = | | group had a | was safe but did not yield a significant survival |
| (2016) | ARDS | 98; placebo, n = | | significantly lower | benefit. |
| ^{[56]} | | 99) | | Lung Injury Score | |
| | | | | (p = 0.003) and | |
| | | | | associated with | |
| | | | | improved lung | |
| | | | | function [higher | |
| | | | | PaO₂/FiO₂ ratio (p = | |
| | | | | 0.03)] from day | |
| | | | | 3. | |
| Dequin | COVID-19 | 149 | A loading dose of | / | Low-dose hydrocortisone, compared with placebo, |
| et al. | and acute | (hydrocortisone, n = | 200 mg/d. | | did not significantly reduce treatment failure at |
| (2020) | respiratory | 76; placebo, n = | Treatment was | | day 21. (absolute difference −8.6 percentage |
| ^{[112]} | failure | 73) | continued at 200 | | points, 95% CI, −24.9 to 7.7, p = 0.29) |
| | | | mg/d until day 7 | | |
| | | | and then decreased | | |
| | | | to 100 mg/d for 4 | | |
| | | | days and 50 mg/d | | |
| | | | for 3 days, for a | | |
| | | | total of 14 days. | | |
| Edalatifard | COVID-19- | 68 | 250 mg/d, 3d | The | Methylprednisolone group had a significantly |
| et al. | associated | (methylprednisolone, | | methylprednisolone- | increased survival time compared to placebo group |
| (2020) | ARDS | n = 34; placebo, | | treated group had | (hazard ratio 0.293, 95% CI 0.154 to 0.556, p < |
| ^{[54]} | | n = 34) | | a significant | 0.001). |
| | | | | reduction in C- | |
| | | | | reactive protein | |
| | | | | (p < 0.001) and | |
| | | | | serum IL-6 | |
| | | | | (p < 0.001) | |
| | | | | from day 3, | |
| Tomazini | COVID-19- | 299 | 20 mg/d for 5 days, | / | Dexamethasone significantly increase the number |
| et al. | associated | (dexamethasone, n = | followed by 10 | | of ventilator-free days over 28 days (absolute |
| (2020) | ARDS | 151; placebo, n = | mg/d for another 5 | | difference 2.26 days, 95% CI, 0.2 to 4.38, p = |
| ^{[113]} | | 148) | days | | 0.04). |
| Villar | Moderate- | 277 | 20 mg/d for 5 days, | The | Dexamethasone significantly increase the number |
| et al. | to-severe | (dexamethasone, n = | followed by 10 | dexamethasone- | of ventilator-free days over 28 days (absolute |
| (2020) | ARDS | 139; placebo, n = | mg/d for another 5 | treated group had | difference 4.8 days, 95% CI, 2.57 to 7.03, p < |
| ^{[114]} | | 138) | days | an improved lung | 0.0001). |
| | | | | function in terms | |
| | | | | of PaO₂/FiO₂ ratio | |
| | | | | from day 6. | |
| Kwon | Influenza A | 18106 | Hydrocortisone, | / | Dexamethasone yield best efficacy (odds ratio) |
| et al. | (H1N1)- | (H1N1 ARDS, n = | methylprednisolone, | | among three steroids in terms of long-term |
| (2022) | associated | 3461; non-viral | or dexamethasone | | mortality. |
| ^{[115]} | ARDS; | ARDS, n = 6862; | (unspecified dose) | | |
| | non-viral | COVID-19 ARDS, | | | |
| | ARDS; | n = 7783) | | | |
| | COVID-19- | | | | |
| | associated | | | | |
| | ARDS | | | | |
| Dequin | Severe | 795 | 200 mg daily for | / | Patients received hydrocortisone had a lower risk |
| et al. | community- | (hydrocortisone, n = | either 4 or 7 days | | of death by day 28 than those who received |
| (2023) | acquired | 400; placebo, n = | as determined by | | placebo. (absolute difference −5.6 percentage |
| ^{[116]} | pneumonia | 395) | clinical | | points, 95% CI, −9.6 to −1.7, p = 0.006) |
| | | | improvement, | | |
| | | | followed by | | |
| | | | tapering for a total | | |
| | | | of 8 or 14 days | | |

### DETAILED DISCLOSURE OF THE INVENTION

Embodiments of the subject invention are directed to a method for preparing inhalable targeted gold nanoparticles for accelerated lung delivery and treating lung inflammation.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

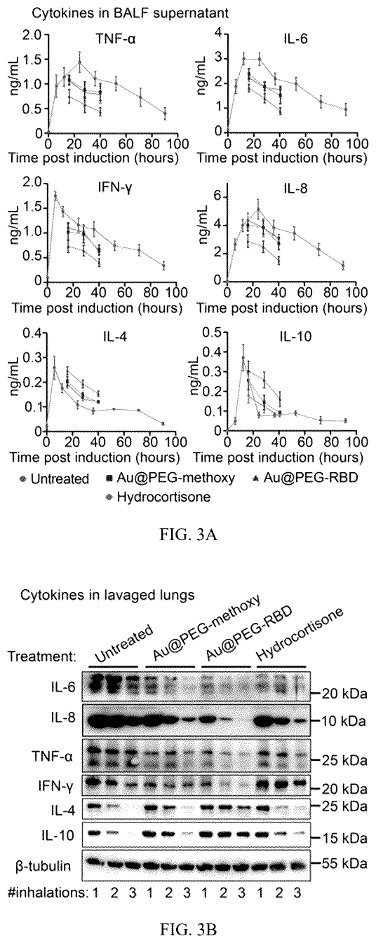

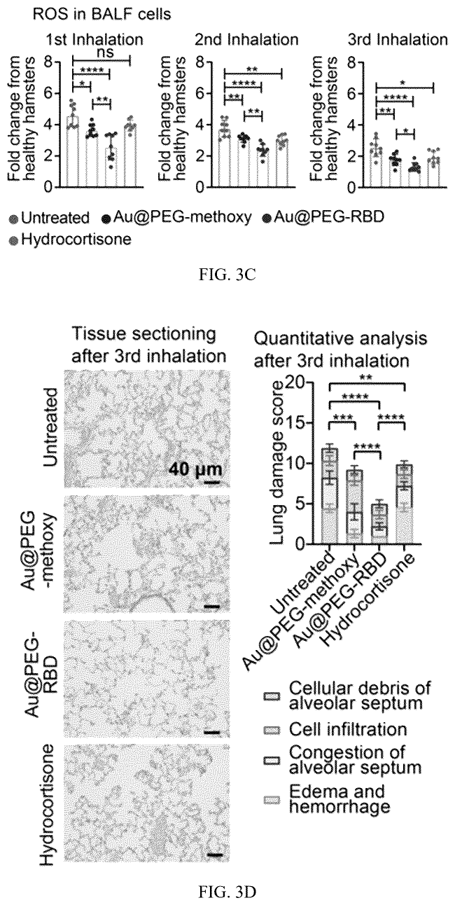

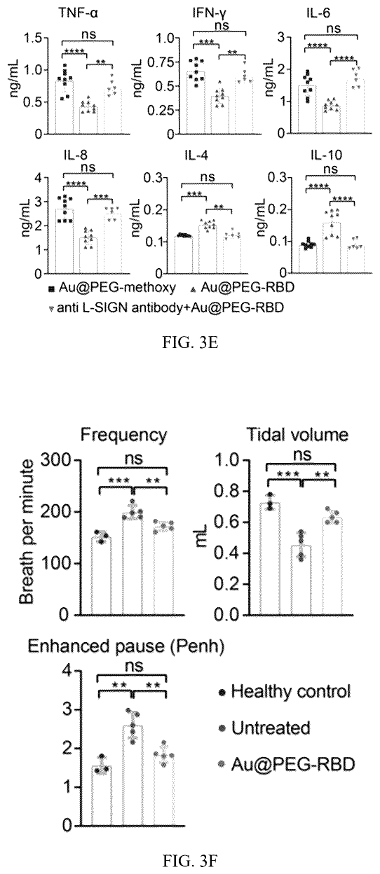

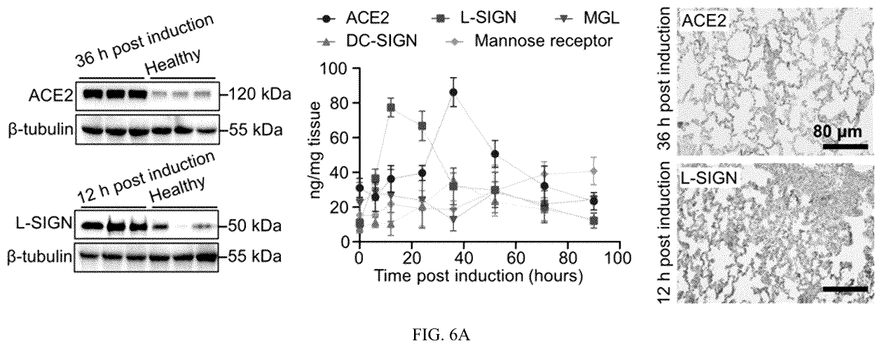

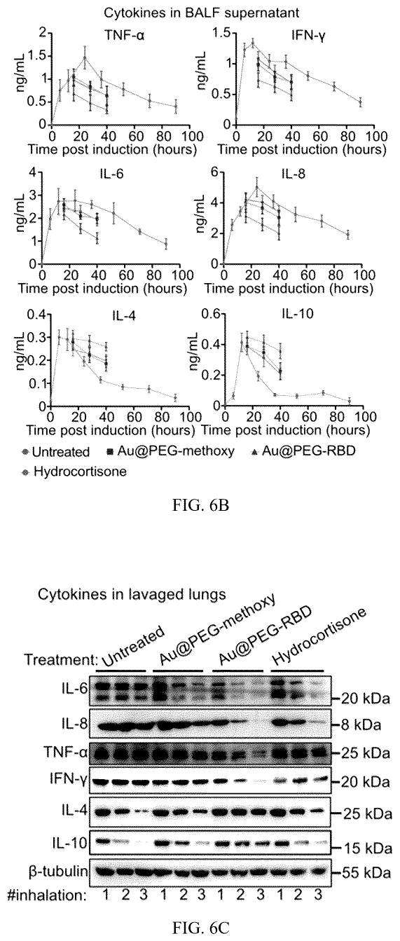

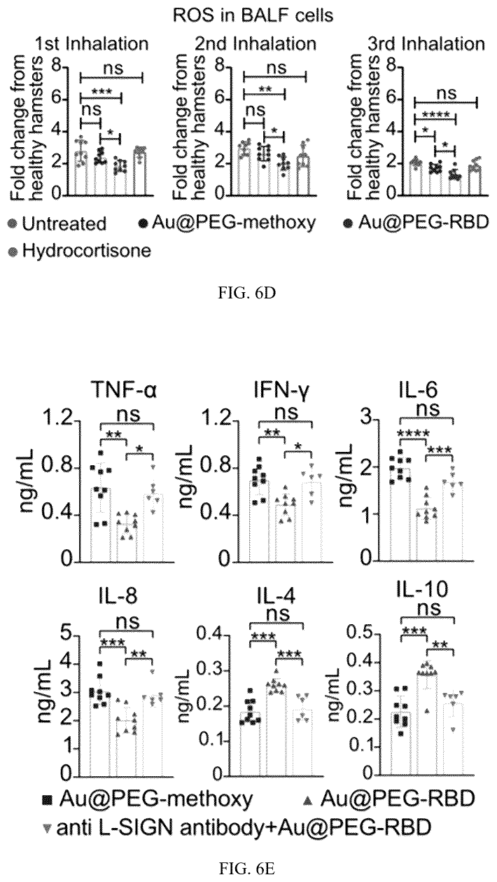

A rapid, noninvasive, and disease-driven nanomedicine for ARDS is provided, being based on a gold NP conjugated with multiple recombinant Spike receptor binding domain (RBD) subunit proteins (which originate from the SARS-COV-2 virus) for inhalation delivery to epithelial cells (which occupy about 90% of lung cells). This bioinspiration approach stems from (i) rapid infection of lung epithelium by inhaled SARS-COV-2 viruses via the Spike receptor, (ii) clinically validated safety of RBD-containing NPs as COVID-19 vaccines (see Table 3), and (iii) discovery of the transient, sequential activation of two Spike receptors on the lung epithelium induced by ARDS at different timepoints post-ARDS onset, 12 hours for liver/lymph node-specific intercellular adhesion molecule-3-grabbing integrin (L-SIGN) followed by 36 hours for angiotensin-converting enzyme 2 (ACE2). The therapeutic premise is to enhance the inhalation delivery of self-therapeutic gold NP to epithelial cells in the ARDS lung via recombinant Spike RBD proteins to engage activated L-SIGN and ACE2 receptors; both receptors mediate alveolar targeting of the nanomedicine additively. The two ARDS models selected are Syrian hamsters (which have similar Spike receptors to humans^{[16]}) with lipopolysaccharides (LPS)-induction (which corresponds to bacterial infection; see FIG. 3) and hydrochloric acid (HCl)-induction (which corresponds to aspiration; see FIG. 6). Both non-viral ARDS are applicable to the broader patient population. Moreover, no viral blockade mechanism or COVID-19-associated ARDS animal model is involved despite the conjugation of Spike RBD proteins on the gold NP. For the pharmacological mechanism, the gold core naturally inhibits p38a mitogen-activated protein kinase (MAPK) phosphorylation and polo-like kinase 3 (PLK3), both therapeutic targets of ARDS, without chemical drugs, biologics, or external physical forces. The shell of polyethylene glycol (PEG) strands endows the NP with stability upon aerosolization and in bronchoalveolar lavage fluid (BALF), conjugates RBD to the gold core. The overall NP size is about 94 nm, large enough to inhibit ABB penetration to systemic circulation^{[17]} yet small enough for homogeneous alveolar distribution^{[18]}. Upon intermittent inhalations for 6 hour, the NP deposits in ARDS lungs more abundantly than healthy lungs and treats ARDS more effectively than standard steroid therapy in both hamster models without long-term retention in major organs or toxicity one year post inhalation.

According to the embodiments of the subject invention, spike receptor binding domain (RBD)-conjugated gold NP for inhalation delivery to lung epithelial cells and treating ARDS in hamsters are provided. The rapid infection of human lung cells by inhaled SARS-COV2 viruses inspire this NP design, and the similar Spike-related receptors between humans and hamsters motivate the adoption of hamsters as proof-of-concept. The 20-nm gold core enables the dissection of bio-nano interactions in the lungs and naturally inhibits p38α mitogen-activated protein kinase (MAPK) phosphorylation and polo-like kinase 3 (PLK3) without using chemical or biological drugs. The 20,000-Da PEG strands endow the NP with stability upon aerosolization and in bronchoalveolar lavage fluid (BALF), chemically link the RBD and gold core, and keep large sizes of the overall NP (for example, about 90 nm) to inhibit entry to systemic circulation by penetrating the air-blood barrier.

The spike RBD enables fast and abundant entry into lung epithelial cells that overexpress angiotensin-converting enzyme 2 (ACE2) and liver/lymph node-specific intercellular adhesion molecule-3-grabbing integrin (L-SIGN), both Spike RBD receptors transiently activated in hamsters upon ARDS induction. Upon repeated inhalations by Syrian hamsters with lipopolysaccharides (LPS)-induction, the NPs deposit more abundantly in ARDS lungs than in healthy lungs, effectively reducing tissue injury, oxidative stress, and inflammation in BALF cells and lung tissue more than corticosteroids. Additionally, they inhibit ARDS-related proteins without causing gold retention in major organs or toxicity 1-year post-inhalation. Similar efficacy is observed in ARDS hamsters with hydrochloric acid (HCl)-induction.

### Methods

### Study Design

The objective of this study was to develop a non-invasive, epithelium-targeted gold nanomedicine that rapidly enters lung cells with transiently activated Spike-related receptors, ultimately treating ARDS more effectively than conventional steroids without long-term systemic toxicity and accumulation. The animal models chosen were the clinically relevant LPS- or HCl-induced hamsters^{[16,20]}. All procedures followed the guidelines stipulated by the Animal Experimentation Ethics Committee (AEEC) at The Chinese University of Hong Kong (CUHK), with Approval number: 21-098-MIS. The anti-inflammatory efficacy was measured by three outcomes: ARDS-related cytokines in BALF supernatant and lavaged lung tissues, ROS level in BALF cells, and degree of tissue repair. For efficacy studies, the size of treatment group was estimated based on Dunnett's formalism and power calculation (α=0.05, power=0.80). Briefly, μ=√N δ/σ, where μ is the correlation coefficient that depends on the size of each group, N. With four different treatment groups, μ is 4.46^{[59]}. If the superior treatment group gives an outcome (δ) of 1.5 standard deviation (σ) better than the control group, the required size of N is (4.46÷1.5)²=8.84, or equivalently 9 mice per group. Hamsters were randomly assigned to each group. All in vivo mouse studies were performed in compliance with best practices and are described in the “Animal models of acute respiratory distress syndrome” and “In vivo inhalation of NPs” sections. All experiments in this study were conducted with 2-3 independent biological replicates as specified in the figure legends. The researchers were not blinded to the identity of the analysis of histological images. Blinded approach was used for blood chemistry tests.

### Data Processing and Statistical Analysis

The Prism (GraphPad Software) software and ImageJ were used for data analysis and graph construction. Statistical analysis was indicated under the figures. To determine the statistical significance in the comparison of two groups, an unpaired two-tail t test was performed. To determine the statistical significance in the comparison of multiple groups, an unpaired one-way analysis of variance (ANOVA) was performed with Tukey's Test for post hoc analysis. To determine the statistical significance in the comparison of multiple groups with 2 variables (for example, NP treatment and time), two-way ANOVA with Tukey's Test for post hoc analysis. Normality of sampling distribution of means was validated by Shapiro-Wilk test. Homogeneity of variance was validated by Bartlett's test. Results are considered significant at P<0.05.

### Preparation of Spike RBD Proteins

### Step 1: Preparation of Plasmids

Plasmids encoding His-tagged RBD (pET11a-RBD-8×His) were transformed using the heat-shock method. In particular, 50 μL of DH5α competent cells (Invitrogen) were mixed with 5 ng of plasmid, incubated on ice for 30 minutes, heat-shocked at 42° C. for 90 seconds, and incubated on ice for another 2 minutes. After adding 950 μL of pre-warmed 2% lysogeny broth (LB, Sigma, L3022), the cells were incubated for 1 hour at 37° C. with orbital shaking at 225 rpm. Then, 100 μL of the transformed cells were spread on an LB-agar plate containing 100 μg/mL ampicillin (J&K Chemical, 947040) and incubated at 37° C. overnight. One colony from each plate was picked and grown in 500 mL of LB/ampicillin (100 μg/mL) for 16 hours at 37° C. under shaking at 225 rpm. After collecting the cells by centrifugation at 3600×g for 15 minutes, the plasmids were purified using the Qiagen Plasmid Midiprep Kit per the supplier's protocol. In one embodiment, a yield of 1 μg of plasmid is obtained from 200 mL bacterial cultures.

### Step 2: Plasmids Transformation

After thawing the competent cells (previously stored at −80° C.) on ice, 100 ng of plasmids were added to 100 μL of competent cells. The cells were rested on ice for 30 minutes, heat-shocked in a 42° C. water bath for 90 seconds and incubated again on ice for 3 minutes. After adding 400 μL of fresh LB medium, the mixture was shaken at 270 rpm at 37° C. for 1 hour and centrifuged at 5000×g for 30 seconds. After discarding 400 μL of supernatant, the remaining 100 μL of pelleted cells were resuspended. The bacteria were seeded onto an LB agar plate with appropriate antibiotics and incubated at 37° C. overnight.

### Step 3: Protein Expression Induction

Expression strain Origami B cells (Sigma; 70836) pre-transformed with molecular chaperone plasmid pG-KJE8 (Takara; 3340) were transformed by RBD expressing plasmid pET11a-RBD and cultured on LB agar plate for overnight. At next noon, one single colony was picked and added into 20 mL of fresh LB medium with appropriate antibiotics. On the third day, the 20 mL of overnight cultures were added into 400 mL of fresh LB medium and continued to culture until the OD600 reaches about 0.5 (usually within about 3 h). Induction was performed by adding 0.4 mL of induction buffer containing 1 mM isopropyl β-D-1-thiogalactopyranoside (IPTG; J&K Scientific; 266729) and 4 mg/mL L-arabinose (TCI; A0515). After 6 hours, the cells were collected by centrifugation (4200×g, 15 min) for purification or storage at −80° C.

### Step 4: Protein Purification

Origami B cells from the previous step were resuspended in 40 mL of Protein Purification Buffer (20 mM Tris, 300 mM NaCl, 10% glycerol, pH=8.0) and lysed by an ultrasonication processor. After centrifuging the lysate at 15000 g, 10 minutes, the supernatant was collected, filtered by a 0.45 μm syringe filter, incubated with 3 mL of Ni-NTA resin (Promega, V8821) at RT for 1 hour, and finally transferred (together with the resin) to a blank gravity chromatography column. After discarding the liquid flow-through from the lysate-resin mixture, 18 mL of Washing Buffer (Protein Purification Buffer supplemented with 50 mM imidazole) was added to elute nonspecific binding proteins. Lastly, the RBD protein product was eluted by adding 18 mL of Elute Buffer (Protein Purification Buffer supplemented with 300 mM imidazole) and dialyzed against Storage Buffer (20 mM Tris, 300 mM NaCl, 50% glycerol, pH=8.0) for 3 times at 4° C. The purity and concentration of the protein product was determined by sodium dodecyl sulfate (SDS)-polyacrylamide gel electrophoresis (PAGE) and Bradford assay, respectively.

### Preparation of Spike RBD-Conjugated Gold Nanoparticles (Au@PEG-RBD NP)

Citrate-capped gold NPs (cit-AuNPs) having a diameter of, for example, 20 nm were synthesized using the Turkevich's method⁴⁶. After bringing 95 mL of HAuCl₄ (0.267 mM) (Sigma) to a boiling state, 5 mL of sodium citrate (1% w/v) (Alfa Aesar) was added under vigorous stirring and the mixture was kept boiling for 15 minutes. The product was slowly cooled down to room temperature (RT) to yield 20 nm cit-AuNPs. Freshly dissolved thiol (HS)-PEG₂₀ₖ-methoxy or HS-PEG₂₀ₖ-nitrilotriacetic acid (NTA) (Biochempeg), at a 1:1 molar ratio, was added to 20 nm cit-AuNPs solution at a total concentration of 5 PEG molecules per nm² of NP surface, followed by stirring the mixture at RT overnight. After adding 5 mL of NiCl₂ (50 mM) (TCI), the mixture was stirred for 1 hour to yield Au@PEG-NTA-Ni²⁺ NPs. One hundred μg of His-tagged RBD proteins were added to the Au@PEG-NTA-Ni²⁺ NP solution under stirring at 4° C. overnight to yield Au@PEG-RBD NPs. NPs were dialyzed against Nanopure water by centrifugal filtration, repeated 3 times (Thermo Fisher Scientific; MWCO 50 k).

According to the embodiments of the subject invention, the overall size of inhaled nanoparticles may be greater than 50 nm, and the core (cit-AuNP) may be in a range of 3 nm-50 nm.

To prepare untargeted NPs as control, 20-nm cit-AuNPs were reacted with only HS-PEG₂₀ₖ-methoxy and later purified by centrifugal filtration as described above, shown by FIG. 14.

### Physicochemical Characterization of NPs

### Part 1: NP Sizes and Surface Charges

In solution, the concentration of gold NPs was determined by UV-vis-NIR spectroscopy (Agilent, Cary 5000) based on the Beer-Lambert's law and the molar extinction coefficient of 20-nm AuNPs at 450 nm (5.41×10⁸ M⁻¹ cm⁻¹)⁴⁷. Hydrodynamic diameters (HD) and ζ-potentials were measured by the DelsaMax PRO light-scattering analyzer (Beckman Coulter). For HD measurements, NPs were suspended in Nanopure water or bronchoalveolar lavage fluid (BALF) freshly collected from Syrian hamsters. To test their colloidal stability, NPs were suspended in fresh BALF and incubated at 37° C. for up to 24 hours before taking HD measurements. For-potential analysis, NPs were suspended in 1 mM KCl. Reported values represent mean±SD from three independent measurements, with size histograms of NPs made from three different batches over three months to ensure batch-to-batch consistency and data reproducibility. The AuNPs were visualized by TEM at a voltage of 100 kV (Hitachi, H7700).

To characterize the morphology and measure the physical diameter of the entire PEG-coated gold NP (Au core+PEG), NPs were negatively stained for TEM imaging. In brief, 10 μL of NP solution was dropped onto a plasma-treated (Harrick Plasma), formvar/carbon-coated copper grid (200 mesh; Beijing Zhongjingkeyi Technology) and left for 30 min. The NP droplet was drawn off from the edge of the grid with a filter paper. EM Stainer solution (Nisshin-EM^{[62]}, 336) was diluted with Nanopure water by 4 times, and 10 μL of the diluted solution was added to each TEM grid for another 8 min. (EM Stainer is an electronic stain alternative to uranyl acetate.) After removing the EM Stainer solution, the grid was dried at RT for 4 h before visualization under TEM at a voltage of 100 kV (Hitachi H7700), at a magnification of 30-50 k.

Upon aerosolization, NPs were characterized real-time. NP number concentrations and aerodynamic size distributions were continuously monitored by a condensation particle counter (TSI, CPC 3775) and a scanning mobility particle sizer (SMPS) (TSI, Classifier 3082), respectively.

### Part 2: Loading of PEG Strands, Validation of Ligand Conjugation, and Ligand Stability in Biofluid

The loading of PEG strands on the AuNP was determined by thiol depletion. In particular, 1 mL of 2 nM cit-AuNPs was used to prepare Au@PEG-RBD NPs, with the unreacted free PEG strands collected, lyophilized and resuspended in 60 μL of Nanopure water. Next, 20 μL of concentrated PEG sample was mixed with 100 μL of Ellman's assay buffer [1 mM EDTA (Sigma) in 0.1 mM Na₂HPO₄ (Sigma); pH=8] and further mixed with 50 μL of Ellman's detection buffer [0.5 mg/mL of Ellman's reagent (5,5-dithiobis(2-nitrobenzoic acid)) (J&K Scientific) in the assay buffer]. After 10 minutes, the absorbance of the reaction mixture was read at 412 nm by a Multiskan GO UV-absorbance microplate reader (Thermo Fisher Scientific). The concentration of PEG was calculated with reference to a standard calibration curve after subtracting the background absorbance of the sample derived from that of the negative control. Reported data represent mean±SD from three independent experiments.

To prove the conjugation of Spike RBD proteins on the gold core, 1 mL of freshly prepared Au@PEG-RBD NPs was centrifuged (15000×g) at 4° C. for 30 min and resuspended in 1 mL of 10 mM Tris-HCl buffer (pH=8). Next, NPs were centrifuged again and resuspended in 1 mL of 1 M imidazole to release the bound proteins. The supernatant samples from each step were subject to western blot analysis to verify the release of Spike RBD proteins from the Au@PEG NP (see schematic diagram in FIG. 14). In addition, the number of conjugated RBD proteins was determined by customized ELISA.

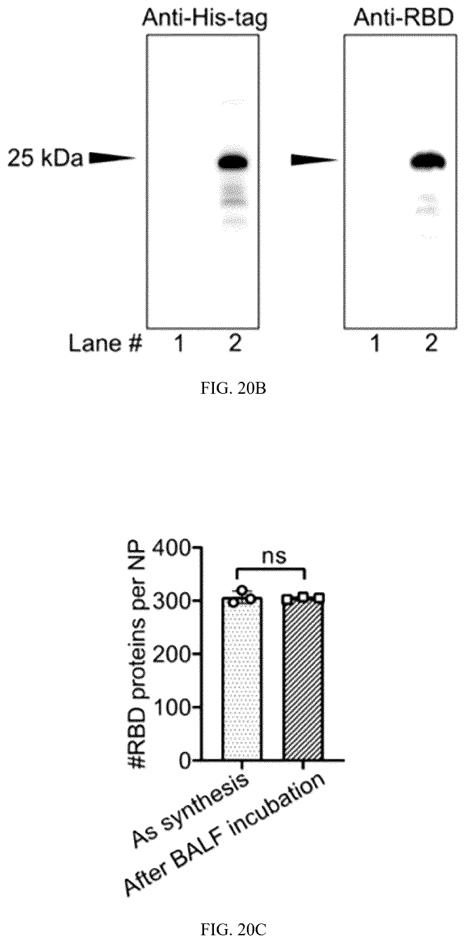

To test the extent of ligand shedding in BALF, freshly synthesized Au@PEG-RBD NPs were centrifuged (15000×g) at 4° C. for 30 minutes and resuspended by 1 mL of BALF extracted from healthy Syrian hamsters. After incubation at 37° C. for 48 hours, the mixture was centrifuged and resuspended in 1 mL of 1 M imidazole to release the bound proteins. Lastly, the NP solution was centrifuged to separate the isolated proteins and the gold NP. The NP-treated BALF before imidazole addition and the supernatant from each subsequent centrifugation steps after imidazole addition was subjected to Western blot and our in-house ELISA to analyze the ligand stability in BALF (see schematic diagram in FIG. 20).

### Part 3: Binding of NPs to Recombinant RBD Receptors (Modified ELISA)

Following a published method [Yang et al, small, 2016], 0.1 mL of recombinant ACE2 (10108-H08H-B), L-SIGN (10559-H01H), DC-SIGN (10200-H01H), mannose receptor (16065-H08H1), or MGL proteins (10821-H01H) (10 μg/mL; Sino Biological) formulated in diluent buffer (20 mM Tris, 140 mM NaCl, 10% glycerol, pH=8) was added into a 96-well ELISA plate for overnight incubation at 4° C. After three rinses with washing buffer (0.25 mL, 0.05% Tween 20 in PBS), 0.2 mL of blocking buffer (5% milk in PBS) was added into each well, and the plate was shaken for 1 hour. After rinsing, 0.1 mL of NPs (0.2 nM), formulated in diluent buffer, was added into the well. After incubation for 2 hours at RT, the wells were rinsed for 3 times. Finally, 0.1 mL of aqua regia was added to the wells, and the bound gold content in the acid solution was quantified by ICP-MS.

On the ligand competition assay, we employed the similar modified ELISA. To each well of a 96-well ELISA plate, 100 μL of 10 μg/mL recombinant Spike receptor proteins [for example, ACE2 (Sino Biological, 10108-H02H) or L-SIGN (Sino Biological, 10559-H01H)], formulated in diluent buffer (for example, 20 mM Tris, 140 mM NaCl, 10% glycerol, pH=8), was added for overnight incubation at 4° C. After three rinses with washing buffer [for example, 0.25 mL, 0.05% Tween 20 in phosphate-buffered saline (PBS)], 0.2 mL of blocking buffer (for example, 5% milk in PBS) was added into each well, and the plate was shaken for 1 hour. After three rinses, 0.2 mL of diluent buffer containing 3 nM NPs and free recombinant Spike RBD proteins at different concentrations (for example, up to 8 μM) were added for incubation for 2 hours at RT. After three rinses, 0.1 mL of aqua regia was added, and the bound gold content in solution was quantified by ICP-MS.

Part 4: Leakage of Nickel Ions from Au@PEG-RBD NP

Two mL of the freshly synthesized Au@PEG-RBD NP product in the dialysis bag was collected for tracing the nickel concentration in the solvent. Briefly, 1 mL of the dialysis product was centrifuged at 8000×g 10 minutes to harvest the supernatant for ICP-MS detection. Another 1 mL of the dialysis product was stored at 4° C. After 1 month, the NP solution was centrifuged at 8000×g for 10 minutes, and the supernatant was collected for ICP-MS detection. The nickel concentration of these two samples was compared to detect possible nickel leakage.

### Part 5: Cytotoxicity of NP

A549 alveolar epithelial cancer cells (ATCC; CCL-185) and HEK293 cells (ATCC, CRL-1573) were cultured in complete Dulbecco's Modified Eagle Medium [DMEM supplemented with 10% fetal bovine serum (Gibco, A5256701) and 1% penicillin/streptomycin (Gibco, 15070063)]. BEAS-2B cells (non-cancerous bronchial epithelial cells; ATCC, CRL-3588) were cultured in LHC-8 medium (Gibco, 12678017). Cells were seeded in 96-well plates at a density of 1000 cells/well. After 12 hours, the medium was switched to fresh medium containing different concentrations of Au@PEG-methoxy and Au@PEG-RBD NPs (up to 6 nM, 100 μL/well) and incubated for three more days. After two PBS rinses, cell viability was measured by the alamarBlue assay (Invitrogen, DAL1025) according to the manufacturer's protocol.

### Protein Corona Composition of NPs

### Part 1: Collection of BALF Proteins Adsorbed to NPs

In one embodiment, 0.1 mL of concentrated NPs (300 nM) was incubated in 0.9 mL of fresh BALF collected from Syrian hamsters (male, 8 weeks-old) for 30 minutes at 37° C. The protein-adsorbed NPs were washed to remove unbounded proteins by three rounds of centrifugation (13500 rpm at 4° C.) and resuspended in PBS containing 0.05% v/v Tween 20 (Sigma). Equal volumes of BALF without NPs were treated as a control for nonspecific protein adsorption. Upon centrifugation of the washed NPs, the supernatant was discarded and 25 μL of the remaining NP pellet was treated with 10 μL of Laemmli sample buffer (4×) (Bio-Rad) and 5 μL of 4.8 M dithiothreitol (J&K Chemicals; 415951). Upon incubation at 70° C. for 1 hour with shaking to release the adsorbed proteins, the NPs were removed by centrifugation at 13500 rpm at 4° C., and the supernatant containing desorbed proteins were characterized by PAGE, purified, and quantified based on literature precedent.

### Part 2: PAGE

Mercaptoethanol (0.5 μL) (Sigma) was added to 40 μL of the desorbed proteins in Laemmli sample buffer. After heating the mixture at 95° C. with shaking for 5 minutes, 6 μL of denatured proteins and 6 μL of Precision Plus Protein Dual Color Standards (Bio-Rad) were loaded onto a 4-20% Mini-PROTEAN TGX Stain-Free Gel (Bio-Rad) in tris-glycine SDS running buffer (Bio-Rad). After resolution by electrophoresis at 200 V for 40 minutes, the bands were visualized by the ChemiDoc Touch gel imaging system (Bio-Rad).

### Part 3: Label-Free Proteomics Analysis

Liquid chromatography tandem mass spectrometry (LC-MS/MS): All reagents used were chromatography grade (Thermo Fisher Scientific). Extracted peptides were lyophilized and resuspended in 10 μL of 0.1% formic acid for separation using an Orbitrap Eclipse Mass Spectrometer (Thermo Fisher Scientific), equipped with a 150 μm×15 cm in-house made column packed with Acclaim PepMap RPLC C18 (1.9 μm, 100 Å, Dr. Maisch GmbH). The organic gradient was driven by the Nanoflow UPLC system over 120 minutes using Buffer A (0.1% formic acid in water) and Buffer B (20% 0.1% formic acid in water and 80% acetonitrile) at a flow rate of 600 nL/min. The gradient was held from 4% to 8% B for 3 minutes, from 8% to 28% B for 86 minutes, from 28% to 40% B for 20 minutes, from 40% to 95% B for 1 minute, and from 95% to 95% B for 10 minutes. Eluted peptides were directly sprayed into the mass spectrometer. Ten MS/MS data-dependent scans were acquired simultaneously with one high-resolution (60000 at 400 m/z) full-scan mass spectrum to provide the amino acid sequence and mass-to-charge ratio for the selected peptide ions.

### Cellular Uptake

### Part 1: ICP-MS

HEK293 cells were seeded in a confocal dish or 24-well plate at a density of 0.5×10⁶ cells per well for 12 hours before transfection. Next, 0.3 mL of transfection medium was added to each well. The transfection medium for each well contains 1.5 μL of Lipofectamine 2000 (Invitrogen) and 500 ng of plasmid DNA (either pcDNA3.1-ACE2-GFP, pCMV6-L-SIGN-GFP or pCMV-CD81-GFP) formulated in OptiMEM. After 12 hours of transfection, the transfection medium was switched to DMEM supplemented with 10% FBS and 1% PS. Next, cells were incubated with 2 mL of 0.2 nM NPs (in complete culture medium) for 2 hours. After two rinses with phosphate-buffered saline (PBS), cells were harvested by adding 0.5 mL aqua regia to the wells. The dissolved content was diluted 10 times by Nanopure water and filtered by 0.2 μm filter before subjecting to ICP-MS (Agilent 7900) measurements.

### Part 2: Time-Dependent Cellular Uptake

Upon transfection with pcDNA3.1-ACE2-GFP or pCMV6-L-SIGN-GFP as described in Part 1, cells were incubated with 2 mL of 0.2 nM NPs (in complete culture medium) for various time durations up to 24 h. After two rinses with PBS, cells were harvested by adding 0.5 mL of aqua regia to the wells. The dissolved content was diluted 10 times by Nanopure water and filtered through a 0.2 μm filter before ICP-MS (Agilent 7900) measurements.

### Part 3: Confocal Microscopy

HEK293 cells (ATCC) were transfected with human ACE2-GFP (Addgene, 154962), pCMV6-L-SIGN-GFP (OriGene; RG226741), or mPA-GFP-CD81-10 (Addgene; 57124) by Lipofectamine 2000 to overexpress the fluorescent proteins above. Next, cells were incubated with 2 mL of 0.2 nM Cy5.5-labelled NPs (in complete culture medium) for 2 hours. After two rinses with phosphate-buffered saline (PBS) twice, cells were fixed by 1 mL of 4% paraformaldehyde for 20 minutes and stained by 1 mL of 1 μg/mL DAPI (4′,6-diamidino-2-phenylindole; Thermo Fisher Scientific) for 10 minutes at RT for imaging under a Leica SP8 confocal microscope. The excitation wavelengths of DAPI, GFP, and Cy5.5 are 405 nm, 488 nm, and 651 nm, respectively. The emission wavelength ranges of DAPI, GFP, and Cy5.5 are 415-500 nm, 500-520 nm, and 670-790 nm, respectively.

### Animal Models of Acute Respiratory Distress Syndrome (ARDS)

All procedures followed the guidelines stipulated by the Animal Experimentation Ethics Committee (AEEC) at The Chinese University of Hong Kong (CUHK). Male Syrian hamsters between 8 and 12 weeks of age were randomly divided into various treatment groups and housed in a temperature- and humidity-controlled environment with a 12-h light/dark cycle at the Laboratory Animal Services Centre (LASEC). Before establishing acute respiratory distress syndrome (ARDS) models, hamsters were anesthetized by an i.p. injection of about 0.5 mL of ketamine/xylazine (150 mg/kg and 10 mg/kg body weight, respectively). For lipopolysaccharide (LPS)-induced ARDS models, 120 μL of 12.5 mg/mL LPS (Santa Cruz; sc-221855B) was intranasally injected into a ˜150 g hamster (10 mg LPS/kg body weight). For HCl-induced ARDS models, 120 μL of HCl (pH=2) was intranasally injected into a ˜150 g hamster (800 μL/kg body weight).⁴⁸ After monitoring the breath and heartbeat for 10 minutes post-induction, the hamsters were returned to the cages for 12 hours until inhalation studies. Hamsters were provided with food and water when they were not placed in inhalation chambers.

### Customized ELISA for Detecting Spike RBD Proteins and Spike Receptors

The protocols below were adapted from our past work^{[21]}. As-synthesized recombinant RBD proteins or Spike receptors [ACE2 (Sino Biological, 10108-H02H), L-SIGN (Sino Biological, 10559-H01H), DC-SIGN (Sino Biological, 10200-H01H), mannose receptor (LS Bio, LS-G20979-20), or MGL (Sino Biological, 10821-H01H) proteins] of certain concentrations (up to 10 μg/mL, for plotting standard curves) or samples, all formulated in diluent buffer (20 mM Tris, 140 mM NaCl, 10% glycerol, pH=8), was added into a 96-well ELISA plate for overnight incubation at 4° C. After three rinses with washing buffer [0.25 mL, 0.05% Tween 20 in phosphate-buffered saline (PBS)], 0.2 mL of blocking buffer (5% milk in PBS) was added into each well, and the plate was shaken for 1 h. After rinsing, 100 μL of diluent buffer containing primary antibodies [2 μg/mL for RBD (R&D, MAB10540), 2 μg/mL for ACE2 (Proteintech, 66699-1-Ig), 40 μg/mL for DC-SIGN (Invitrogen, MA5-35828), 20 μg/mL for L-SIGN (Invitrogen, PA5-68454), 5 μg/mL for mannose receptor (Invitrogen, PA5-46994), or 3 μg/mL for MGL (Invitrogen, PA5-82781)] were added into each well and incubated at RT for 1 h. After rinsing, 100 μL of horse radish peroxidase (HRP)-conjugated secondary antibody (1 μg/mL in diluent buffer; Invitrogen, 31460) for 1 hour at RT. After rinsing, 100 μL of 3,3′,5,5′-tetramethylbenzidine (TMB; Bethyl, E102) was added into wells and incubated for 15 min at RT in the dark. Finally, 100 μL of 2 N H₂SO₄ was added to stop the reaction. The absorbance was read at 450 nm and 570 nm by a microplate reader.

### Ex Vivo Binding of NPs to Lungs

Hamsters were sacrificed and placed on the operation table. After making a 2-cm skin incision horizontally at the neck, the fascia above the mandibular gland and muscles was dissected. After further removing the mandibular gland and muscles to reveal the trachea, a small opening was made at the up opening of trachea. Then, a soft rubber tube [modified from a BD Vacutainer® Safety-Lok™ blood collection needle (BD; 367292) by removing metal needles of the ends] was inserted into the trachea. After tying the rubber tube and trachea together by a suture, 2 mL of 0.2 nM NPs in water were injected into the lungs through the tube. After 30 minutes of incubation, the NPs were aspirated, and the lungs were washed with 2 mL of PBS for three times using injection and aspiration cycles, and finally dissected and immersed in aqua regia for ICP/MS analysis or 4% PFA for tissue sectioning.

### In Vivo Inhalation of NPs to the Lungs

Below, we denote “100%” as the lung gold content in the case of “full receptor targeting” achievable by the targeted Au@PEG-RBD NP without any receptor blocking and “0%” in the case of “no receptor targeting” achievable by the untargeted Au@PEG-methoxy NP.

### Part 1: Binding to the Lungs Via L-SIGN and ACE2

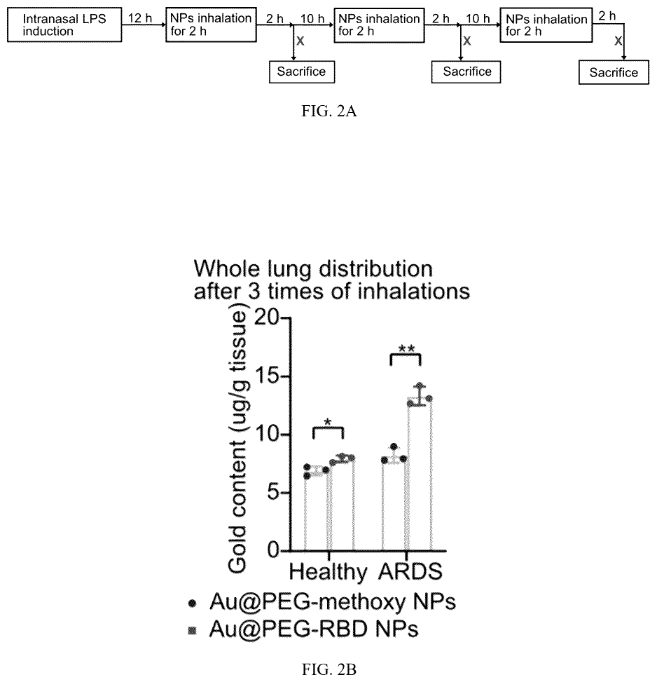

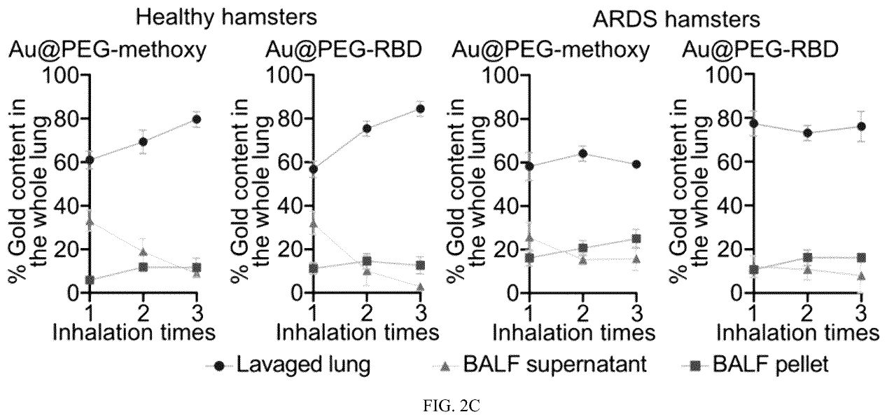

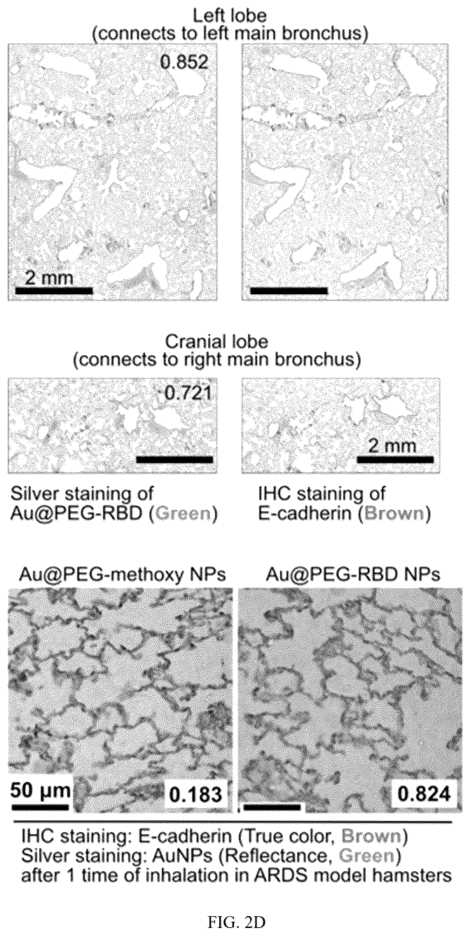

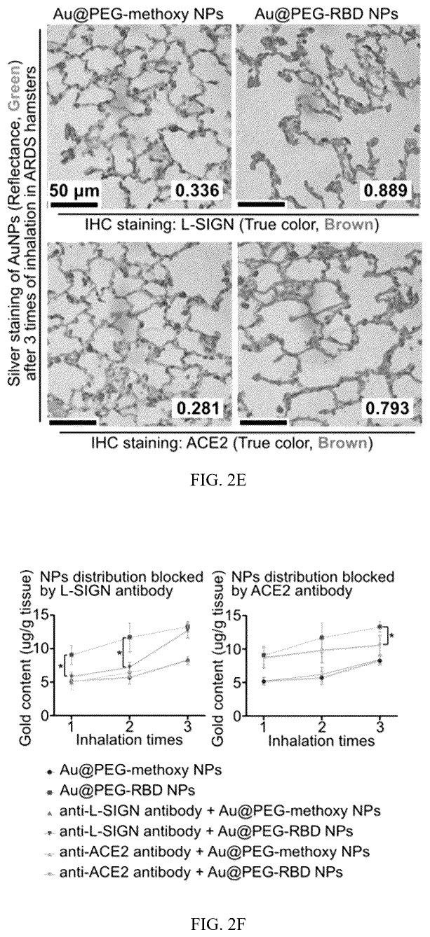

Based on our dose regimen as shown in FIG. 2A, LPS-induced ARDS hamsters inhaled NPs for 2 hour and were sacrificed immediately or returned to the holding cage for 10 hours, with this iteration lasting till the third inhalation. To address whether Au@PEG-RBD NP specifically bound to the lungs via L-SIGN or ACE2 in vivo and to assess the relative contribution of each Spike receptor in lung delivery, hamsters underwent an additional antibody blocking step prior to each round of NP inhalation. At the designated terminal timepoint, hamsters (i) were anesthetized by an i.p. injection of normal saline containing ketamine (150 mg/kg-hamster) and xylazine (10 mg/kg-hamster) 1 hour prior to NP inhalation, (ii) were intranasally injected with 100 μL of PBS containing primary antibodies against L-SIGN (20 μg/mL; Invitrogen, PA5-68454) or ACE2 (20 μg/mL; Proteintech, 66699-1-Ig) and returned to the cage for 1 hour, (iii) received inhalation of 6 nM Au@PEG-RBD NPs for 2 hours, and (iv) were finally sacrificed for dissecting the lungs. A pronounced reduction in gold contents in the lungs (as measured by ICP-MS) indicates specific binding of Au@PEG-RBD NP to L-SIGN or ACE2 and a prominent role of the Spike receptor in mediating lung accumulation.

[Au content (Au@PEG-RBD NP)−Au content (Au@PEG-RBD NP with single blocking)]=[Au content (Au@PEG-RBD NP)−Au content (Au@PEG-methoxy NP)]×100%

### Part 2: Relationship Between L-SIGN and ACE2 in Mediating Lung Delivery

To assess the additive or synergistic role of both Spike receptors in lung delivery, we assessed how affect the lung accumulation of a single dose of Au@PEG-RBD NP at different stages of ARDS onset. LPS-induced ARDS hamsters underwent single blocking of L-SIGN, single blocking of ACE2, or double antibody blocking of both receptors prior to NP inhalation. Upon anesthesia, hamsters were intranasally injected with 100 μL of PBS containing primary antibodies against L-SIGN (20 μg/mL), ACE2 (20 μg/mL), or both antibodies for 1 hour. Other inhalation and animal sacrifice procedures were identical. The combined contributions of both receptors (%) were calculated using the formula below. Both receptors mediate NP delivery to the lung additively if the combined contribution of both receptors equals the sum of contribution of each receptor. Both receptors mediate NP delivery synergistically if the combined contribution of both receptors exceeds the sum of contribution of each receptor.

[Au content (Au@PEG-RBD NP)−Au content (Au@PEG-RBD NP with double blocking)]=[Au content (Au@PEG-RBD NP)−Au content (Au@PEG-methoxy NP)]×100%

NPs (6 nM in Nanopure water) were aerosolized by an aerosol generator (Classifier 3076, TSI) by flowing compressed air (Linde Industrial Gases) at a rate of 3 L/min. Airborne NPs mixed with compressed air were passed through a diffusion dryer (TSI, 3062-NC) at a flow rate of 3 L/min. Syrian hamsters (n=3), placed inside an airtight whole-body inhalation chamber (Scivenas Cientific, RES644), were exposed to airborne NPs at a constant concentration of 9×10⁶ NP/cm³ for 2 hours. Excess airborne NPs were cleared by aerosol filters (Bio-Gene Technology), and the connecting tube (with an inner diameter of 0.19 in., TSI) from the outlet of filter to the exhaust was soaked in water. The flow rates at the inlet (3.0 L/min) and outlet (>2.9 L/min) of the chamber were continuously monitored by a mass flow meter (TSI 4140) to ensure limited leakage of airborne NPs. After inhalation, hamsters were sacrificed immediately or returned to the animal cage for further studies. Hamsters were sacrificed by overdose of CO₂ for collection of BALF, the lavaged lung tissue, and other organs. BALF was extracted in situ by infusing the whole lung with 0.5 mL of PBS for three times.

### In Vivo Blocking of RBD

ARDS hamsters were anesthetized by an i.p. injection of ketamine/xylazine (150 mg/kg and 10 mg/kg body weight, respectively) 1 hour prior to inhalation. After intranasally injecting 100 μL of primary antibodies against L-SIGN (20 μg/mL; Invitrogen, PA5-68454) or ACE2 (2 μg/mL; Proteintech, 66699-1-Ig), the hamsters were put back to cages for 1 hour and then received inhalation of 6 nM NPs for 2 hours. Reduction in gold content in the lungs indicates blocking of the L-SIGN or ACE2 by RBD.

### Immunohistochemistry (IHC)

Tissues were fixed in 10% buffered formalin (3.7% w/v) for 48 hours and stored in PBS (0.1 M, pH=7.5) at 4° C. Fixed tissues were dehydrated in ethanol, cleared in xylene, and embedded in paraffin blocks. Paraffin-embedded tissue sections (4 μm) were cut and mounted on Superfrost Plus™ Adhesion microscope slides (Thermo Scientific). IHC staining was performed on paraffin sections with antigen retrieval. Tissue sections were deparaffinized and rehydrated. After placing slides in citrate buffer (10 mM citric acid, pH=6), the slides were heated in the microwave oven for 3 minutes under high power (at about 95-100° C.) and for 20 more minutes under low power. After cooling the slides in the heated solution for 30 min, they were rinsed in distilled water twice and in PBS for 5 minutes. The slides were blocked with 2.5% normal horse serum (Vector Laboratories) for 2 hours and incubated with 60 μL of primary antibodies [2 μg/mL for ACE2 (Proteintech, 66699-1-Ig), 40 μg/mL for DC-SIGN (Invitrogen, MA5-35828) 20 μg/mL for L-SIGN (Invitrogen, PA5-68454), 5 μg/mL for mannose receptor (Invitrogen, PA5-46994), or 3 μg/mL for MGL (Invitrogen, PA5-82781)] at 4° C. overnight. Slides were washed in PBS, treated with 3% H₂O₂ (Merck Millipore) for 30 min, rinsed, and incubated with 50 μL of secondary antibodies (ImmPRESS HRP Polymer Detection Kit, Vector Laboratories) for 30 minutes. The sections were developed sequentially using 3,3′-diaminobenzidine (DAB) enzyme substrate (ImmPACT™ DAB, Vector Laboratories) for 2 minutes. Slides were counterstained with Mayer's hematoxylin for 3 minutes, washed in distilled water, dried in 90% ethanol, and mounted with xylene-based mounting medium (DPX Mountant; Sigma, 06522). Bright-field images were taken with a Nikon Eclipse Ni (DS-Ri2) microscope.

### Organ-Level Distribution by ICP-MS

Organs were excised from the hamsters, minced, and fully digested in 1 mL of aqua regia overnight at RT. Blood, BALF supernatant, or BALF cell pellets (0.3 mL) were digested by 0.25 mL of aqua regia. The lysate was diluted to 2% HNO₃ by adding Nanopure water with 10 ppb indium as internal standard, followed by passing through a 0.2 μm hydrophilic syringe filter for ICP-MS measurements (Agilent 7900).

### Tissue-Level Distribution

### Part 1: Silver Enhancement Staining

Paraffin-embedded lung tissue sections of 4 μm thick were deparaffinized in xylene (5 min×3 times) and rehydrated through a series of ethanol (100%, 90%, 70%; 3 min×2 times at each ethanol concentration), and Nanopure water (5 min×5 times). The rehydrated tissue sections were stained by the Silver Enhancement Kit for Light and Electron Microscopy (Ted Pella). The silver enhancement solutions, Solution A (silver salt) and Solution B (initiator), were mixed at a 1:1 ratio right before use. A drop of the mixture (˜50 μL) was applied to the tissue section for 20 minutes under normal laboratory lighting. Next, the tissue sections were rinsed with Nanopure water (3 min×3 times), followed by counterstaining with Mayer's Hematoxylin (blue-purple nuclear stain; Vector Laboratories) or methyl green (blue-green nuclear stain; Vector Laboratories) for 10 minutes. Bright-field images were acquired using the Nikon Eclipse Ni (DS-Ri2) microscope.

### Part 2: Confocal Reflectance Microscopy

Confocal reflectance images of tissue sections overlaid with true color images were obtained using a Leica SP8 confocal microscope, under reflectance mode with 20× objectives under 488 nm excitation. True color images were produced by overlaying the red, green, and blue (RGB) channels in the transmitted light imaging mode.

### Cellular-Level Distribution by TEM

Tissue blocks (˜1 mm³ from the right caudal lobe) or pelleted BALF cells were fixed with 2.5% glutaraldehyde in phosphate buffer (pH=7.2-7.4) for 2 hours and stained by 1% osmium tetroxide for another 2 hours. Blocks were gradually dehydrated in increasing ethanol gradients and propylene oxide, embedded in Epon 812 resins (Electron Microscopy Sciences; EMS), and polymerized at 55° C. for 48 hours. Ultrathin sections of ˜70 nm thick were deposited onto 200-mesh copper grids (EMS) and stained with 4% uranyl acetate (EMS, in 50% methanol/water) and Reynolds lead citrate (Sigma) for observation under TEM at a beam voltage of 100 kV (Hitachi H7700).

### Anti-ARDS Efficacy

LPS- or HCl-induced ARDS hamster models were randomly divided into 6 groups [untreated, Au@PEG-methoxy NP (˜9×10⁶ NP/cm³; inhalation), Au@PEG-RBD NP (˜9×10⁶ NP/cm³; inhalation), free PEG-RBD strands (intranasal injection; 200 nM, 200 μL), L-SIGN or ACE2 antibody (intranasal injection; 20 μg/mL, 150 μL) prior to inhalation of the same concentration of Au@PEG-RBD NP, or hydrocortisone (5 mg/kg; SinoPharm, H20023069; intraperitoneal)]. 12 hours or 36 hours post-induction, various groups of hamsters received their respective treatments, while hamsters in the untreated group inhaled compressed air. Typically, BALF supernatant, BALF cells, and lavaged lung tissue were extracted from the hamster 2 hours post-administration for studying anti-ARDS efficacy at various time points (i.e., after the 1^{st}, 2^{nd}, or 3^{rd} administration). After establishing the superior treatment Au@PEG-RBD NP in the preceding study, we also included free PEG-RBD conjugate as the non-therapeutic control (inhalation, 2 μM in PBS) to enable the attribution of effects specifically to Au@PEG-RBD NP.

### Part 1: Cytokines of BALF Supernatant and Lavaged Lung Tissues

Levels of cytokines (IL-6, IL-8, TNF-α, and IFN-γ) in BALF supernatant were determined by ELISA (IL-6: FineTest, EHA0006, IL-8: Krishgen Biosystems, KLH0059, TNF-α: FineTest, EHA0004, and IFN-γ: FineTest, EHA0005, IL-4: FineTest, EHA0001, IL-10: FineTest, EHA0008) per the protocol provided by the manufacturer. Levels of cytokines in lavaged lung tissues were determined by western blot. After adding 20 μg of lavaged lung tissues to a 10% denaturing PAGE gel (BioRad) for electrophoresis, the gel was transferred to a polyvinylidene difluoride membrane (BioRad) at 100 V on ice for 2 hours. After blocking in 5% BSA (Rockland) in Tris-buffered saline-Tween (TBST) buffer for 1 hour, the blots were incubated with primary antibodies against IL-6 (Invitrogen, 701028), IL-8 (Invitrogen, AHC0881), TNF-α (Invitrogen, PA1-40281), or IFN-γ (Invitrogen, MM700B) diluted in TBST containing 5% BSA overnight at 4° C. After incubating with 1 μg/mL secondary goat antibody [conjugated with horse radish peroxidase (HRP)] against rabbit (Bio-Rad, 1706515) diluted in TBST containing 5% non-fat milk for 1 h, the membranes were treated with Clarity™ Western ECL Substrate (Bio-Rad) to visualize the protein bands using a ChemiDoc Touch Imaging System (Bio-Rad).

### Part 2: Cytokines of Lavaged Lung Tissues

Lavaged lung tissues were collected to measure the levels of cytokines by western blot. After adding 20 μg of lavaged lung tissues to a 10% denaturing PAGE gel (Bio-Rad) for electrophoresis, the gel was transferred to a polyvinylidene difluoride membrane (ThermoFisher) at 25 V, 4 A by a Power-blotter Semi-dry Transfer System (ThermoFisher) for 10 minutes. After blocking in 5% BSA (Rockland) in Tris-buffered saline-Tween (TBST) buffer for 1 hour, the blots were incubated with primary antibodies against IL-6 (Invitrogen, 701028, 0.1 μg/mL), IL-8 (Invitrogen, AHC0881, 0.1 μg/mL), TNF-α (Invitrogen, PA1-40281, 0.1 μg/mL), IFN-γ (Invitrogen, MM700B, 2 μg/mL), IL-4 (Invitrogen, PA5-115416, 1 μg/mL), or IL-10 (Invitrogen, PA5-95561, 1 μg/mL) diluted in TBST containing 5% BSA overnight at 4° C. After incubating with 1 μg/mL secondary goat antibody [conjugated with HRP] against rabbit (Bio-Rad, 1706515) diluted in TBST containing 5% non-fat milk for 1 h, the membranes were treated with Clarity™ Western ECL Substrate (Bio-Rad, 1705060) to visualize the bands using a ChemiDoc Touch Imaging System (Bio-Rad).

### Part 3: Histology

Tissue slides containing the central and lateral parts of the lungs were scored largely based on these criteria [Xiong et al, AHM, 2018]: (1) edema and hemorrhage; (2) congestion of alveolar septum; (3) cell infiltration of alveolar septum/lumina; and (4) necrosis/cellular debris of alveolar septum. Table 7 lists the grading scale. The feature “hyaline membranes formation” is not included when calculating the lung damage score because no hyaline membrane was identified in the tissue, in agreement with literature precedent [Aeffner et al, Toxicologic Pathology, 2015].

### Part 4: Reactive Oxygen Species (ROS) Measurement

Freshly extracted BALF cells were resuspended in PBS, counted by Trypan Blue, and seeded in a 96-well plate with a volume of 100 μL per well (˜3000 cells/well). Using the Cellular ROS Assay Kit (Deep Red Fluorescence) from Abcam (ab186029), 100 μL of ROS Working Solution was added into each well and incubated at 37° C. for 30 minutes. The fluorescent signal of each well was read by a microplate reader. The excitation and emission wavelengths of Deep Red are 650 nm and 675 nm, respectively.

### Part 5: Count of Immune Cells in BALF

Freshly extracted BALF cells were resuspended in 1 mL of PBS containing 2 mg/ml EDTA. Total BALF cell number was counted by Trypan blue on a hemocytometer slide. For classifying and counting different BALF cell types, the extracted cells were resuspended in 50 μL of PBS containing 2 mg/mL EDTA, dropped on to a slide to make a cell smear, stained by a Wright-Giemsa staining kit (Yeasen, 60529ES01) per manufacturer's instruction, and analyzed by a light microscope based on cell morphology^{[67]}.

### Part 6: QRT-PCR and Western Blot Analysis of Macrophage Markers

RNA was isolated using RNAiso Plus (Takara, #9108) and reverse-transcribed using the RevertAid First Strand cDNA Synthesis Kit (Thermo Scientific, K1622) to generate cDNA. qRT-PCR was performed on the StepOnePlus Real-Time PCR System (Applied Biosystems) using the TB SYBR Green Premix Ex Taq kit (Takara, RR82WR) following the manufacturer's instructions. Transcript levels were analyzed using the ΔΔCT method and normalized to the house-keeping gene glyceraldehyde 3-phosphate dehydrogenase (GAPDH). Gene expression levels were quantified using pre-designed primers purchased from Shanghai Rui Mian Bio-Tech

For western blot, the BALF cell pellet was lysed by adding 50 μL of Laemmli sample buffer (1×) (Bio-Rad, 1610747) for XX min. The electrophoresis and blotting procedures were the same as above. The primary antibodies were: CD86 (1 μg/mL, ThermoFisher, MA5-35211) and CD80 (1 μg/mL, Abcam, ab254579) for M1 phenotype, and CD206 (2 μg/mL, Proteintech, 60143-1-Ig) and arginase-1 (2 μg/mL, ThermoFisher, MA5-56598) for M2 phenotype.

### Part 7: Lung Function Measurement by Whole Body Plethysmography

Hamsters were placed in transparent Perspex whole-body plethysmography chambers (one animal per chamber; diameter, 19.1 cm; height, 14 cm; volume, 4,014.83 cm³; Data Sciences) in which the airflow at 2.5 L/min was provided by a bias flow generator (Data Sciences) and food and water were available ad libitum. Each chamber was equipped with a Validyne pressure transducer (600-900 mmHg), a temperature sensor (0-100° C.), and a humidity sensor (0-100%). All channel signals from chambers were collected using an ACQ7700 Carrier and a UniversalXE Signal Conditioner connected to a Micro 1401 data acquisition unit (Cambridge Electronic Design). Signals were thereafter acquired and analyzed using Spike2.

### Mechanism for the Anti-ARDS Efficacy of Gold NPs

### Part 1: Change in Protein Expression

The identification of the differentially expressed proteins (DEPs) was achieved by comparing the group of Au@PEG-methoxy NP-treated ARDS hamsters to the group of untreated ARDS hamsters. In the process of DEP screening, proteins with a fold change ≥2 and P<0.05 were considered as upregulated, while proteins with a fold change ≤½ and P<0.05 were considered as downregulated. Pathway mapping of DEPs was performed using the Kyoto Encyclopedia of Genes and Genomes (KEGG) database (http://www.genome.jp/kegg/), and the distribution of the proteomics data in biological process (BP), cellular components (CC), and molecular functions (MF) was obtained by mapping to the Gene Ontology database (https://geneontology.org).

### Part 2: Kinome Profiling

After verifying the stock solution in water (20 μM; 100×) by ICP-MS, Au₂₀@PEG₅₀₀₀-methoxy NPs, transferred to absolute dimethyl sulfoxide (DMSO) by dialysis, were sent for testing the activities of a library of 250 kinases using the Z′LYTE biochemical assay at SelectScreen Kinase Profiling Service (Thermo Fisher). In particular, Z′LYTE uses a fluorescence-based, coupled-enzyme format based on the differential sensitivity of phosphorylated and non-phosphorylated peptides to proteolytic cleavage. The NP sample was screened in 1% DMSO (final) in the well at a single concentration of 200 nM (1×). To calculate the IC₅₀ value of a screened high-performing kinase (with at least 80% inhibitory activity), the NP sample was subject to 10-point, 3-fold serial dilutions from the starting NP concentration of 200 nM (1×) for constructing the dose-response curve. Assays were conducted using the ATP concentration as indicated in the corresponding tables, depending on the format of detection. For “Direct Format” that operates through phosphorylation and activation of a synthetic peptide substrate for a given kinase, [ATP] was set at its apparent Kₘ value (K_{m,apparent}), as previously determined by the Z′LYTE assay. For “Cascade Format” that operates through phosphorylation and activation of the inactive downstream kinase of a given kinase, [ATP] was set at 100 μM.

### Part 3: Kinome Profiling Data Analysis

Percent phosphorylation (% Pho) was determined with reference to the 0% phosphorylation (or 100% inhibition) control (which contains no ATP and therefore exhibits no kinase activity) and the 100% phosphorylation control (which contains the phosphorylated peptide as the same sequence as the peptide substrate). Control wells do not include any kinase inhibitors. Percent inhibition values were calculated by this equation: % Inhibition=[1−% PhoNP/% Pho0% inhibition ctrl]×100, whereby the 0% inhibition control contains the active kinase. IC50 values were fitted from the dose-response curves based on model number 205 of XLfit from IDBS.

### Part 4: In Vitro Validation of Proteomic and Kinome Profiling Data

Seeded in 10-cm dishes at a density of 3×10⁶ cells per dish, non-cancer BEAS-2B bronchial epithelial cells were supplemented with 10 mL of complete DMEM. At ˜80% confluency, the medium was replaced by fresh complete DMEM supplemented with either (i) 200 μL of PBS, (ii) 200 μL PBS containing 1 mg/mL LPS, (iii) 200 μL PBS containing 1 mg/mL LPS+10 nM Au@PEG-methoxy NP, or (iv) a physical mixture of 2 mg/mL LPS (in 100 μL of PBS) and 50 μM vx-702 (in 100 μL of DMSO; MCE, HY-10401). After 6 hours of incubation, cells were washed 3 times by PBS and harvested by the Nuclear/Cytosol Fractionation Kit (Abcam, ab289882) per manufacturer's protocol. Levels of Elavl1, p-p38α, and total p38α were determined by western blot. Blots were incubated with primary antibodies against Elavl1 (Proteintech, 11910-1-AP, 0.5 g/mL), p-p38α (Invitrogen, MA5-15177, 0.1 μg/mL), total p38α (Cell Signaling, 9218, 0.1 μg/mL), or β-tubulin (Abcam, ab108342, 0.1 μg/mL). Refer to the above protocols for secondary antibody and blot imaging.

### Biocompatibility

### Part 1: Immunogenicity

After three rounds of inhalation of Au@PEG-RBD NPs, hamsters were returned to the cage for 1, 14, or 28 days. After collecting the serum or BALF and centrifuging at 4000 rpm for 10 minutes, 0.1 mL of supernatant was added to each well of a 96-well ELISA plate for overnight incubation at 4° C., using the commercial mammalian-expressed RBD protein (SinoBiological; 40592-V08H) as a positive control. After three rinses with 0.25 mL of washing buffer (0.05% Tween 20 in PBS), the plate was incubated with 0.2 mL of blocking buffer (5% milk in PBS) with shaking for 1 hour. After rinsing, 0.1 mL of 0.4 μg/mL hamster specific HRP-conjugated IgA and IgG antibodies (Brookwood Biomedical, sab3003A and sab3003G) were added into the well for incubation at RT for 2 hours. After three rinses, 0.2 mL of TMB (3,3′,5,5′-tetramethylbenzidine; Thermo Scientific; 34022) substrate was added and incubated for 30 min before the absorbance at 450 nm was detected by a microplate reader.

### Part 2: Long-Term Toxicity

After receiving inhalation of NPs for 3 consecutive days, the ARDS model hamsters were returned to the cages for 1, 6, or 12 months. At sacrifice, fresh blood samples were sent to the PathLab Clinic (Kowloon, Hong Kong) to test hematological and biochemical parameters. Major internal organs were dissected for detecting the remaining gold contents by ICP-MS and histologically examined for morphology changes in the tissue.

### Data Processing and Statistical Analysis

The Prism (GraphPad Software) software and ImageJ was used for data analysis and graph construction. Statistical analysis was indicated under the figures. Normality of sampling distribution of means was validated by Shapiro-Wilk test. Homogeneity of variance was validated by Bartlett's test. Results are considered significant at P<0.05.

### Results

### EXAMPLE 1—Au@PEG-RBD NPs AND THEIR BINDING TO ACTIVATED ACE2 AND L-SIGN

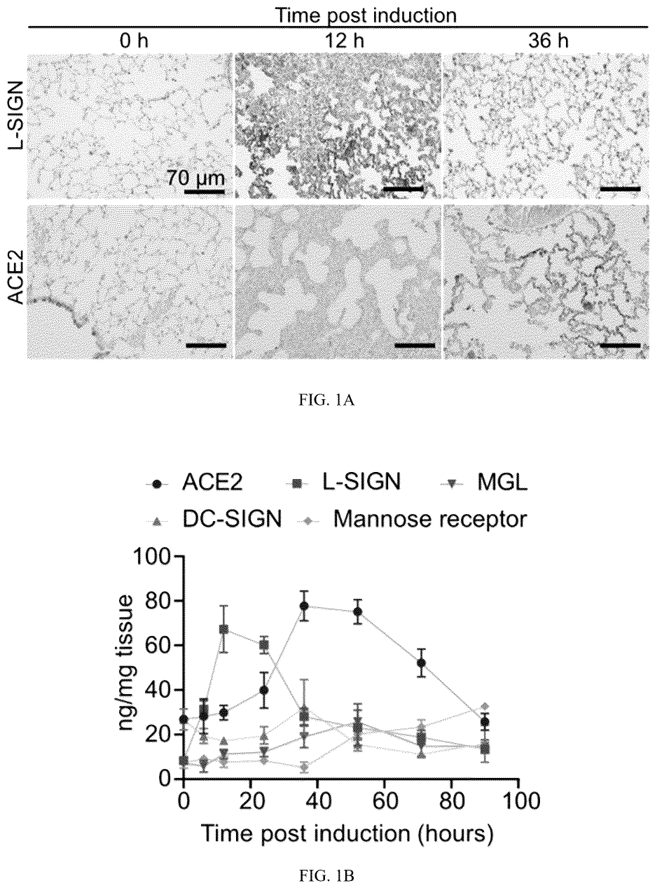

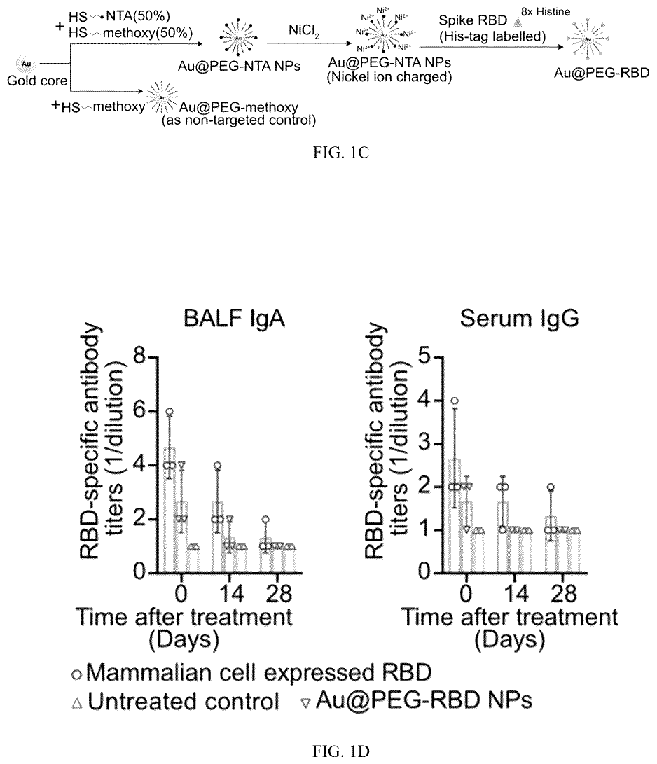

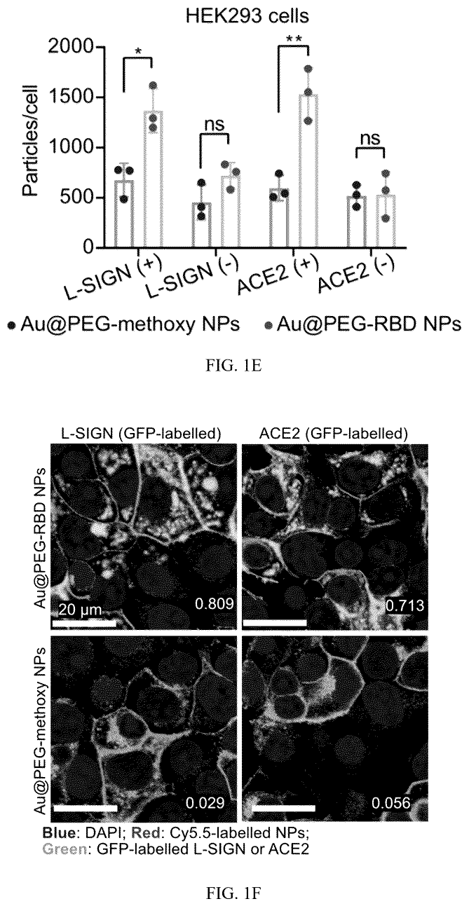

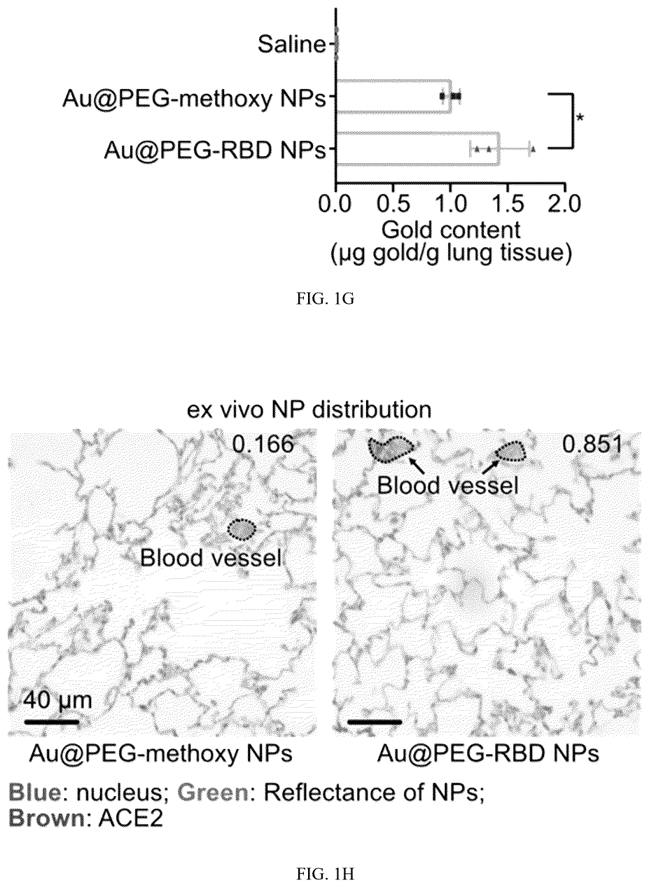

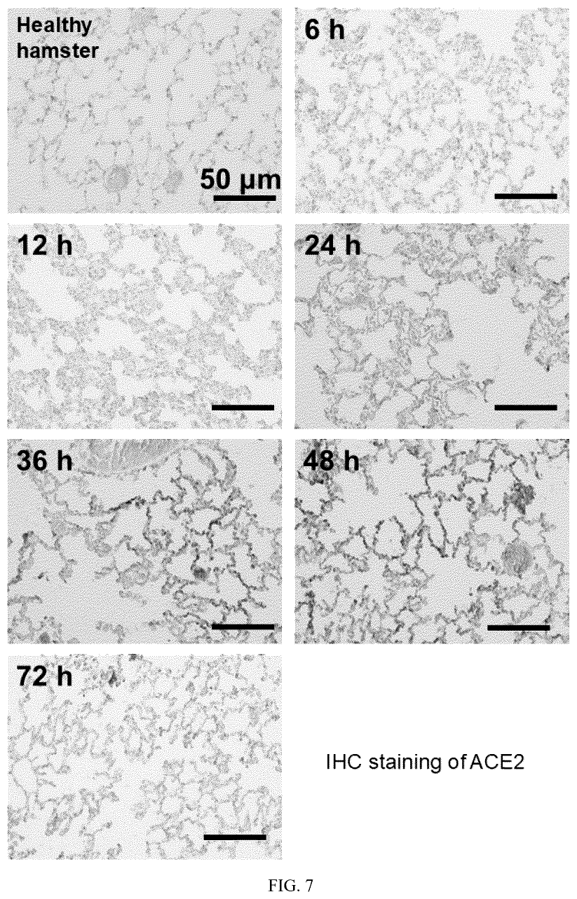

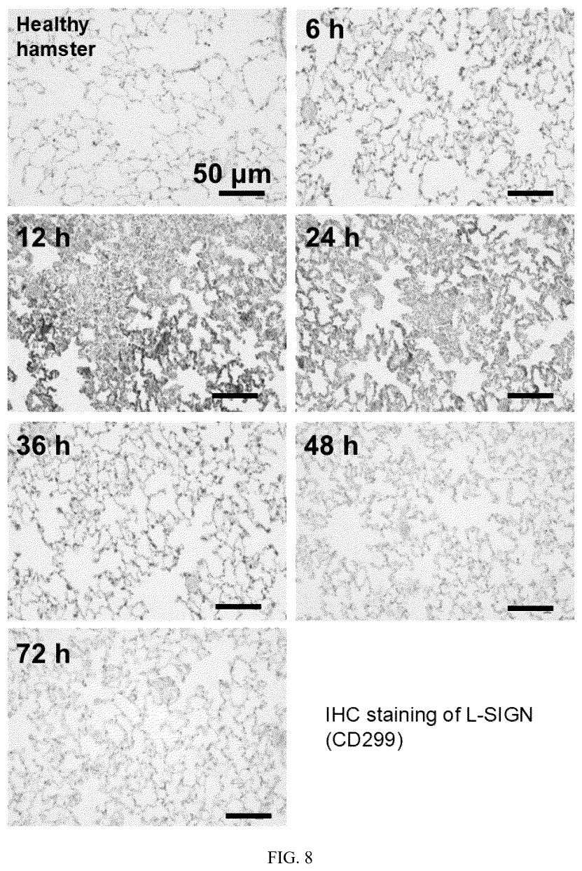

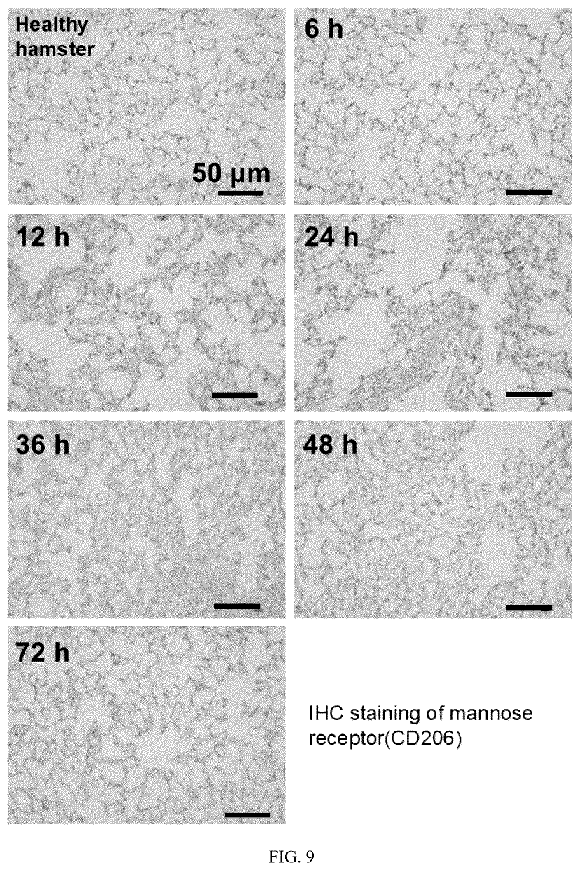

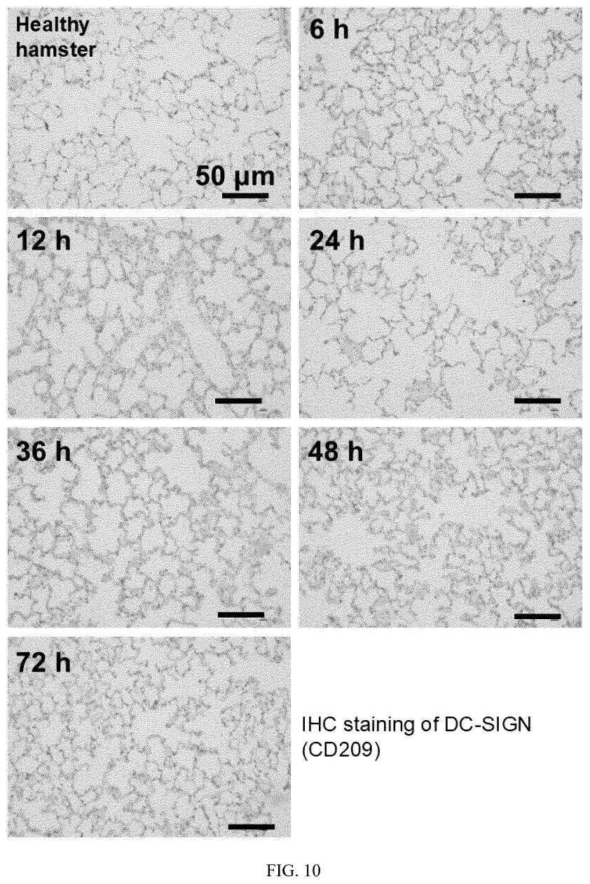

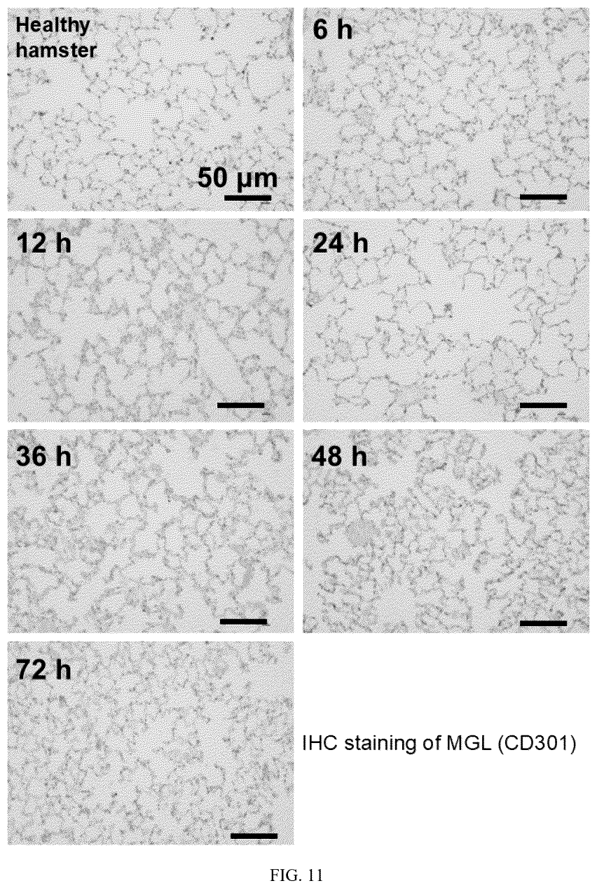

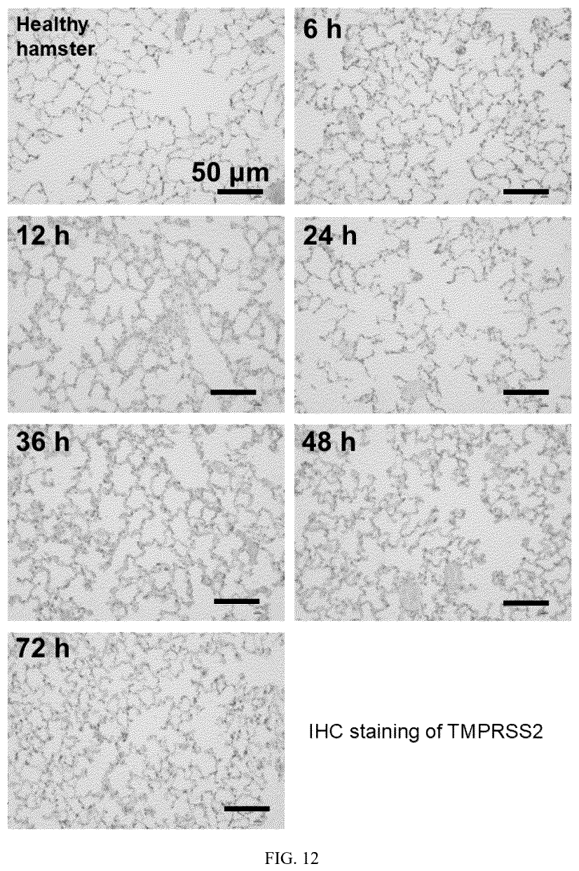

We tracked the expression of six Spike receptors in the lungs of hamsters with LPS-induced ARDS (mimicking bacterial infection, the most common disease cause) by enzyme-linked immunosorbent assay (ELISA) and immunohistochemistry (IHC). Four receptors [dendritic cell-SIGN, mannose receptor, macrophage galactose type C-type lectin (MGL), and transmembrane serine protease 2 (TMPRSS2)] were at healthy, basal levels (see FIGS. 7-12), while two were transiently activated within 48 hours of ARDS induction. L-SIGN expression in alveolar epithelial cells surged 12 hours post-induction and dropped to basal levels 24 hours later, and ACE2 expression grew 36 hours post-induction and dropped to basal levels 12 hours later (see FIGS. 1A and 1B). Such temporally separated activation of both receptors mediates the lung delivery of Au@PEG-RBD NP at different timepoints post ARDS-onset.

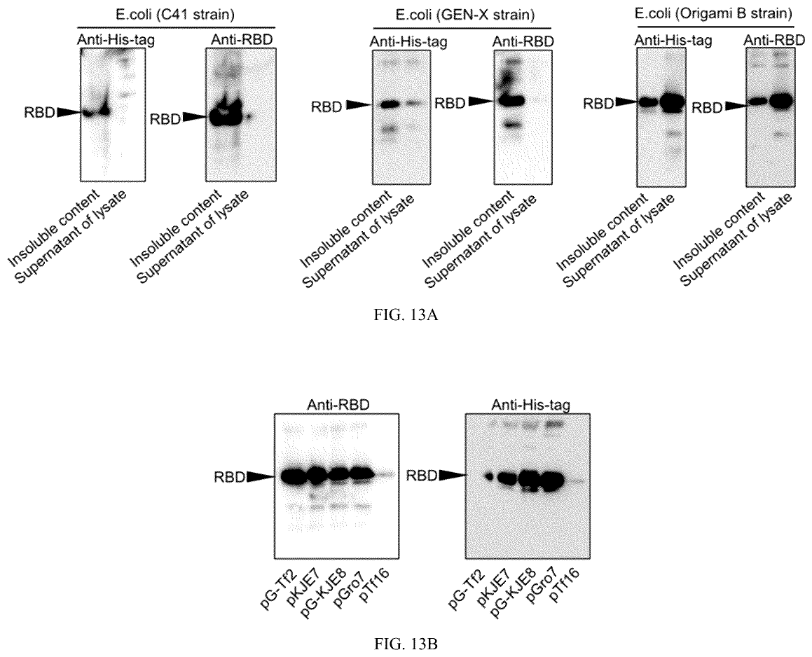

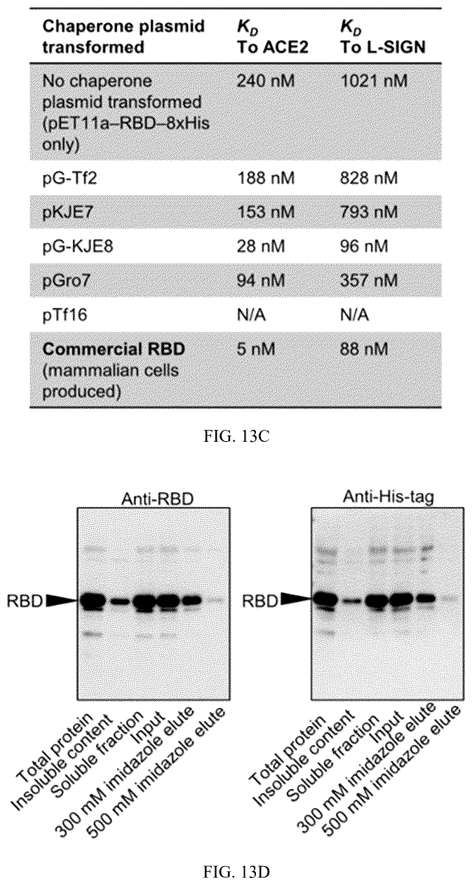

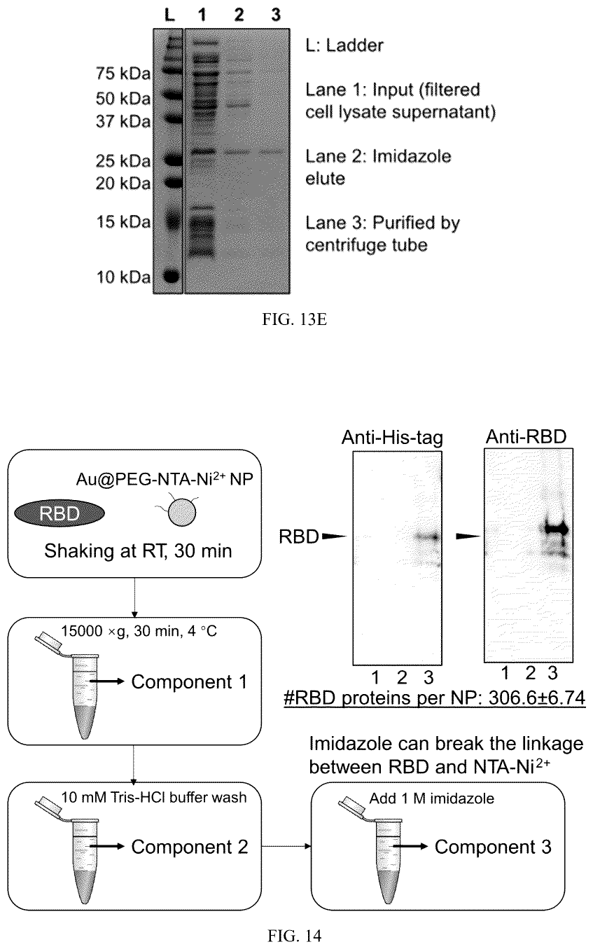

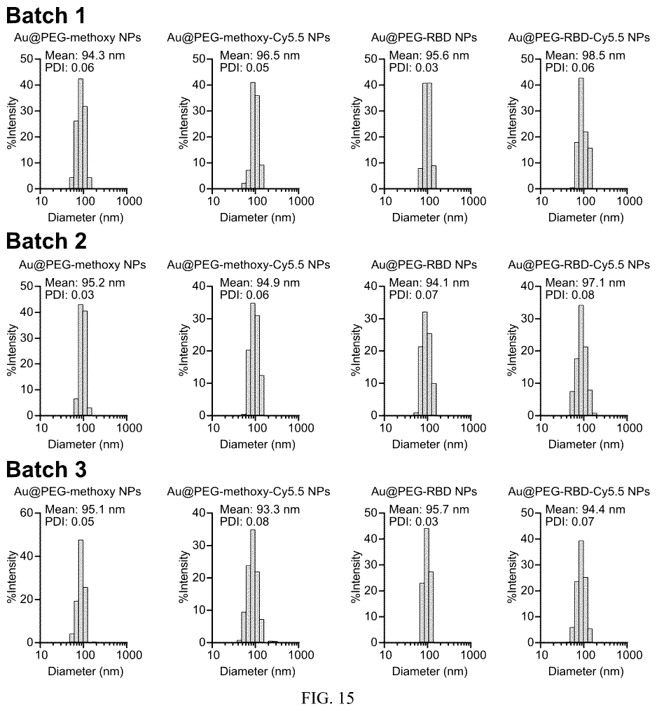

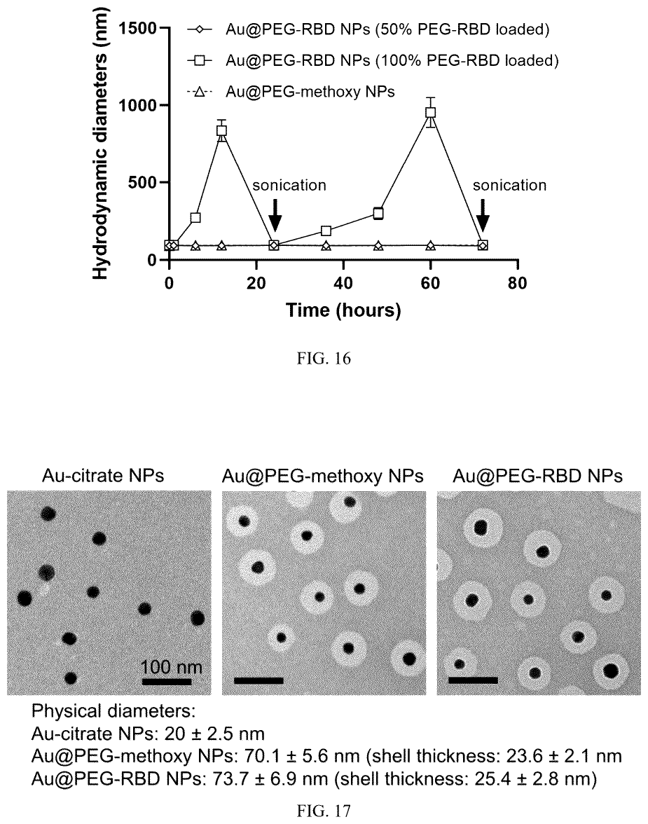

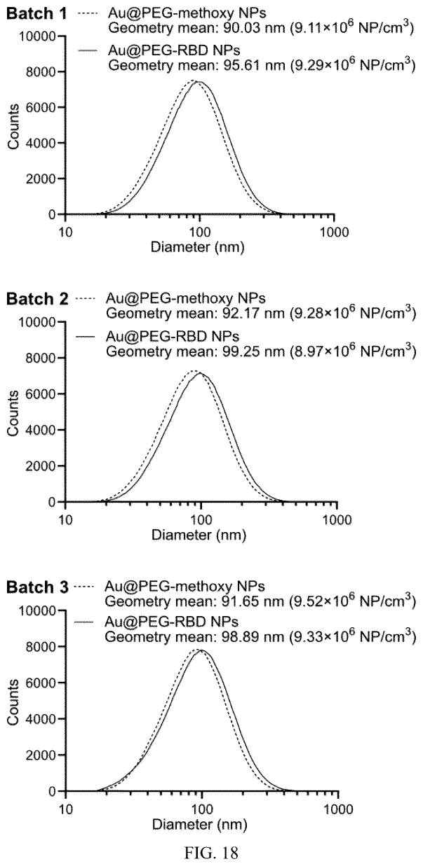

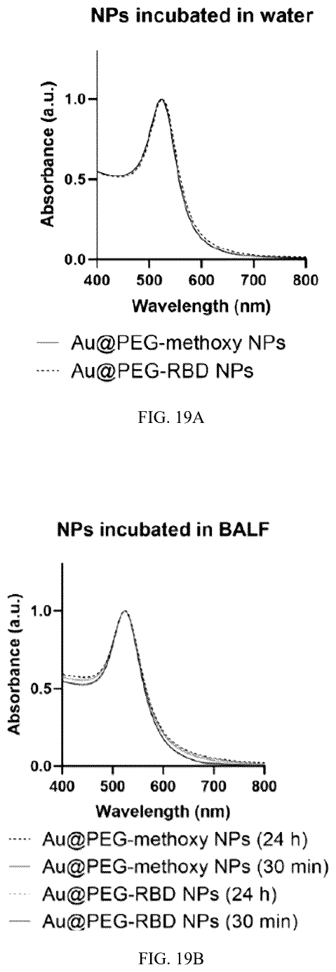

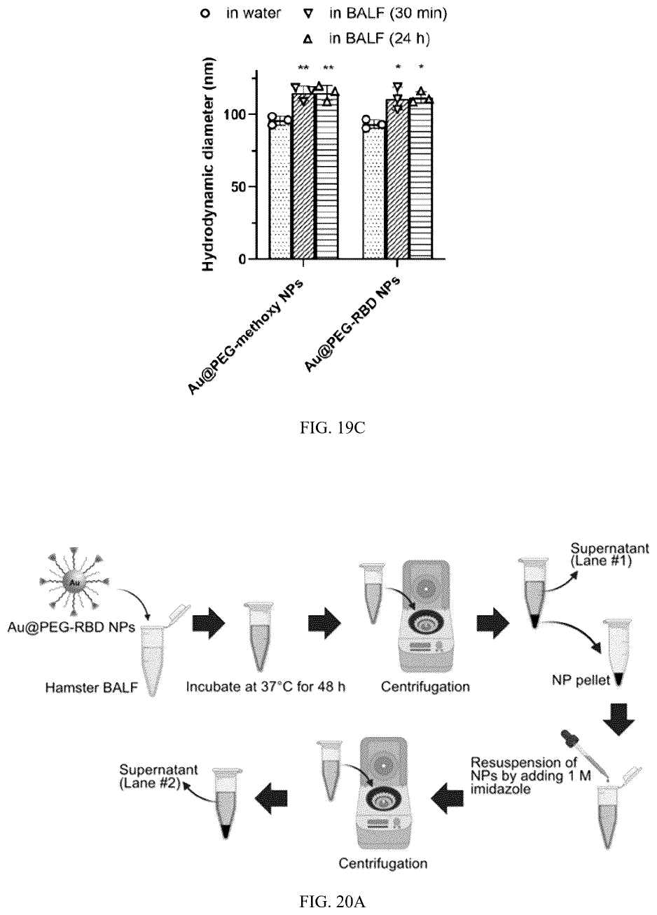

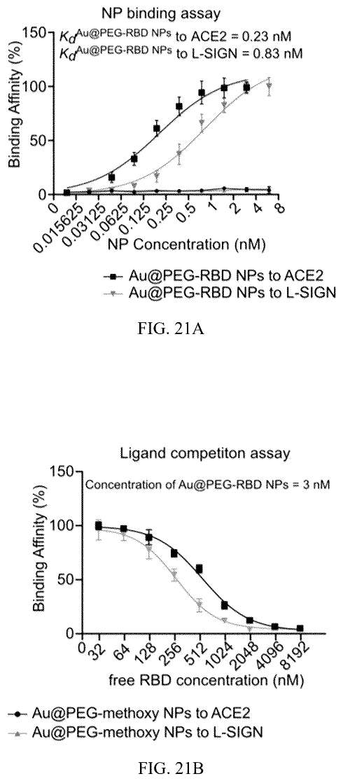

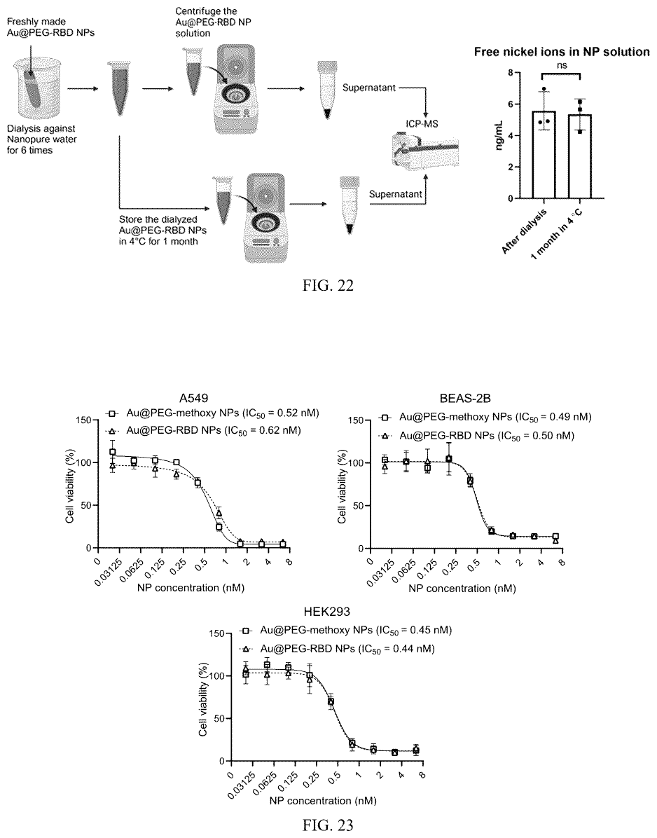

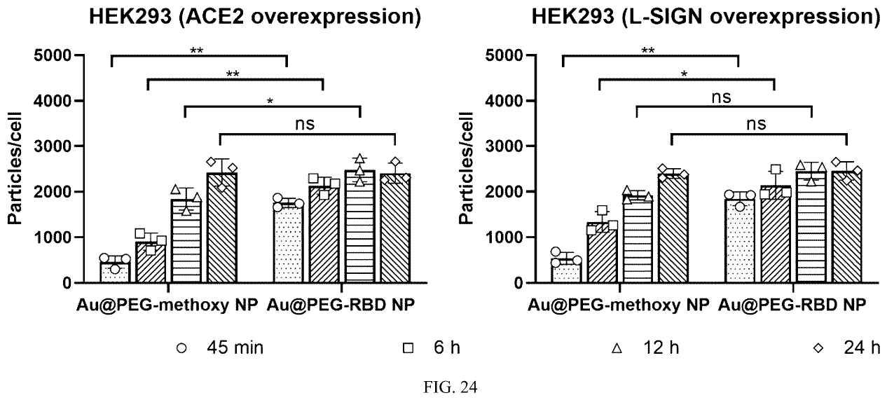

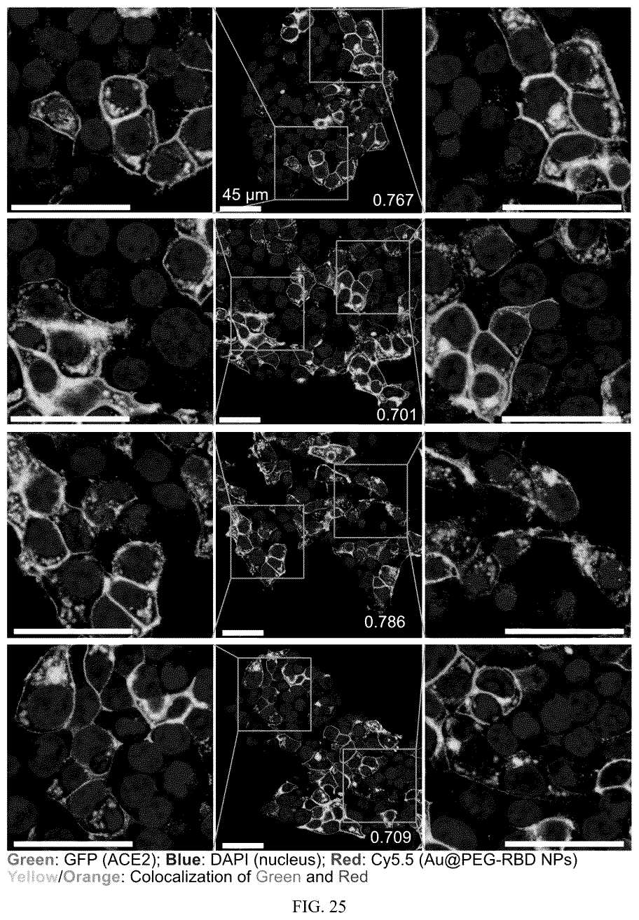

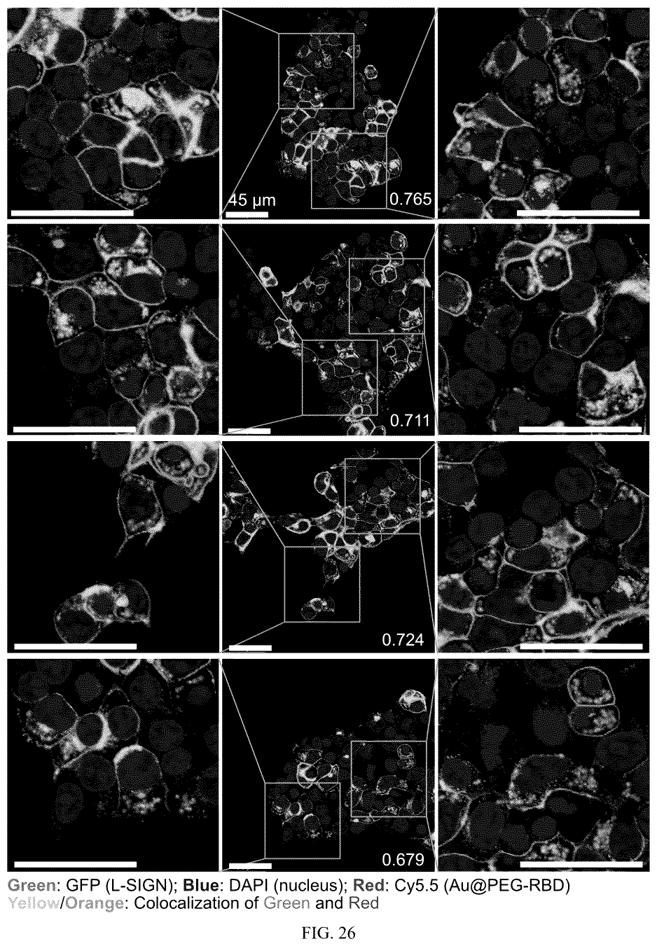

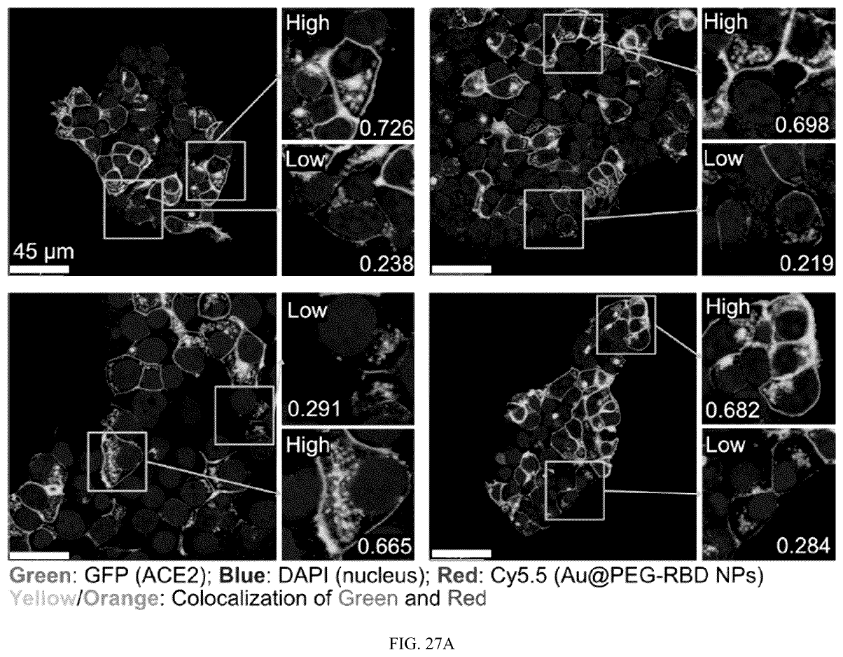

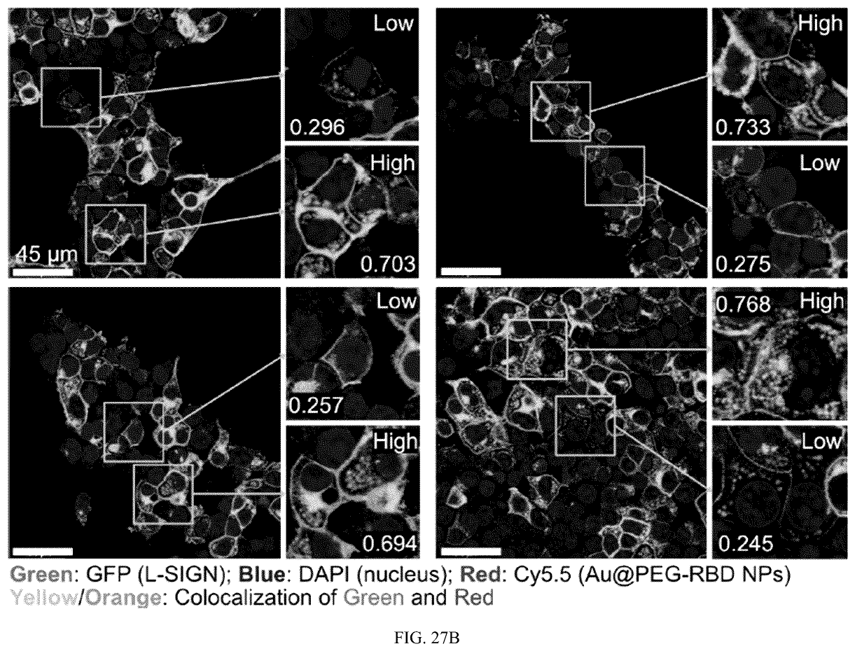

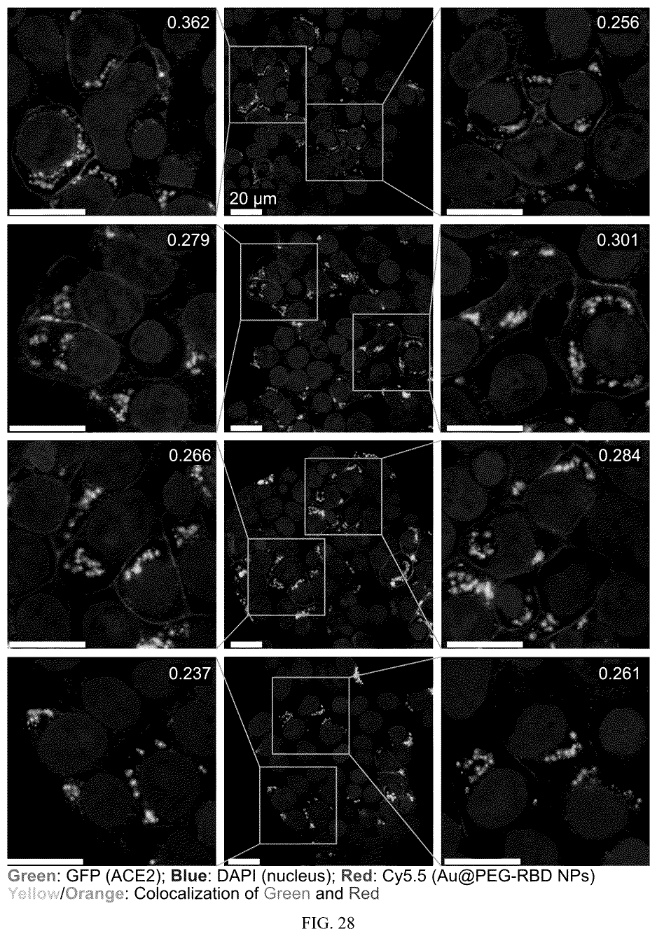

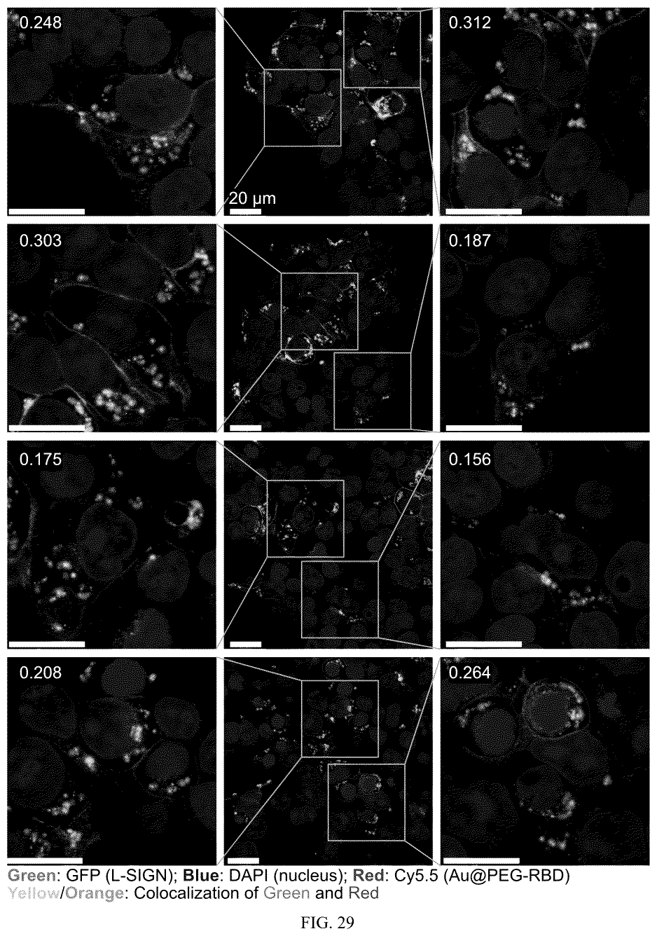

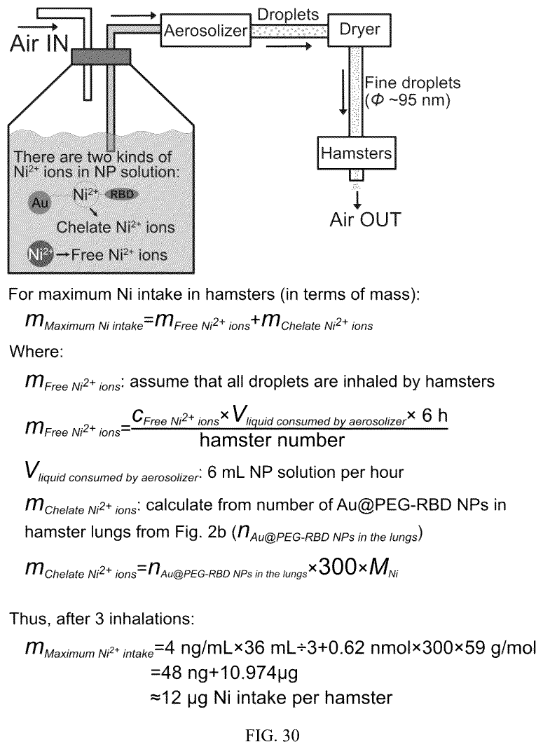

To prepare Au@PEG-RBD NPs, we reacted 20-nm citrate-capped gold cores with a 1:1 molar ratio of about 20 k-Da thiol-PEG-methoxy and thiol-PEG-nitrilotriacetic acid (NTA) via gold-sulfur bonds, followed by attaching recombinant histidine (His8)-tagged RBD proteins using Ni²⁺ ions as chelating agent (see FIG. 1C and FIG. 13). We verified RBD attachment by releasing RBD proteins from the Au@PEG-RBD NP with imidazole (which competes for His8) and blotting them with both antibodies against His and RBD (see FIG. 14). The Au@PEG-RBD NP had a hydrodynamic size of about 94 nm, polydispersity index of about 0.05, and zeta potential of −15 mV as measured by dynamic light scattering; the hydrodynamic size distribution was consistent over multiple batches (see FIG. 15). The NP had about 600 PEG strands and about 300 RBD proteins by Ellman's assay (see Table 5 and FIG. 14). Adding only thiol-PEG-NTA to the gold cores to achieve a full RBD loading caused NP agglomeration, albeit reversible by sonication (see FIG. 16). The TEM imaging of Au@PEG-RBD NP with negative staining revealed a physical size of about 74 nm (comprising a gold core of about 20 nm in size and a PEG shell of about 25 nm thick as shown in FIG. 17). This NP remained stable upon aerosolization [with airborne size (as measured by a scanning mobility particle sizer, SMPS) near-identical to hydrodynamic size (see Table 5 and FIG. 18)] and upon incubation in BALF freshly extracted from healthy Syrian hamsters for 24 hours [mild enlargement to about 120 nm due to BALF adsorption (vide infra for proteomics analysis as shown in Table 6 and FIG. 19)]; western blot and ELISA confirmed no detectable shedding of RBD proteins from the gold NP upon BALF incubation (see FIG. 20). Our in-house NP binding assay revealed that Au@PEG-RBD NP bound to recombinant ACE2 and L-SIGN receptors with dissociation constants (Kd) of 0.23 nM and 0.83 nM by ELISA, respectively, about 100-fold more strongly than free RBD (see FIG. 13C and FIG. 21); such enhanced binding of the NP might stem from the “multivalent effect” whereby NP curvature permits localized surfaces of multiple ligands to interact simultaneously with cell-surface receptors, a result consistent with other receptor-targeting gold NPs. Binding of Au@PEG-RBD NP to ACE2 and L-SIGN receptors under competition by increasing concentrations of free RBD dropped in a dose-dependent manner, additional proof of receptor-specific binding (see FIG. 21). By inductively coupled plasma mass spectrometry (ICP-MS) analysis, repeated rinses of Au@PEG-RBD NP with water and refrigerated storage for 1 month did not cause leaching of free Ni²⁺ ions (see FIG. 22). The NP was not toxic on A549 alveolar epithelial cancer cells, non-cancerous BEAS-2B bronchial epithelial cells, or HEK293 cells (which exhibit epithelial morphology) in vitro (see FIG. 23), nor was it immunogenic to healthy hamsters upon intratracheal instillation. Levels of BALF IgA (marker of lung mucosal immunity) and serum IgG (marker of humoral immunity) remained low up to 28 days post-instillation (see FIG. 1D), as the Au@PEG-RBD NP contains no adjuvant. As untargeted control, we prepared an RBD-free, Au@PEG-methoxy NP with similar physicochemical parameters and stability as Au@PEG-RBD NP but without binding to L-SIGN and ACE2 (see Tables 5 and 6, and FIG. 21A).

We validated the receptor binding of both NP types to ACE2 and L-SIGN in vitro and ex vivo. ICP-MS analysis revealed that Au@PEG-RBD NP associated with HEK293 cells that overexpress ACE2 and L-SIGN more abundantly than naïve HEK293 cells, yet Au@PEG-methoxy NP showed low association to all cell types 1 h post-incubation in vitro (see FIG. 1E). Such preferential cellular association only took place over shorter incubation periods, up to 12 hours; over longer incubation periods (24 hours), Au@PEG-RBD NP associated with HEK 293 cells at similar amounts independent of L-SIGN or ACE2 expression level (see FIG. 24). Confocal immunofluorescence images verified the preferential entry of Cyanine 5.5-labeled Au@PEG-RBD NP to HEK293 cells overexpressing ACE2 and L-SIGN [both fused with green fluorescent protein (GFP)] 1 hour post-incubation (see FIG. 1F). The Pearson colocalization coefficient (PCC) between both receptors and Au@PEG-RBD NPs was 0.7-0.8, indicating strong overlap. HEK293 cells with low receptor expression levels also revealed intracellular localization of Au@PEG-RBD NP, albeit less abundantly and with weaker overlap with receptors (PCC: 0.3-0.4 as shown in FIGS. 25-29). Not all cells expressed ACE2 or L-SIGN given the modest efficiency of the commercial transfection reagent used. Collectively, these results bolster our claim of the preferential uptake of Au@PEG-RBD NP by L-SIGN/ACE2-expressing cells. When intratracheally instilled to healthy hamsters, ICP-MS analysis of the extracted lungs revealed 48% higher association of Au@PEG-RBD NP than Au@PEG-methoxy NP ex vivo (see FIG. 1G), a reasonably mild increase because ACE2 is only expressed in a low population of epithelial cells (mainly type II; about 10% of the whole epithelium). Confocal reflectance imaging, coupled with silver-enhanced gold NPs (which can reflect light) and IHC, depicted the preferential entry of Au@PEG-RBD NP to ACE2-expressing alveolar cells and the low entry of Au@PEG-methoxy NP to all cell types, independent of ACE2 expression (see FIG. 1H). We did not study NP colocalization with L-SIGN given its low expression in healthy hamsters.

### EXAMPLE 2—INTRAPULMONARY DISTRIBUTION OF INHALED Au@PEG-RBD NPs

In a typical inhalation experiment, our dose regimen was three inhalations of Au@PEG-RBD NP for 2 hours each with the following justifications: ARDS hamsters inhaled aerosolized NPs for 2 hours and were then sacrificed immediately or returned to the holding cage for 10 hours. This iteration lasted till the third inhalation, the earliest timepoint for Au@PEG-RBD NP to reduce the pro-inflammatory cytokines to baseline level (see FIG. 2A). A shorter inhalation time (for example, 20 minutes) would reduce animal distress as no food or water should be provided during inhalation, but it requires a higher NP inhalation concentration for attaining the same therapeutic efficacy that would block the aerosolizer. Our inhalation dose was 9-10 million NPs per mL of inhaled compressed air for 150-g hamsters (as measured by SMPS as shown in Table 5), a reasonable equivalent dose per FDA guidelines given^{[24]} our past result that ˜3 million NPs per mL was a safe concentration for inhalation by 25-g mice^{[25]}. The cumulative nickel intake by hamsters over three inhalations was below the safety limit, based on the FDA equivalent dose for humans (see FIG. 30).

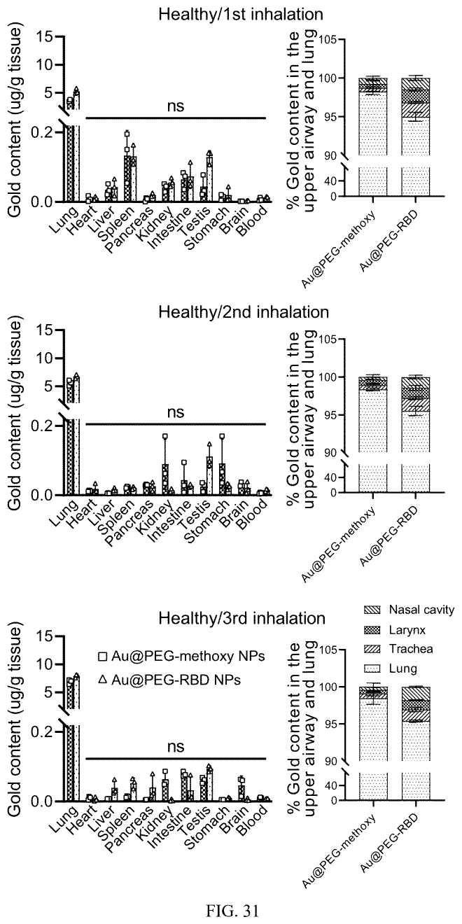

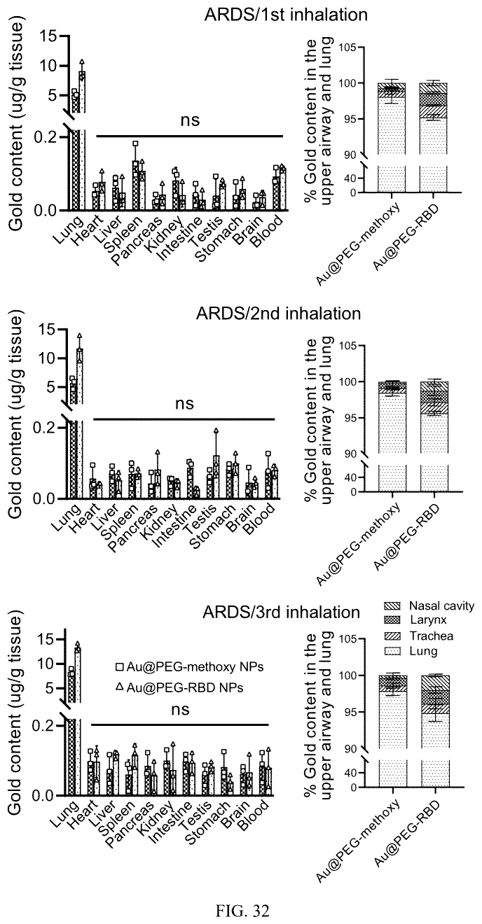

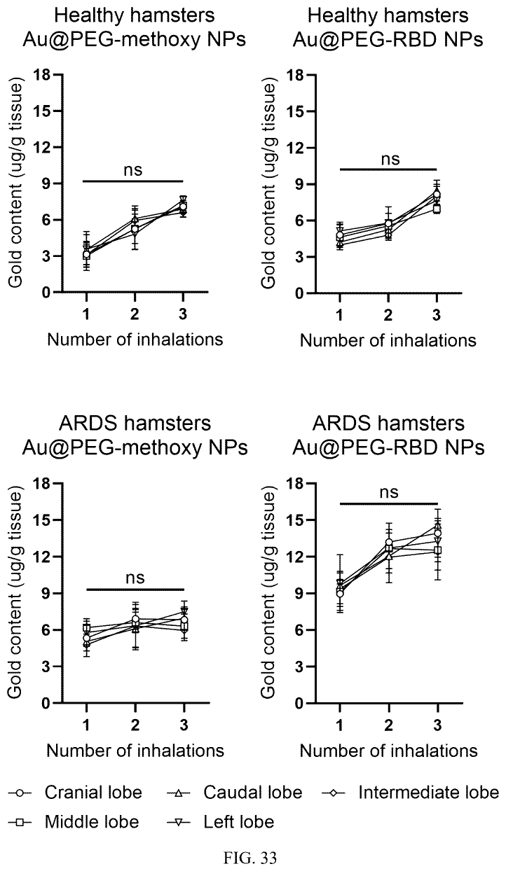

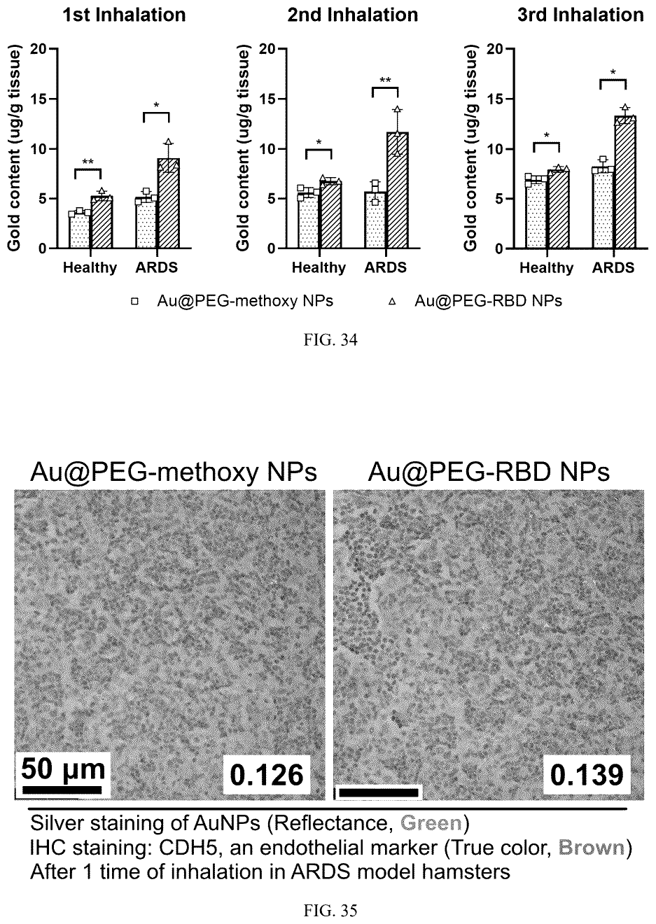

In healthy and LPS-induced ARDS hamsters, both NP types accumulated in the lower airway (lung) rather than the upper airway (nasal cavity, larynx, and trachea), were evenly distributed among the five lung lobes, and were undetectable in major organs (see FIGS. 31-33). Both NP types deposited in the lungs of healthy hamsters similarly, but Au@PEG-RBD NP deposited in ARDS lungs more abundantly than Au@PEG-methoxy NP. Accumulation of Au@PEG-RBD NP was higher in ARDS lungs than healthy lungs, proof of disease-selective delivery (see FIG. 2B and FIG. 34). On tissue-level distribution, 60-80% of both NP types resided in the lavaged lung (obtained by extracting BALF from the whole lung) and the remaining in BALF (containing infiltrated immune cells and supernatant). Also, 20-30% of both NP types diffused from BALF supernatant to the lavaged lung tissue in healthy hamsters, and 10% diffused from BALF supernatant to the infiltrated BALF cells in ARDS hamsters (see FIG. 2C). Confocal reflectance imaging with IHC captured more abundant deposition of Au@PEG-RBD than Au@PEG-methoxy NPs in the ARDS lung, and stronger association of Au@PEG-RBD NP with epithelial cells [Pearson correlation coefficient (PCC): 0.824 vs. 0.183]; both NP types barely associated with the infiltrated endothelial cells (PCC ˜0.1-0.2) (see FIG. 2D and FIG. 35). There was a stronger overlap of Au@PEG-RBD NP with L-SIGN (PCC: 0.889 vs. 0.336) and ACE2 (PCC: 0.793 vs. 0.281) than Au@PEG-methoxy NP in the ARDS lung (see FIG. 2E).

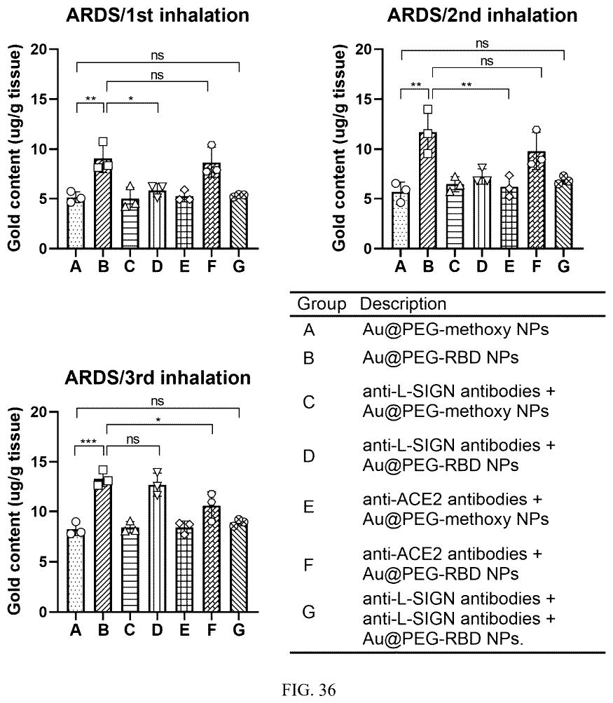

To verify the specific binding of Au@PEG-RBD NP to the lungs via L-SIGN or ACE2 in vivo, we intranasally injected antibodies against ACE2 or L-SIGN into LPS-induced ARDS hamsters before each round of NP inhalation. We denote “100%” as the lung gold content in the case of “full receptor targeting” achievable by Au@PEG-RBD NP without any antibody blocking and “0%” in the case of “no receptor targeting” achievable by Au@PEG-methoxy NP. We detected drastically reduced accumulation of Au@PEG-RBD NP in the ARDS lungs due to L-SIGN blocking, where the accumulation of Au@PEG-RBD NP was similar to that of Au@PEG-methoxy NP with antibody blocking upon the first and second inhalations (12 hours and 24 hours post-LPS induction); yet, such reduction was not evident upon the third inhalation when L-SIGN was no longer activated by LPS (36 hours post-LPS induction). These data match the initial activation of L-SIGN and basal level of ACE2 (see FIG. 1A). Conversely, when we blocked ACE2, reduction of NP deposition in the lungs occurred upon the second and third inhalations, not the first inhalation (see FIG. 2F and FIG. 36). These results corroborate the eventual basal level of L-SIGN and activation of ACE2 (see FIG. 1A). Collectively, L-SIGN predominantly contributed to lung delivery of Au@PEG-RBD NP at the initial phase post-ARDS onset while ACE2 at the late phase.

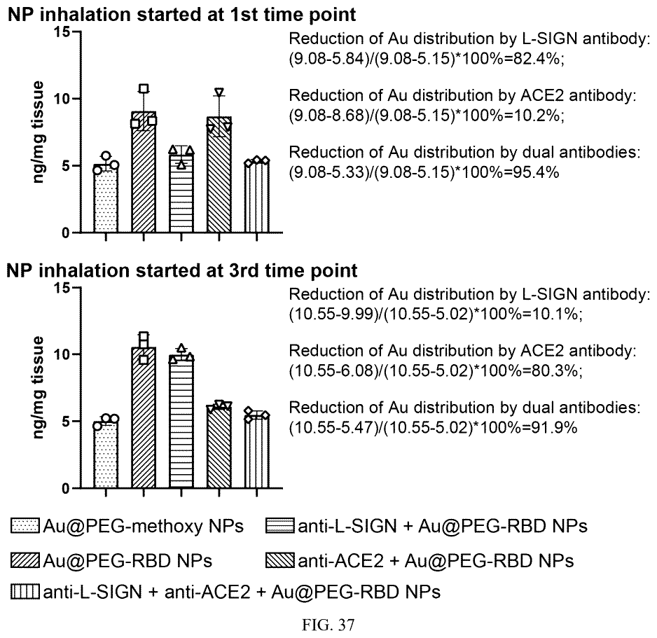

Because L-SIGN and ACE2 were activated at distinct peak timepoints post-ARDS onset, they were unlikely to mediate the lung delivery of Au@PEG-RBD NP cooperatively. To probe the relationship between both receptors, we studied how antibody blocking of L-SIGN, ACE2, or both receptors affected the lung delivery of one single dose of Au@PEG-RBD NP inhaled at different timepoints post-ARDS onset. At the first timepoint (12 hours post-LPS induction when L-SIGN expression peaked and exceeded ACE2), single blocking of L-SIGN and ACE2 reduced the lung accumulation of Au@PEG-RBD NP by 83% and 10%, respectively, and blocking both receptors 95%. Conversely, at the third timepoint (36 hours post-LPS induction when ACE2 expression peaked and exceeded L-SIGN), single blocking of L-SIGN and ACE2 reduced lung accumulation by 10% and 80%, respectively, and blocking both receptors 92%. Therefore, both receptors mediated lung delivery additively (see FIG. 37).

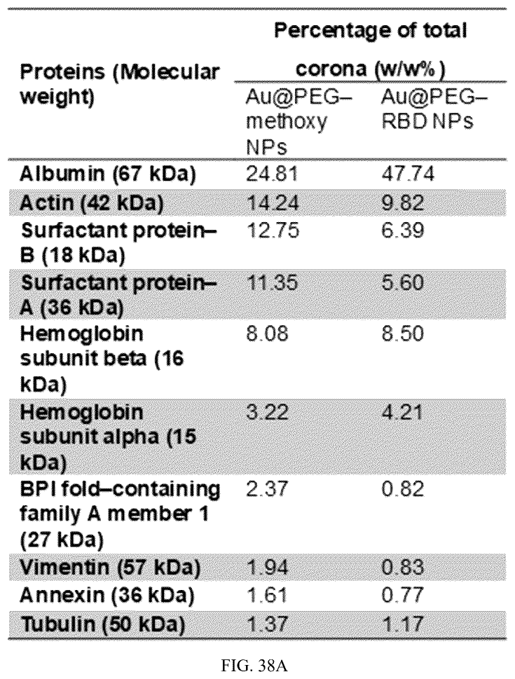

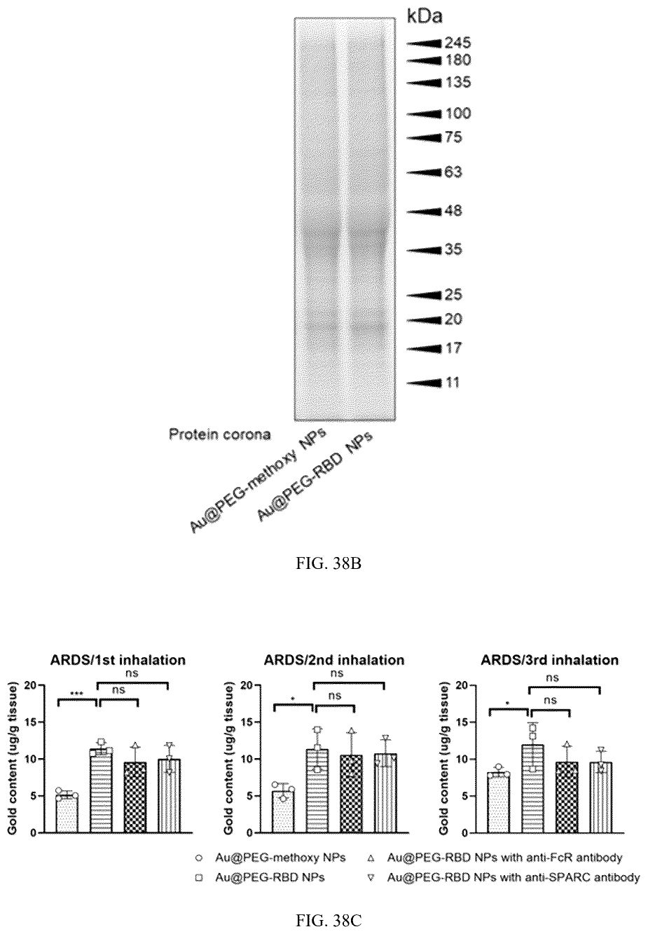

Further, we verified that BALF protein corona did not affect receptor targeting. For both NP types, the most abundant corona proteins were albumin, actin, lung surfactant proteins, and hemoglobin subunits (>70% mass ratio) by liquid chromatography tandem mass spectrometry (LC-MS/MS), and the amounts of proteins bound were similar; the key difference lies in the higher association of albumin to Au@PEG-RBD than Au@PEG-methoxy NPs (47.74 vs. 24.81%) (see FIG. 38A). Lastly, we tested if blocking albumin-related receptors affected the lung accumulation of NPs. Intranasal injection of antibodies against Fc or Secreted Protein Acidic and Rich in Cysteine (SPARC), both receptors of albumin [26], to ARDS hamsters did not severely attenuate the lung accumulation of Au@PEG-RBD NP (see FIG. 38C).

### Efficacy of Au@PEG-RBD NPs in LPS-Induced ARDS Hamsters

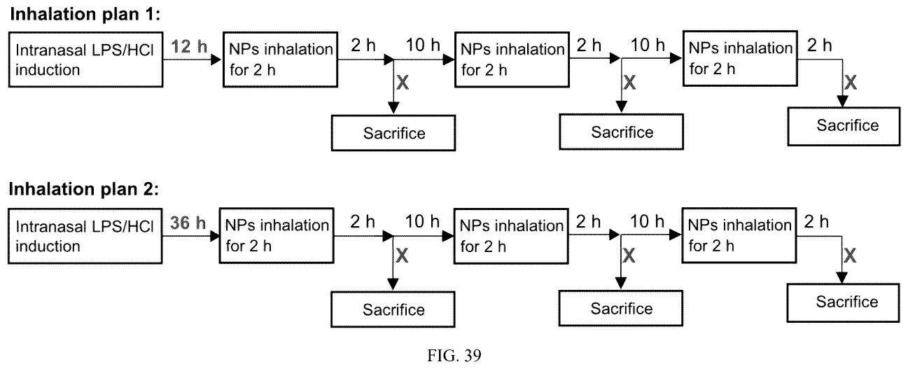

For efficacy studies, we followed the same dose regimen as justified in the previous section on biodistribution studies: three inhalations of 2 hours each for Au@PEG-RBD NP (see FIG. 2A and FIG. 39). The treatment groups were Au@PEG-RBD NP, Au@PEG-methoxy NP (same gold dose as Au@PEG-RBD NP), hydrocortisone (intraperitoneal, 5 mg-drug/kg-hamster/12-h, 3 times) as a positive control drug, and untreated (n=9).

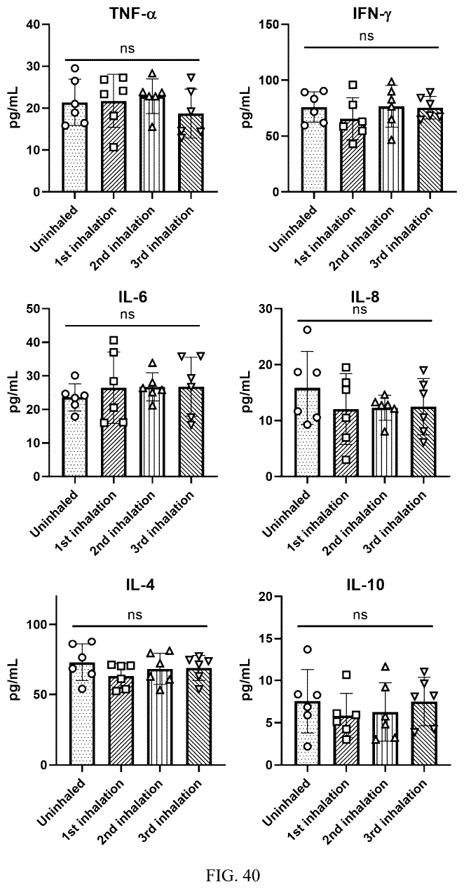

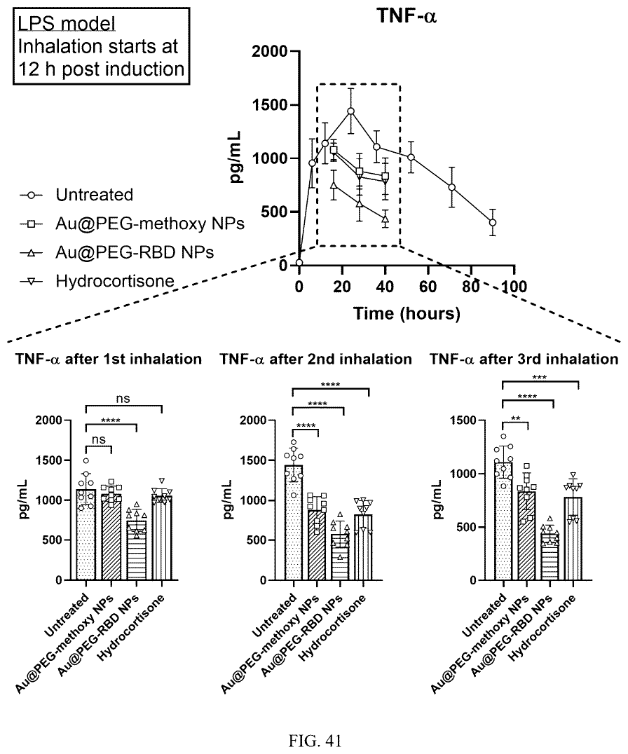

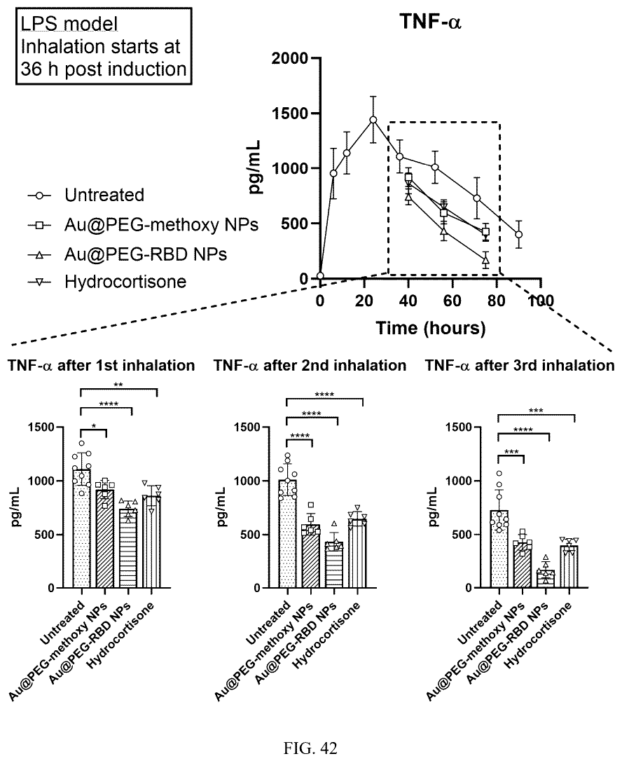

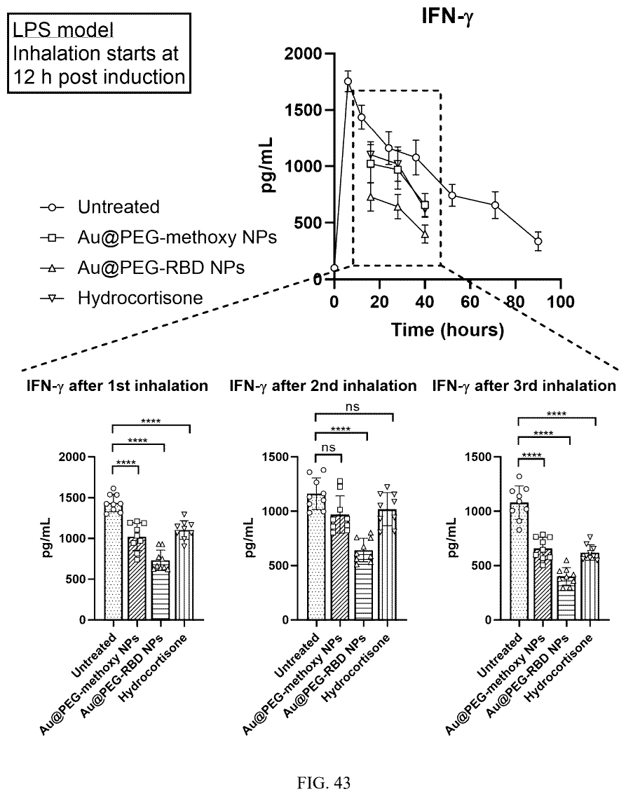

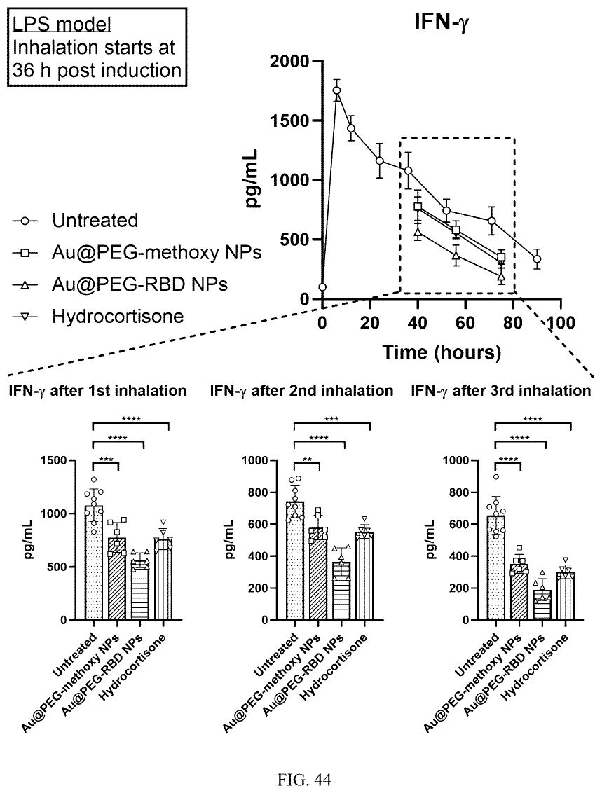

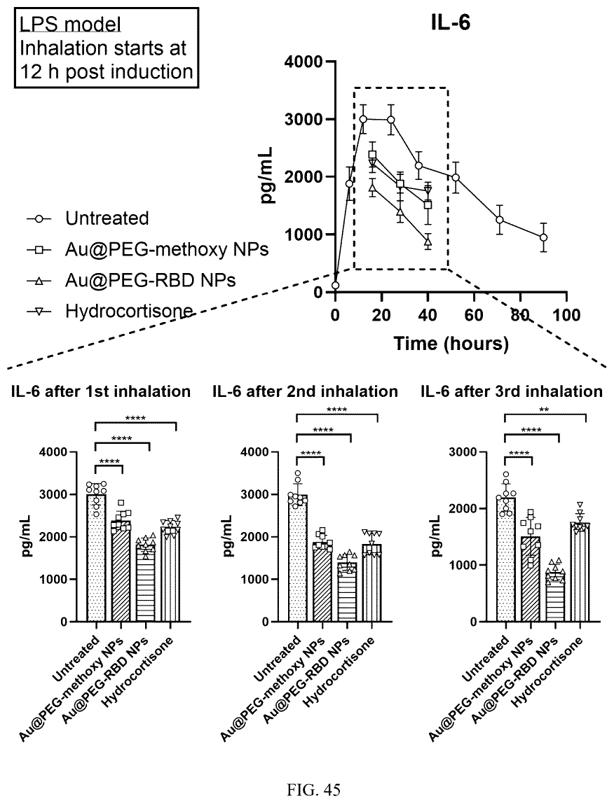

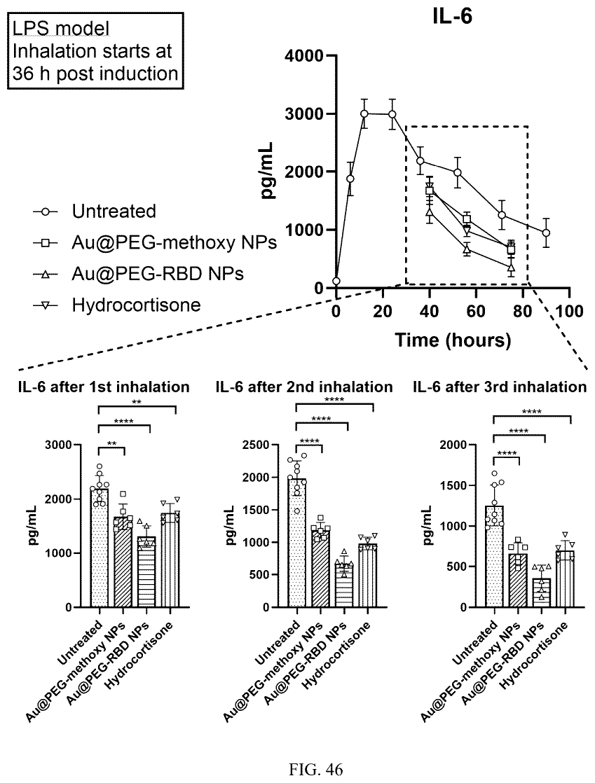

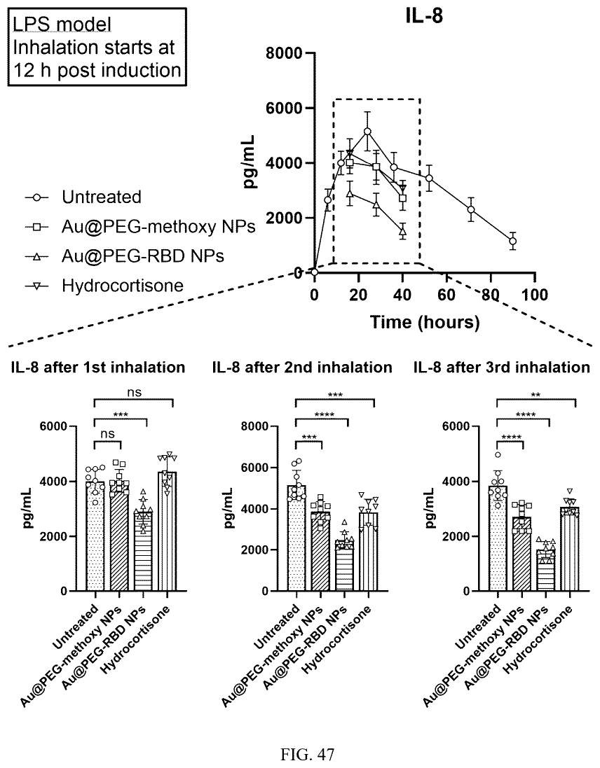

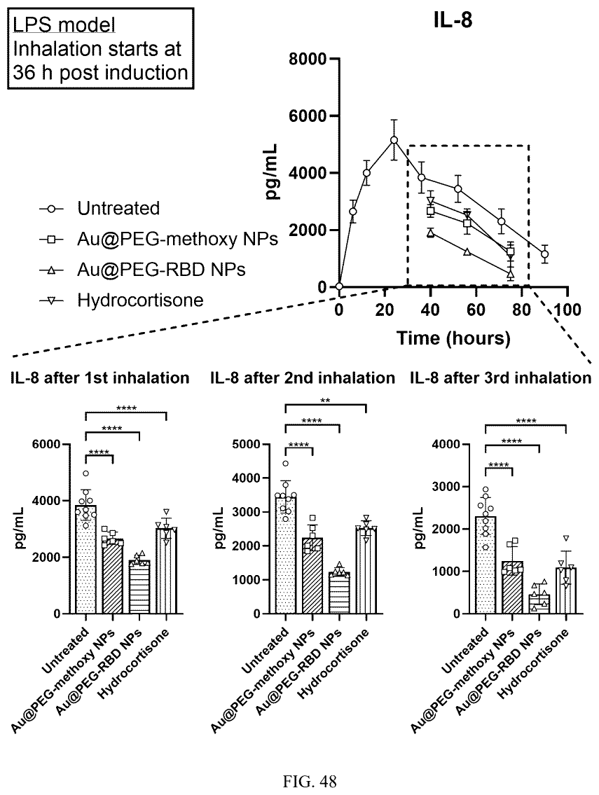

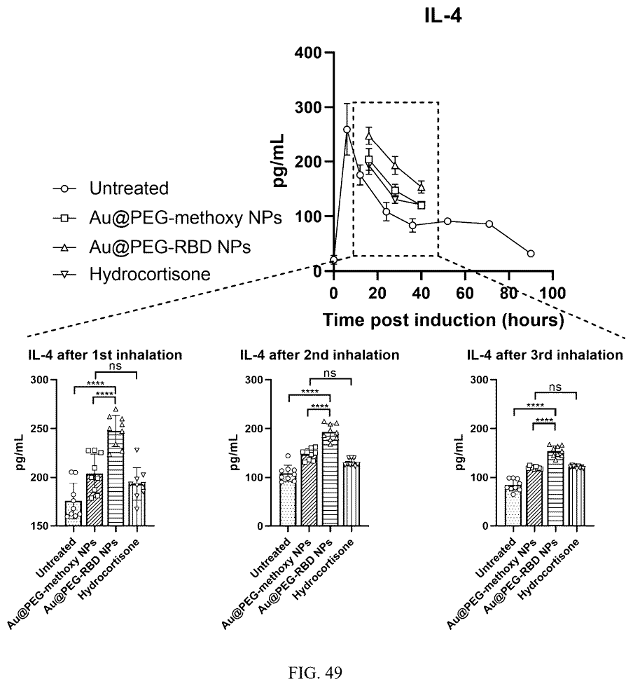

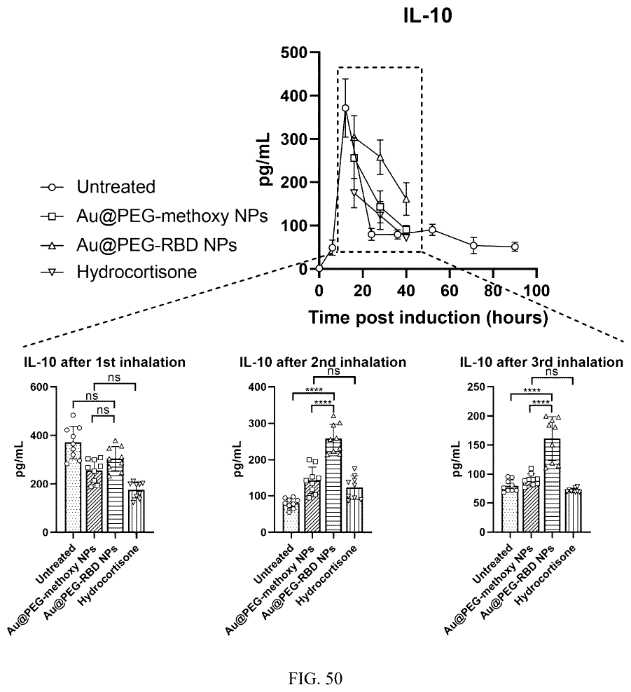

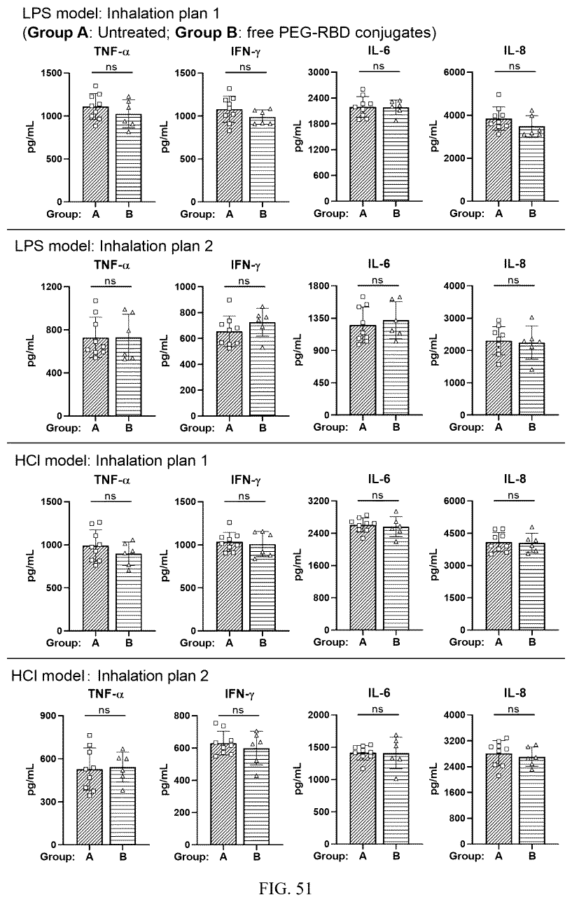

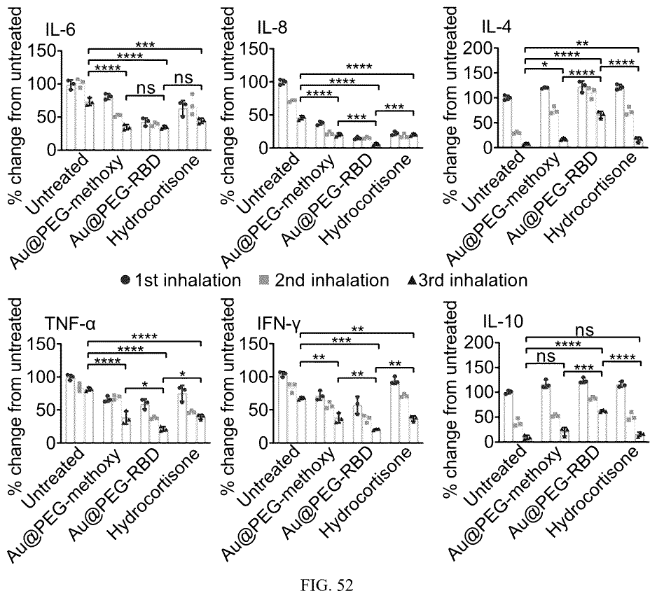

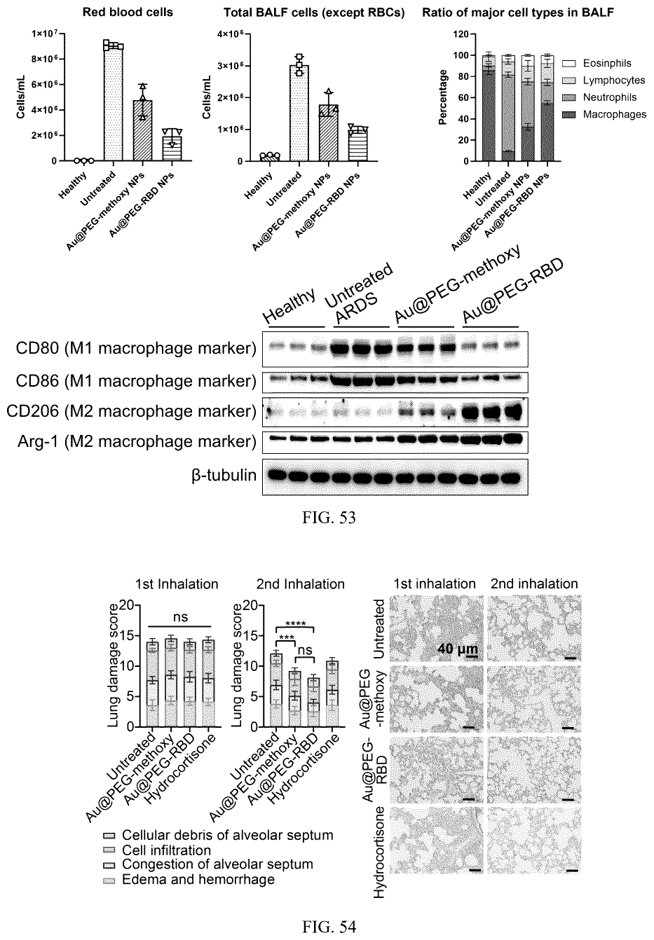

In untreated LPS-induced ARDS hamsters, the levels of proinflammatory cytokines (TNF-α, IFN-γ, IL-6, and IL-8) in BALF supernatant peaked 12-24 h post-induction, but they returned to basal levels in healthy hamsters 96 hours post-induction (FIG. 3A and FIG. 40). Of all groups tested, Au@PEG-RBD NP most rapidly and severely reduced the four cytokines in BALF after only one inhalation; Au@PEG-methoxy NP and hydrocortisone also inhibited the same markers but required 2-3 administrations (see FIG. 3A and FIGS. 41-48). Moreover, Au@PEG-RBD NP increased the levels of anti-inflammatory cytokines IL-4 and IL-10 (both contributors to tissue repair in the lungs) in BALF cells in the most pronounced and sustained manner over three inhalations (see FIG. 3A and FIGS. 49 and 50). As the non-therapeutic, negative control, free RBD-PEG conjugate without gold (72 μmol-RBD/kg-hamster; same RBD dose as Au@PEG-RBD NP) was not effective (see FIG. 51), excluding PEG or RBD as the source of efficacy and hence enabling the attribution of efficacy specifically to Au@PEG-RBD NP. Accordingly, we reasoned that Au@PEG-RBD NP was more effective than Au@PEG-methoxy NP due to its more abundant cellular delivery of gold cores. Au@PEG-RBD NP did not alter BALF cytokine levels in healthy hamsters, evidence of ARDS-specific treatment (see FIG. 40). A similar efficacy trend was detected based on the levels of four pro-inflammatory cytokines and two anti-inflammatory cytokines in the lavaged lung tissue by western blot (see FIG. 3B and FIG. 52) and intracellular reactive oxygen species (ROS) of BALF cells (see FIG. 3C). By counting the extracted BALF cells with Wright-Giemsa staining, we showed that Au@PEG-RBD NP reduced the counts of red blood cells (indicator of hemorrhage), total white blood cells (indicator of immune cell recruitment^{[27]}), and neutrophils (most abundant cell type in BALF whose activation and infiltration to the lung for promoting tissue injury is a hallmark of ARDS^{[28]}) (see FIG. 53). Further, western blot analysis of BALF cells showed that Au@PEG-RBD NP downregulated markers of M1 macrophage (CD80 and CD86) yet upregulated markers of M2 macrophage (CD206 and arginase-1), evidence of repolarization of BALF macrophages from the pro-inflammatory to anti-inflammatory phenotype. Collectively, these results suggested that the reduction in ROS levels in BALF cells was primarily mediated by immune cells.

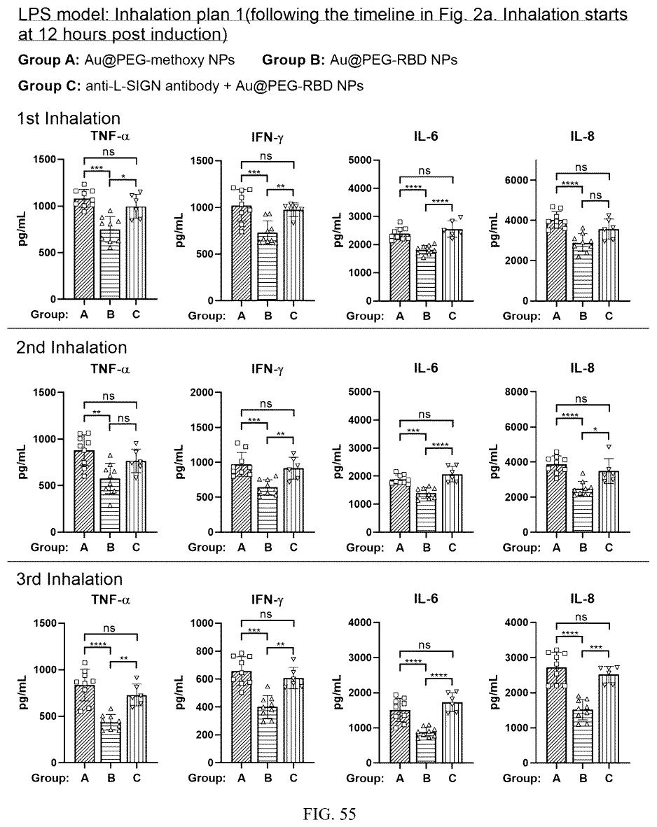

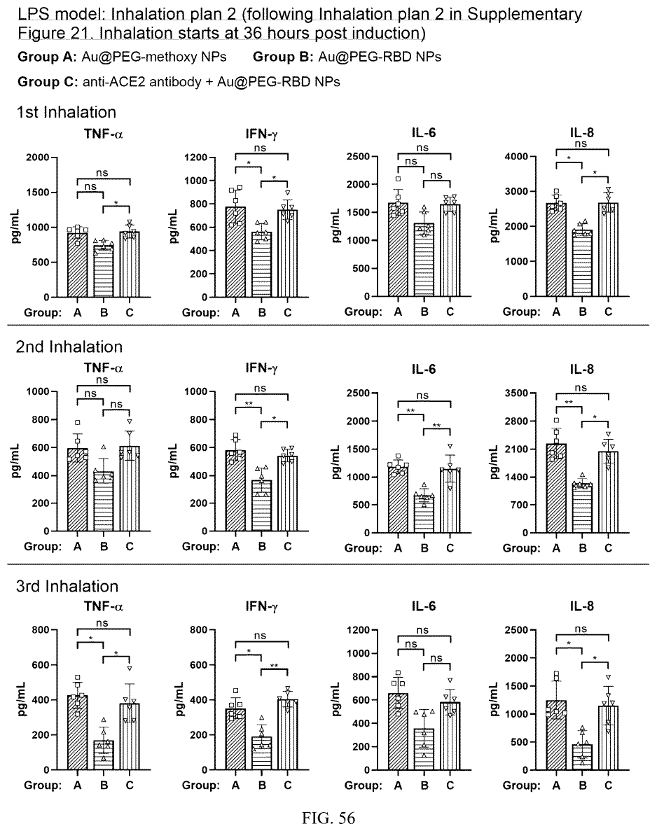

Next, we histologically examined the central and lateral parts of the ARDS lung. Contrary to healthy lungs, ARDS lungs lack net-like structures due to infiltration of immune cells and red blood cells that cause edema and congestion. The first inhalation did not reduce tissue injury in all groups, but after three inhalations, both NP types restored the net-like tissue structures and eliminated hemorrhage, and there were fewer immune cells in the Au@PEG-RBD than Au@PEG-methoxy groups; pronounced blood cell infiltration persisted in the lungs of the untreated and hydrocortisone groups, indicating ABB leakage. Scoring of the degree of lung tissue injury^{[29]} revealed that Au@PEG-RBD NP was most effective, yielding efficacy after two inhalations. (see FIG. 3D, FIG. 54, and Table 7). Tissue restoration was slower than inhibition of cytokines and ROS as it takes time to resolve inflammation and regenerate epithelial cells. In another competition experiment, we intranasally injected antibodies against L-SIGN before Au@PEG-RBD NP inhalation and detected its declined efficacy to the level of Au@PEG-methoxy NP (see FIG. 3E and FIGS. 55, 56, and 72), suggesting that Au@PEG-RBD NP yielded more rapid and potent efficacy than Au@PEG-methoxy NP by targeting L-SIGN activated during ARDS.

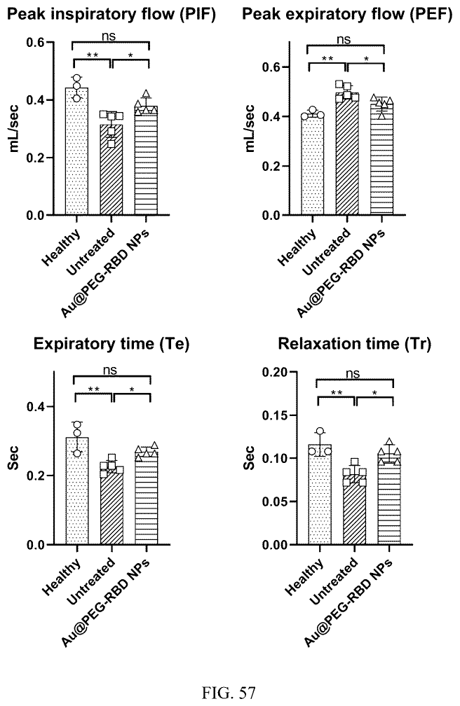

We verified the efficacy of Au@PEG-RBD NP using whole body plethysmography (WBP), a non-invasive tool for measuring respiratory function in unconstrained animals. On hamsters, recent reports used WBP to study disease mechanism^{[30]} and drug response of COVID-19-linked lung injury, but its application for non-viral ARDS remained scarce. In LPS-induced ARDS mice, impaired lung function due to the inflammatory response and vascular leakage led to a stiffer lung tissue, reduced compliance, and increased effort for breathing;^{[31]} in hamsters, we similarly showed that LPS induction led to erratic breathing patterns, a higher breathing rate, a lower tidal volume, and a lower peak inspiratory flow. Moreover, LPS induction enhanced peak expiratory flow and reduced relaxation time, matching past data on COVID-19-induced ARDS mice^{[32]}. Further, LPS induction increased “enhanced pause” (Penh), an indicator of airway resistance^{[31]}. Notably, three inhalations of Au@PEG-RBD NP restored these lung function parameters to healthy levels (see FIG. 3F and FIG. 57). While scientifically instructive, the translational relevance of this WBP study has two limitations. ARDS patients cannot perform lung function tests as they are sedated and intubated, receiving non-invasive ventilation. Additionally, lung function tests are relevant only after patients survive the acute stage.

The untargeted NP, while initially slower to deposit in the lungs, later reached the same lung concentration as the targeted NP as shown in FIG. 34. Then, antibodies were intranasally injected against L-SIGN before Au@PEG-RBD NP inhalation and detected its declined efficacy to the level of Au@PEG-methoxy NP as shown in FIG. 3E and FIG. 6E. This result suggests that Au@PEG-RBD NP yielded more rapid and potent efficacy than Au@PEG-methoxy NP by targeting the upregulated L-SIGN during ARDS. Free RBD-PEG conjugate without gold (72 μmol-RBD/kg-hamster; same RBD dose as Au@PEG-RBD NP) did not reduce ARDS as shown in FIG. 51, enabling us to exclude PEG or RBD as the source of efficacy. Overall, it reveals that the anti-ARDS efficacy stems from the gold core and that RBD is merely a targeting ligand.

### EXAMPLE 3—MECHANISM FOR ANTI-ARDS EFFICACY OF GOLD NPS

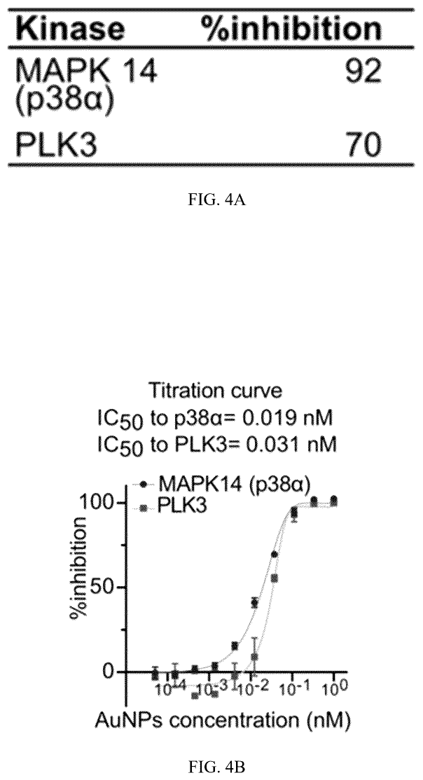

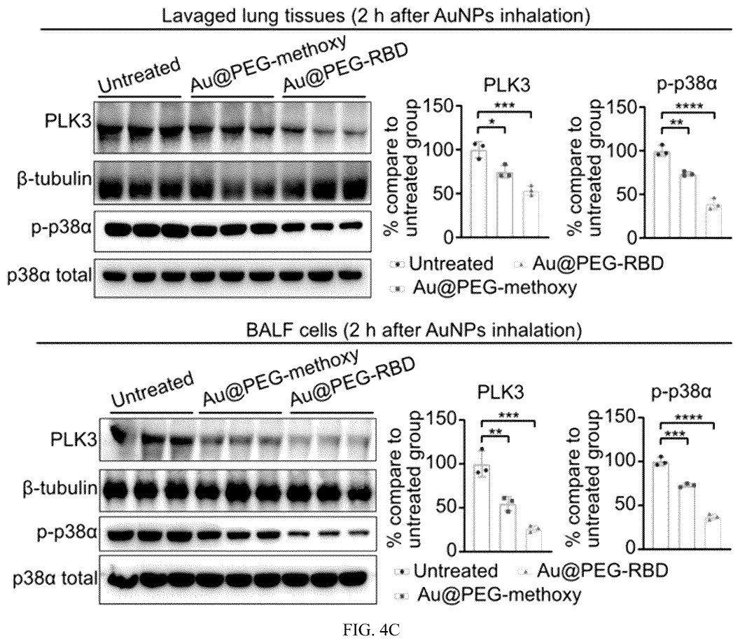

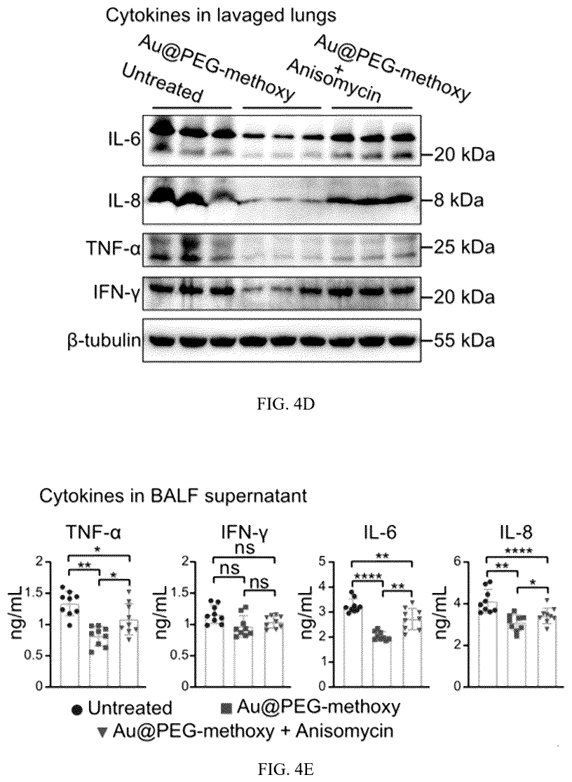

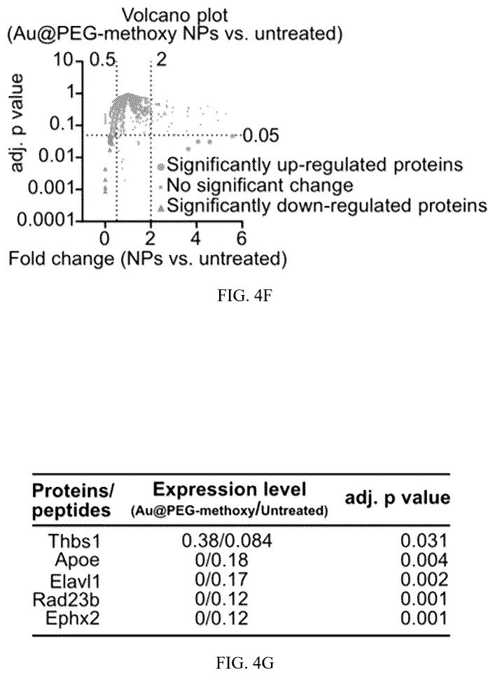

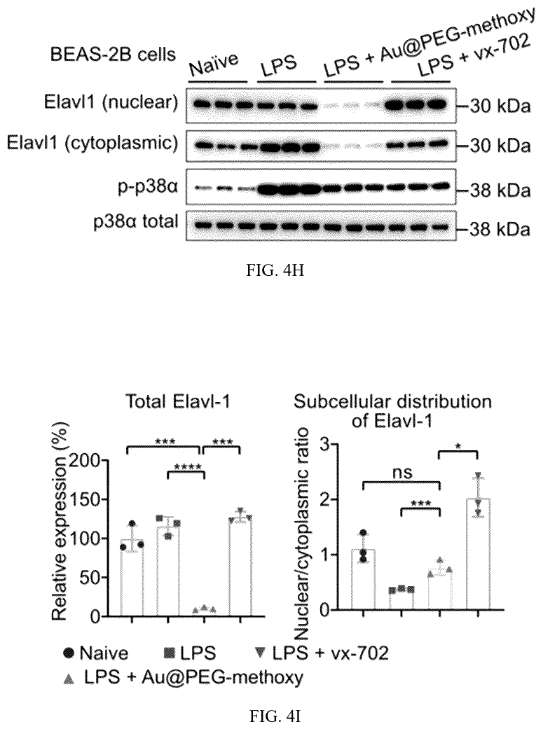

Kinome profiling unbiasedly identified potential pharmacological targets of 20-nm gold NP. Notably, ˜20 nM Au@PEG-methoxy NP inhibited only 5 out of 281 kinases by ≥70%, two ARDS-related: p38α (92% inhibition) and PLK3 (70% inhibition) (see FIG. 4A and Table 8). The half-maximal inhibitory concentrations (IC50's) of Au@PEG-methoxy NP for p38α and PLK3 were 0.019 and 0.031 nM, respectively (see FIG. 4B), indicating potent inhibition. p38α is a clinically tested drug target for ARDS; SB-681323, a p38α inhibitor, reduced serum levels of IL-6, IL-8, and TNF-α receptor in ARDS patients^{[33]}. PLK3, while not yet tested clinically, was upregulated in ARDS rodents^{[34]}; its genetic knockout prevented death caused by sepsis and its inhibition reduced kidney injury^{[36]}; accordingly, PLK3 is a likely therapeutic target for ARDS. The 20-nm gold NP did not inhibit other isoforms of p38 (p38β, p38δ, or p38γ) or other kinases in representative signaling pathways (for example, AKT-mTOR, RAF-MEK-ERK, and JAK), proof of its limited off-target effect and consistent with the data for 3-nm gold NP^{[37]}. Western blot verified that Au@PEG-RBD and Au@PEG-methoxy NPs inhibited PLK3 and p38α phosphorylation (p-p38α) in BALF cells and lavaged lung tissues upon inhalation by LPS-induced ARDS hamsters, with the former yielding faster inhibition (see FIG. 4C). Another rescue study showed that intratracheal instillation of anisomycin, a p38α activator, prior to Au@PEG-RBD NP inhalation abrogated its efficacy; western blot and ELISA analysis of lavaged lungs and BALF cells revealed no cytokine inhibition (see FIGS. 4D and 4E), verifying that the efficacy of gold NPs originates from p38α inhibition. These results match our past report of p38α inhibition by 3-nm gold NPs in the fibrotic kidneys (There is no commercial activator to validate the role of PLK3 by a similar rescue study.)

Next, unbiased proteomics analysis identified changes in protein expression in the ARDS lungs following Au@PEG-methoxy NP treatment. Of the 2488 proteins screened, we detected only 26 differentially expressed proteins (DEPs) in “Au@PEG-methoxy NP” group with ≥2-fold higher or ≥50%-lower expression levels than the untreated group (P<0.05). The 26 DEPs were too few to enrich Kyoto Encyclopedia of Genes and Genomes pathways or reveal gene ontology terms (see FIG. 4F), reinforcing the specificity of gold NP in targeting ARDS inflammation. Still, we identified DEPs related to ARDS, including upregulation of Thbs1 (which protects lung injury^{[38]}), downregulation of Ephx2^{[39]} and Srsf1^{[40]} (therapeutic targets of ARDS), downregulation of Elavl1^{[41]} and Sod2^{[42]} (upregulated in ARDS), and downregulation of Pclb3^{[43]} and Strap^{[44]} (positive modulators of IL-8, IL-6, or TNF-α) (see FIG. 4G and Table 9). Incidentally, Elavl1, a downstream nuclear factor of p38α,38 binds to the mRNA of inflammatory cytokines and promotes their protein expression, and p38α inhibition hampers the nucleus-to-cytosol translocation of Elavl1 and inhibits cytokines^{[45,46]} Here, Au@PEG-methoxy NP inhibited total Elavl1 (nucleus and cytosol) in LPS-stimulated BEAS-2S bronchial epithelial cells and moderately blocked the nucleus-to-cytosol translocation of Elavl1; conversely, vx702, a p38α inhibitor, blocked such translocation strongly without inhibiting total Elavl1 (see FIGS. 4H and 4I). Thus, Au@PEG-methoxy NP blocks Elavl1 nucleus-to-cytosol translocation as a p38α inhibitor and may directly interact with Elavl1 independently of p38α.

### EXAMPLE 4—LONG-TERM TOXICITY OF INHALED Au@PEG-RBD NPs

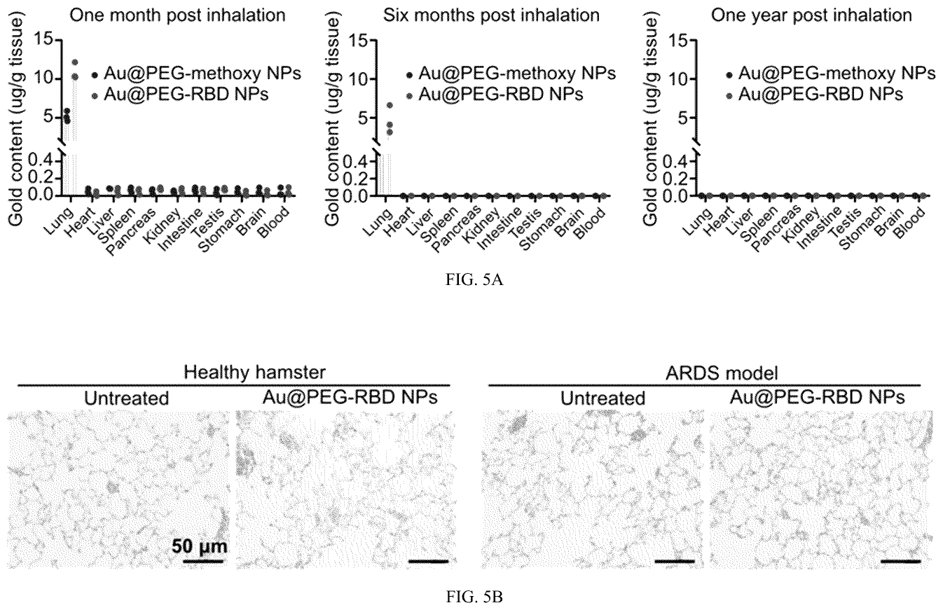

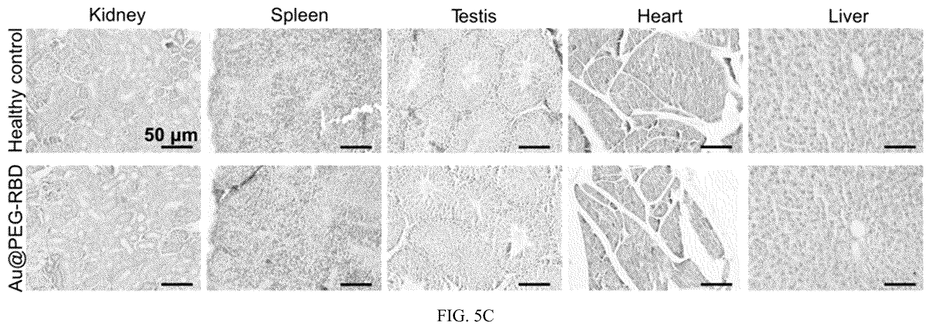

In the lungs of Au@PEG-RBD NP-treated ARDS hamsters, the gold contents remained abundant after 1 month, dropped by half after 6 months, and became undetectable after 12 months (see FIG. 5A). There was no detectable gold in other major organs across all time points. Tissue morphology of the lungs and major organs by histological examination (see FIGS. 5B and 5C), blood cell counts, liver and kidney functions, and total serum protein (see Table 10) were largely normal after 12 months. Only serum lactate dehydrogenase level was slightly higher than normal after 1 month (probably due to tissue repair^{[25]}), but it later returned to normal. The results indicate the long-term clearance and limited in vivo toxicity of Au@PEG-RBD NP.

### EXAMPLE 5—EFFICACY OF Au@PEG-RBD NPs IN HCL-INDUCED ARDS HAMSTERS

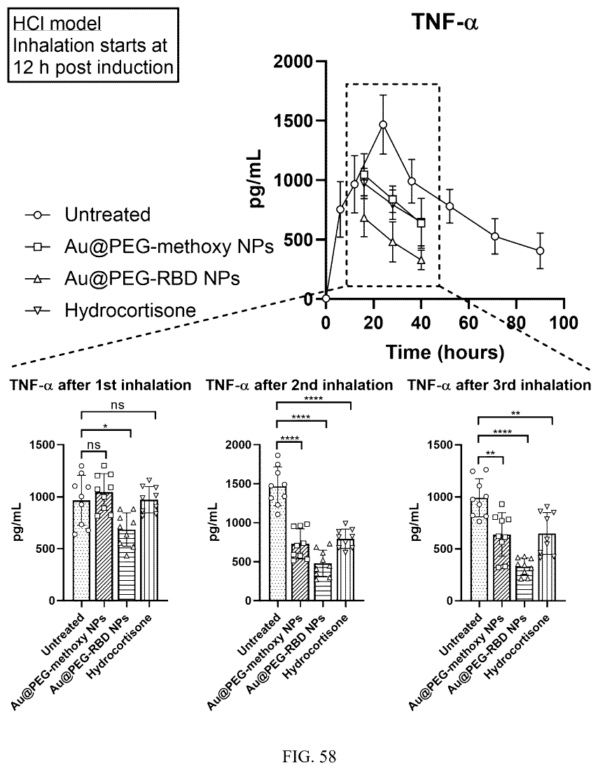

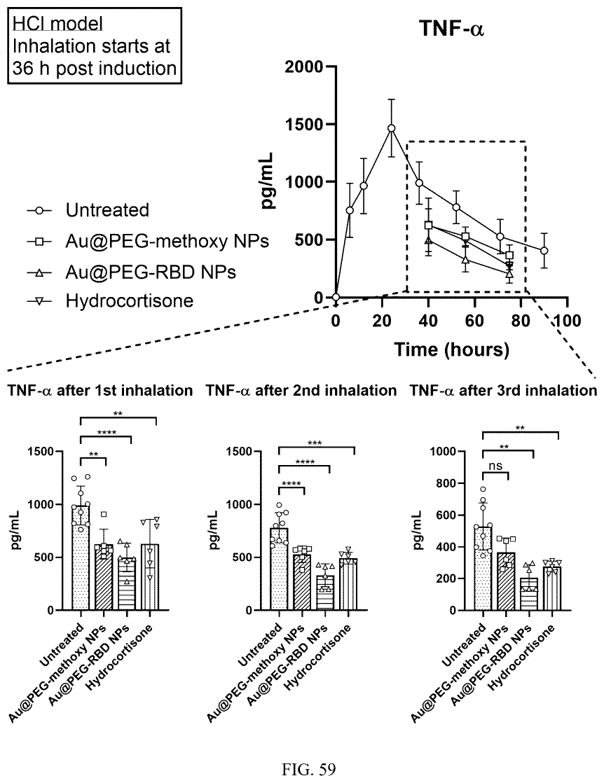

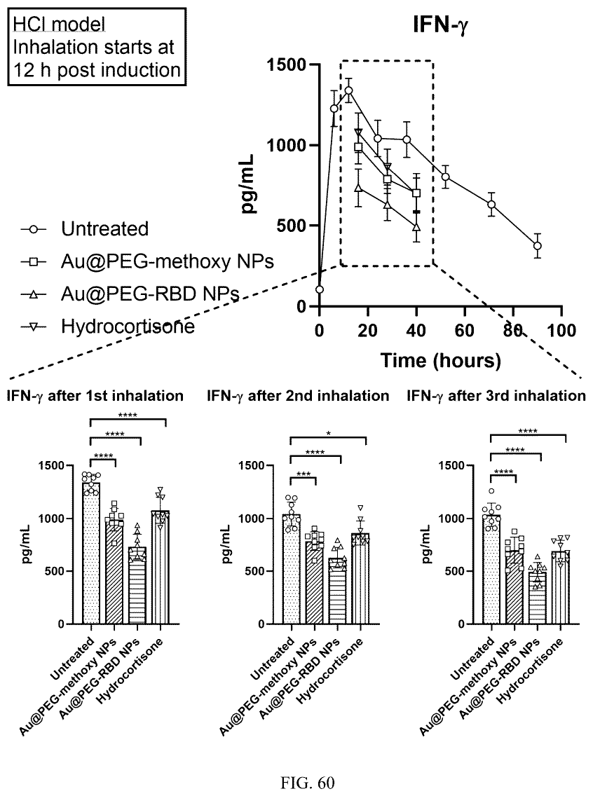

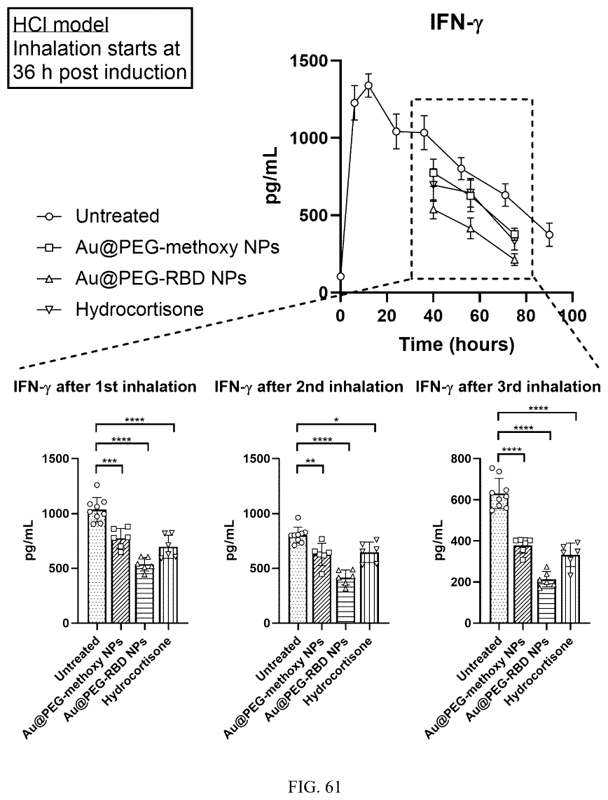

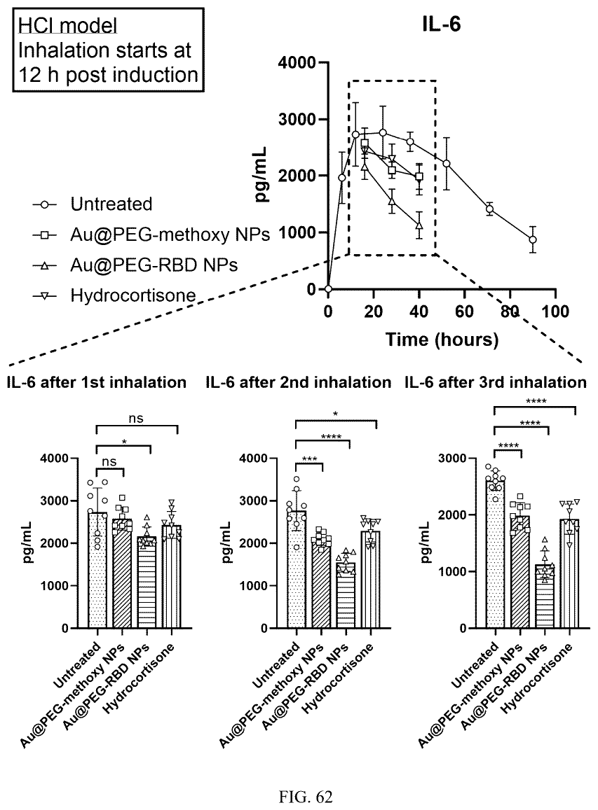

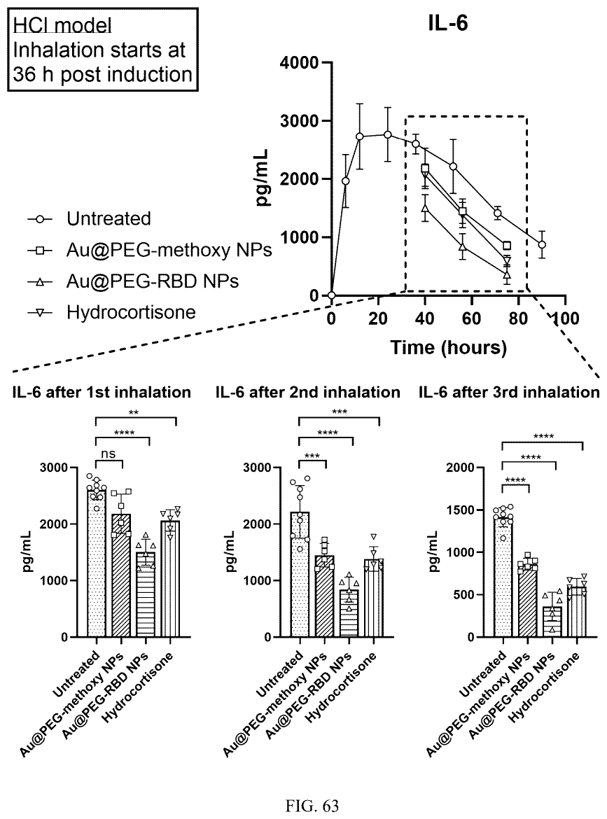

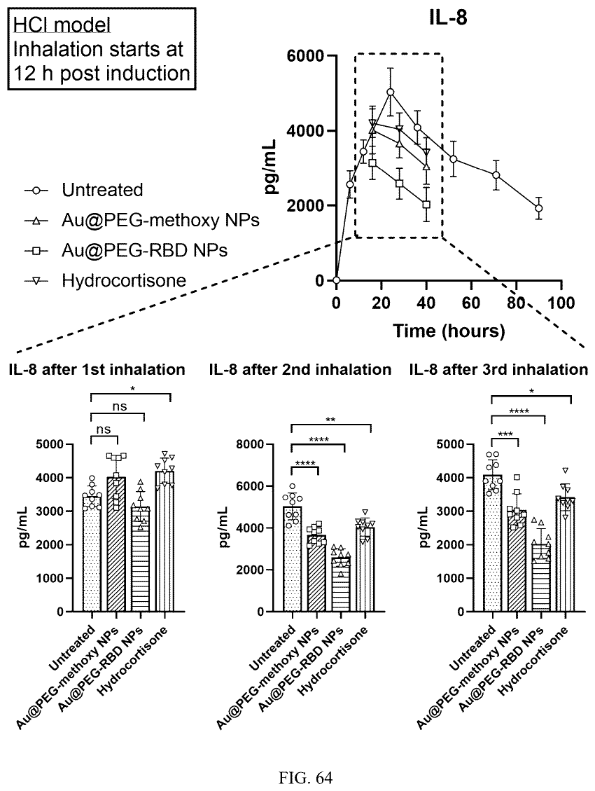

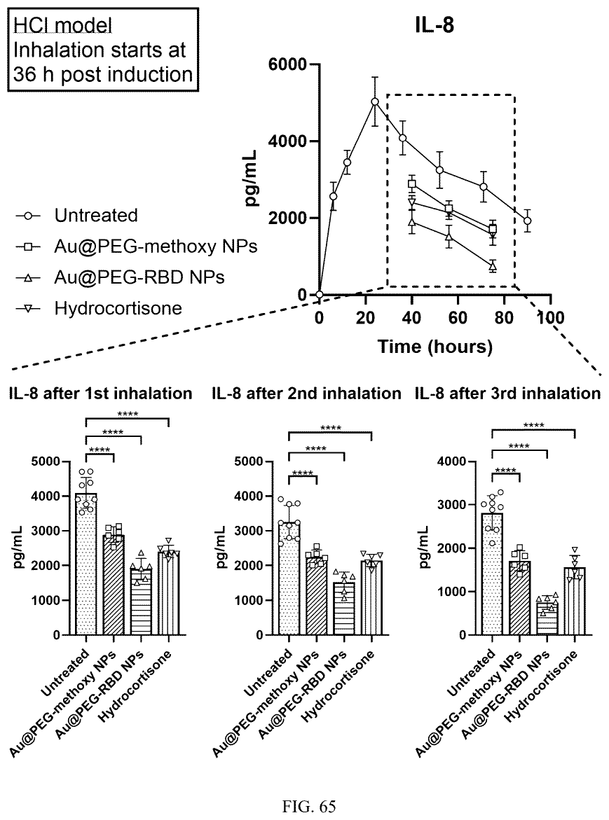

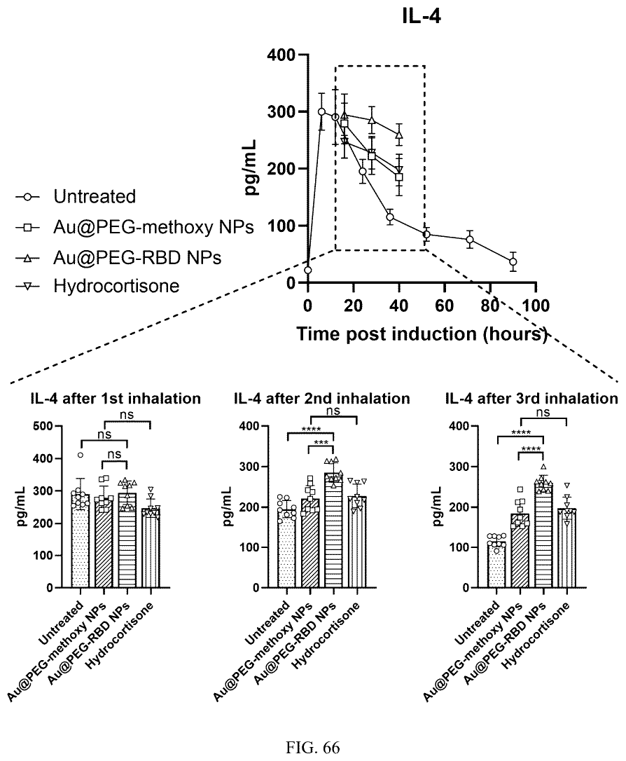

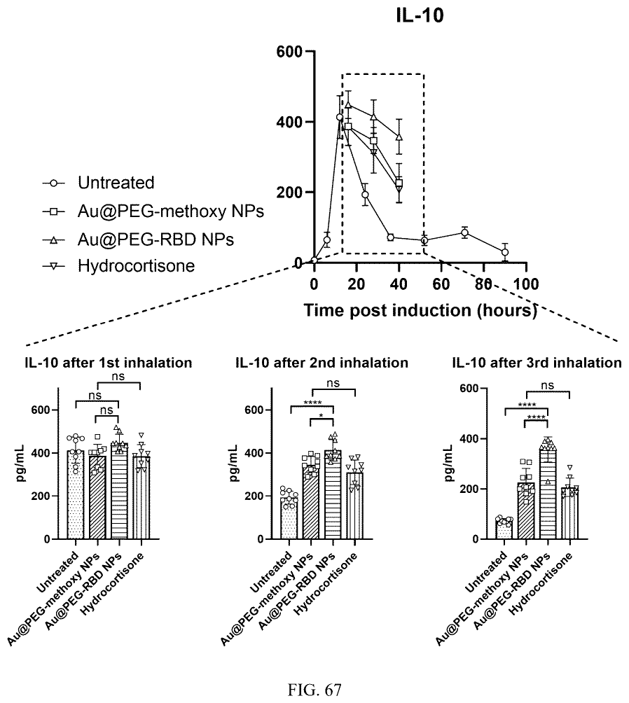

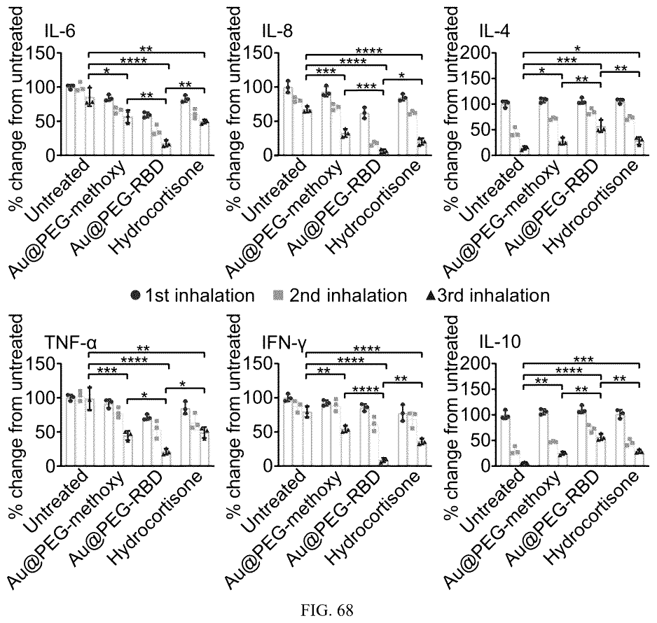

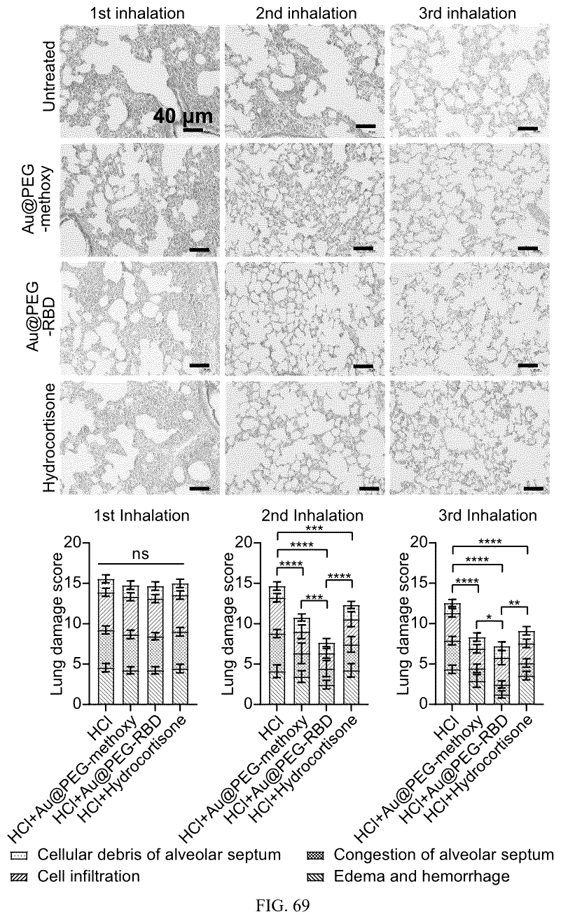

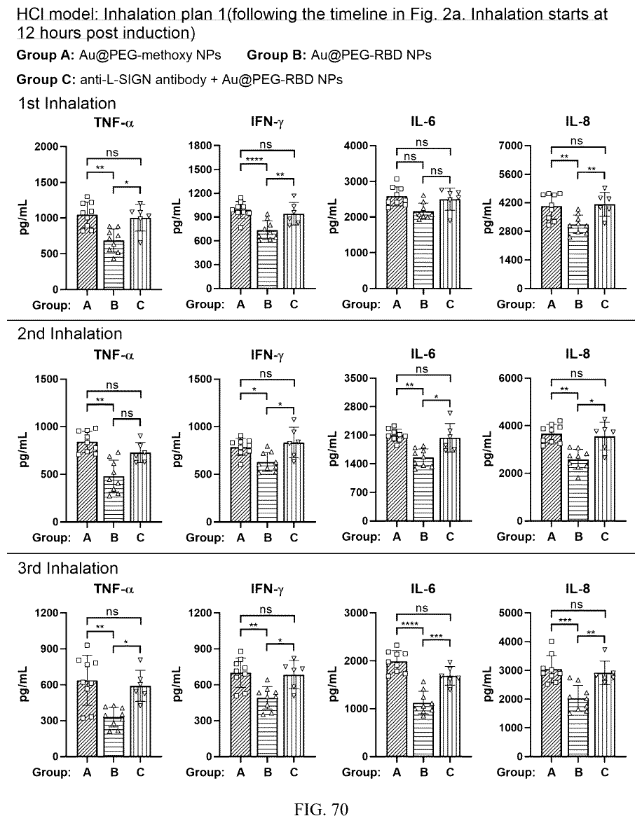

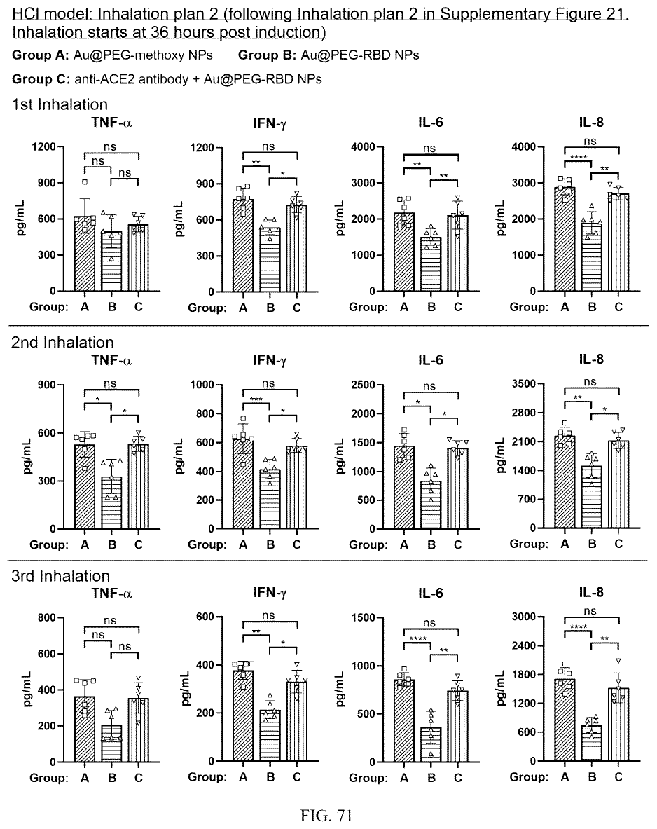

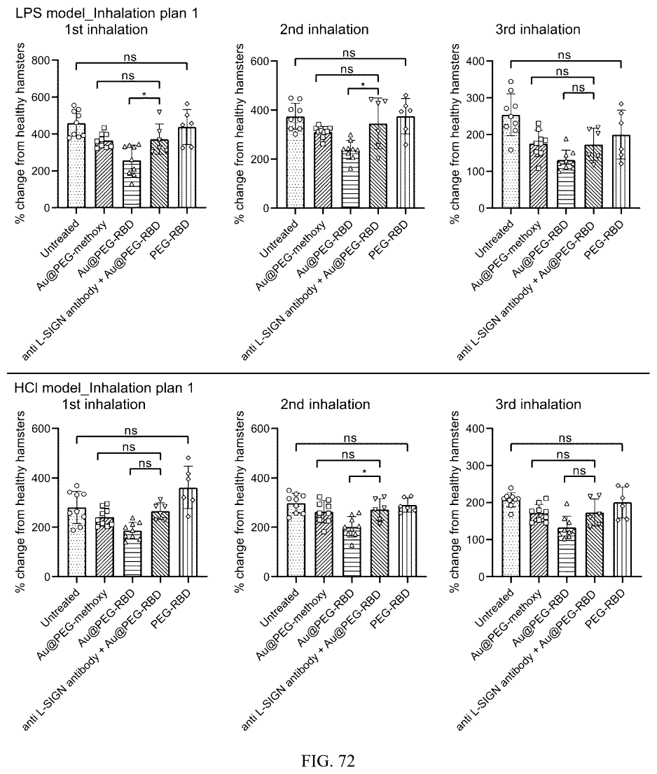

We validated the therapeutic efficacy of Au@PEG-RBD NP in a second hamster disease model. HCl-induced ARDS mimics the aspiration of stomach contents into the lungs, another common cause^{[20]}. LPS and HCl induction caused similar inflammatory responses, in terms of the kinetics and degree of (i) upregulation of ACE2 and L-SIGN and (ii) proinflammatory cytokines in the whole ARDS lung (see FIG. 6A). Following the same treatment regimen (see FIG. 2A), Au@PEG-RBD NP downregulated proinflammatory cytokines and upregulated anti-inflammatory cytokines in BALF supernatant (see FIG. 6B and FIGS. 58-67) and lavaged lung tissues (see FIG. 6C and FIG. 68), ROS levels in BALF cells (FIG. 6D), and lung tissue injury (see FIG. 69) most effectively in HCl-induced ARDS hamsters. Also, intratracheal blocking with antibodies against L-SIGN reversed the efficacy of Au@PEG-RBD NP to a similar level of Au@PEG-methoxy NP in HCl-induced ARDS hamsters (see FIG. 6E, FIGS. 7O and 7I). Au@PEG-RBD NP exhibited efficacy faster than Au@PEG-methoxy NP and hydrocortisone (1 vs. 3 administrations).

According to the embodiments of the subject invention, the key discovery is the identification of the time-dependent, transient upregulation of ACE2 and L-SIGN in ARDS lungs upon disease onset. This unique pathophysiological insight inspires the design of Au@PEG-RBD NP for enhancing delivery to lung cells that overexpress ACE2 and L-SIGN.

The NPs according to the embodiments of the subject invention differ from previous ARDS nanomedicines, which use ligands for targeting endothelial cells (for example, adhesion molecules) or immune cell membrane (for example, macrophage) for homing to inflamed sites as shown in Table 1. When combined with inhalation (a less invasive route of delivery than instillation³⁶ and intraperitoneal injection³⁷), the NP distributes homogenously in the lung tissue and accelerates NP accumulation in ARDS lungs. Another major finding is that 20-nm gold cores, rather than Spike RBD or PEG, serve as therapeutic agents for ARDS, requiring no additional chemical drugs, biologics, or physical forces.

The embodiments of the subject invention contribute to the growing list of self-therapeutic applications of gold NPs, from treating psoriasis³⁸ to addressing kidney fibrosis³⁴. In ARDS, previous reports have primarily used gold NPs merely as drug carriers, for example, P12 peptide for blocking Toll-like receptors²², curcumin as an anti-oxidant³⁹, and dexamethasone as anti-inflammatory agent⁴⁰. Beyond their general, well-established anti-inflammatory⁴¹ and anti-oxidative⁴² properties, the gold NPs of the subject invention contributes to pinning down two therapeutic targets of gold NPs in ARDS, one clinically tested (p38α) and another an emerging target (PLK3).

The results of the current investigation will catalyze the translation of gold NPs for alleviating ARDS. Firstly, past gold nanomedicines were preventive and injected before disease induction⁴³. Herein, the hamsters inhale NPs 12 hours post-disease induction, at the peak of ARDS severity as shown in FIG. 3A and FIG. 6B. This timing is clinically relevant, as the acute nature of ARDS makes timely diagnosis and prevention challenging.

Next, Au@PEG-RBD NPs are not retained in the lungs or other major organs one year post-inhalation, mitigating concerns over the toxicity of classical gold ion-based therapies⁴⁴. Further research is warranted to understand how gold NPs are cleared from the lungs.

Finally, gold NP offers a more convenient chemical handle for tracking in vivo distribution compared to classical organic NPs (for example, liposome and micelle). Distribution studies, which have previously focused on anti-ARDS efficacy, now reveal useful insights into lung-NP interactions. Herein, ICP-MS and confocal reflectance imaging are employed to quantify and visualize the distribution of gold NPs at the organ and tissue levels, respectively, as shown in FIGS. 2B-2E and FIG. 5A.

At the cellular level, IHC and confocal reflectance imaging are combined to prove the entry of NPs to specific lung cell types. While flow cytometry may empower quantification of gold NPs in different lung cell types as shown in mice¹⁹, similar studies are currently infeasible due to the scarcity of commercially available hamster-reactive antibodies⁴⁵.

According to the embodiments of the subject invention, NPs demonstrate anti-inflammation efficacy against severe ARDS, are easy to synthesize, exhibit anti-ARDS efficacy without requiring drug loading, and do not exhibit significant outside-pulmonary retention and systemic side effects.

Compared to the existing technology, the non-invasive inhalable NPs of the subject invention locally target lung cells with more homogenous distribution. Overall, this approach provides a safer and more effective solution for lung delivery and ARDS management.

### CONCLUSION

We present a disease-driven, non-invasive gold nanomedicine for ARDS. It hinges upon our discovery of the transient upregulation of ACE2 and L-SIGN on the lung epithelium soon after ARDS onset (12-36 h). We let ARDS pathophysiology inform the dose regimen by judiciously aligning dosing with the timepoint at which ACE2 or L-SIGN is upregulated by ARDS. That is, L-SIGN predominantly contributes to the lung delivery of Au@PEG-RBD NP ˜12 h post-ARDS onset whereas ACE2 predominantly contributes to delivery ˜36 h post-ARDS onset; both receptors act additively to mediate lung targeting. Accordingly, we achieve rapid delivery to the inflamed endothelium but not so prolonged that long-term accumulation may become a safety concern. The inhalable NP supports widespread distribution to the ARDS lung tissue and effective targeting of the epithelium, yielding efficacy within six total hours of inhalation. As a treatment, we commence the first dose when the pro-inflammatory response peaks in the ARDS lungs upon disease establishment (12 h post-induction) (FIGS. 3 and 6), contrary to past preventive studies that began 4-6 h post-ARDS induction (Tables 1 and 2). This point is clinically relevant for acute conditions like ARDS where timely prediction is challenging. The NP is not retained in the lungs or other major organs 1 year post-inhalation, mitigating concerns over long-term toxicity^{[47]}.

Another major finding is that gold NPs are self-therapeutic for ARDS, adding to their emerging use for other important diseases, such as psoriasis^{[48]} and kidney fibrosis^{[37]}. In ARDS, past reports used gold NPs merely as drug carriers, say P12 peptide to block Toll-like receptor^{[29]}, curcumin as an antioxidant^{[49]}, and dexamethasone as an anti-inflammatory agent^{[50]}. Our contribution lies in pinning down two therapeutic targets of gold NP in ARDS, one clinically tested (p38α) and another with preclinical evidence (PLK3), shedding light to the therapeutic mechanism of gold NP beyond its established anti-inflammatory^{[51]} and anti-oxidative^{[52]} properties. Notably, Au@PEG-RBD NP reduces ARDS more effectively than corticosteroid, a key standard of care with clinically validated efficacy against ARDS (Table 11). Most reported clinical outcomes of corticosteroid treatment were based on survival data, ventilation-free days, and length of stay at the intensive care unit, often not reporting changes in basic pathological markers. Still, a few clinical studies reported pathological markers besides clinical outcomes, showing reduced serum IL-6 level^{[53,54]} and lung injury score^{[55,56]} on Day 3 post-treatment with hydrocortisone and methylprednisolone on ARDS patients. Such past clinical results match our preclinical efficacy data that showed lowered IL-6 levels in the lung (BALF and lavaged lung tissue) and lung tissue injury score after three administrations of Au@PEG-RBD NP and, to a lesser extent, hydrocortisone (over a total duration of 30 h) on the two ARDS hamster models tested.

For the limitation of this study, we used prokaryotes to produce the Spike RBD proteins when constructing this lung nanomedicine because (i) fast and economical protein expression as grams of RBD proteins were required (Table 4) and (ii) the affinities of the prokaryotic RBD product to ACE2 and L-SIGN were only slightly less than those of commercial eukaryotic RBD (FIGS. 13A-13D). For clinical translation, however, protein expression should involve a mammalian system to avoid allergic response to humans. Next, our treatment regimen entails three inhalation sessions of 2 h each, a practical duration for patients with severe ARDS because they are often intubated and thus sedated and bed bound^{[57]}. To shorten the inhalation duration (e.g., ˜20 min) for improved patient compliance, we will need to prepare a more concentrated NP liquid stock at a larger reaction scale for aerosolization while addressing the technical challenge of protein-induced NP aggregation. Further, the scarcity of commercial hamster-reactive antibodies made the use of flow cytometry^{[25]} for quantifying the distribution of inhaled NPs to various lung cell types challenging^{[58]}. Finally, follow-up validation studies on the efficacy and toxicology of our Spike-conjugated gold NP in large animal models are needed before clinical trials.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1 Ranieri, V. M. et al. Acute respiratory distress syndrome: the Berlin Definition. Journal of the American Medical Association 307, 2526-2533 (2012). https://doi.org/10.1001/jama.2012.5669
2 Matthay, M. A. et al. Acute respiratory distress syndrome. Nature Reviews Disease Primers 5, 18 (2019). https://doi.org/10.1038/s41572-019-0069-0
3 Griffiths, M. J. D. et al. Guidelines on the management of acute respiratory distress syndrome. BMJ Open Respiratory Research 6, e000420 (2019). https://doi.org/10.1136/bmjresp-2019-000420
4 Bernard, G. R. et al. High-dose corticosteroids in patients with the adult respiratory distress syndrome. New England Journal of Medicine 317, 1565-1570 (1987). https://doi.org/10.1056/nejm198712173172504
5 Steinberg, K. P. et al. Efficacy and safety of corticosteroids for persistent acute respiratory distress syndrome. New England Journal of Medicine 354, 1671-1684 (2006). https://doi.org/10.1056/NEJMoa051693
6 Bajwa, E. K., Malhotra, C. K., Thompson, B. T., Christiani, D. C. & Gong, M. N. Statin therapy as prevention against development of acute respiratory distress syndrome: an observational study. Critical Care Medicine 40, 1470-1477 (2012). https://doi.org/10.1097/CCM.0b013e3182416d7a
7 Frank, A. J. & Thompson, B. T. Pharmacological treatments for acute respiratory distress syndrome. Current Opinion in Critical Care 16, 62-68 (2010). https://doi.org/10.1097/MCC.0b013e328334b151
8 Watkins, T. R. et al. in D16. Epidemiology of Acute Lung Injury A5598 (2011).
9 Qiu, M. et al. Lung-selective mRNA delivery of synthetic lipid nanoparticles for the treatment of pulmonary lymphangioleiomyomatosis. Proceedings of the National Academy of Sciences 119, e2116271119 (2022). https://doi.org/doi: 10.1073/pnas.2116271119
10 Solé-Lleonart, C. et al. Intratracheal Administration of Antimicrobial Agents in Mechanically Ventilated Adults: An International Survey on Delivery Practices and Safety. Respiratory Care 61, 1008-1014 (2016). https://doi.org/10.4187/respcare.04519
11 Lokugamage, M. P. et al. Optimization of lipid nanoparticles for the delivery of nebulized therapeutic mRNA to the lungs. Nature Biomedical Engineering 5, 1059-1068 (2021). https://doi.org/10.1038/s41551-021-00786-x
12 Artigas, A. et al. Inhalation therapies in acute respiratory distress syndrome. Annals of Translational Medicine 5, 293 (2017).
13 Choi, H. S. et al. Rapid translocation of nanoparticles from the lung airspaces to the body. Nature Biotechnology 28, 1300-1303 (2010). https://doi.org/10.1038/nbt.1696
14 Trbojević-Akmačić, I., Petrović, T. & Lauc, G. SARS-COV-2 S glycoprotein binding to multiple host receptors enables cell entry and infection. Glycoconjugate Journal 38, 611-623 (2021). https://doi.org/10.1007/s10719-021-10021-z
15 Kesidis, A. et al. Expression of eukaryotic membrane proteins in eukaryotic and prokaryotic hosts. Methods 180, 3-18 (2020). https://doi.org/https://doi.org/10.1016/j.ymeth.2020.06.006
16 Laue, M. et al. Morphometry of SARS-COV and SARS-COV-2 particles in ultrathin plastic sections of infected Vero cell cultures. Scientific reports 11, 3515 (2021). https://doi.org/10.1038/s41598-021-82852-7
17 Castranova, V., Rabovsky, J., Tucker, J. H. & Miles, P. R. The alveolar type II epithelial cell: a multifunctional pneumocyte. Toxicology and applied pharmacology 93, 472-483 (1988). https://doi.org/10.1016/0041-008x(88) 90051-8
18 FDA, U.S. Estimating the Maximum Safe Starting Dose in Initial Clinical Trials for Therapeutics in Adult Healthy Volunteers <https://www.fda.gov/regulatory-information/search-fda-guidance-documents/estimating-maximum-safe-starting-dose-initial-clinical-trials-therapeutics-adult-healthy-volunteers> (2005).
19 Yin, B. et al. Intrapulmonary cellular-level distribution of inhaled nanoparticles with defined functional groups and its correlations with protein corona and inflammatory response. ACS Nano 13, 14048-14069 (2019). https://doi.org/10.1021/acsnano.9b06424
20 Andersen, J. T. et al. Structure-based mutagenesis reveals the albumin-binding site of the neonatal Fc receptor. Nature communications 3, 610 (2012). https://doi.org/10.1038/ncomms1607
21 Schnitzer, J. E. & Oh, P. Antibodies to SPARC inhibit albumin binding to SPARC, gp60, and microvascular endothelium. Am J Physiol 263, H1872-1879 (1992). https://doi.org/10.1152/ajpheart.1992.263.6.H1872
22 Xiong, Y. et al. Peptide-gold nanoparticle hybrids as promising anti-inflammatory nanotherapeutics for acute lung injury: in vivo efficacy, biodistribution, and clearance. Advanced Healthcare Materials 7, e1800510 (2018). https://doi.org/10.1002/adhm.201800510
23 Qu, Y. et al. Thrombospondin-1 protects against pathogen-induced lung injury by limiting extracellular matrix proteolysis. JCI Insight 3 (2018). https://doi.org/10.1172/jci.insight.96914
24 Zhang, J. et al. Macrophage Inactivation by Small Molecule Wedelolactone via Targeting sEH for the Treatment of LPS-Induced Acute Lung Injury. ACS Central Science 9, 440-456 (2023). https://doi.org/10.1021/acscentsci.2c01424
25 Fu, C., Hu, Y., Liu, J. & Yang, Y. The splicing factor SRSF1 stabilizes the mRNA of TSLP to enhance acute lung injury. Cellular Immunology 362, 104299 (2021). https://doi.org/https://doi.org/10.1016/j.cellimm.2021.104299
26 Hoffman, O. et al. Detrimental ELAVL-1/HuR-dependent GSK3 mRNA stabilization impairs resolution in acute respiratory distress syndrome. PloS one 12, e0172116 (2017). https://doi.org/10.1371/journal.pone.0172116
27 Huang, C. et al. MicroRNA and mRNA expression profiling in rat acute respiratory distress syndrome. BMC Medical Genomics 7, 46 (2014). https://doi.org/10.1186/1755-8794-7-46
28 Bezzerri, V. et al. Phospholipase C-β3 Is a Key Modulator of IL-8 Expression in Cystic Fibrosis Bronchial Epithelial Cells. The Journal of Immunology 186, 4946-4958 (2011). https://doi.org/10.4049/jimmunol.1003535
29 Huh, H. D. et al. STRAP Acts as a Scaffolding Protein in Controlling the TLR2/4 Signaling Pathway. Scientific reports 6, 38849 (2016). https://doi.org/10.1038/srep38849
30 Christie, J. D. et al. A randomized dose-escalation study of the safety and anti-inflammatory activity of the p38 mitogen-activated protein kinase inhibitor Dilmapimod in severe trauma subjects at risk for acute respiratory distress syndrome. Critical Care Medicine 43, 1859-1869 (2015). https://doi.org/10.1097/ccm.0000000000001132
31 Blázquez-Prieto, J. et al. Activation of p21 limits acute lung injury and induces early senescence after acid aspiration and mechanical ventilation. Translational Research 233, 104-116 (2021). https://doi.org/10.1016/j.trsl.2021.01.008
32 Kostyak, J. C., Sarojini, S. S. & Naik, U. P. Plk3 deletion eliminates bacteremia in an in vivo model of sepsis. Blood 140, 8363-8364 (2022). https://doi.org/10.1182/blood-2022-160291
33 Zhu, D. et al. Liposome-mediated biomimetic delivery of Plk3 inhibitor with NIR II-triggered release prevents renal ischemia-reperfusion injury. Advanced Therapeutics 5, 2200087 (2022). https://doi.org/https://doi.org/10.1002/adtp.202200087
34 Chan, C. K. W. et al. A sub-10-nm, folic acid-conjugated gold nanoparticle as self-therapeutic treatment of tubulointerstitial fibrosis. Proceedings of the National Academy of Sciences 120, e2305662120 (2023). https://doi.org/doi: 10.1073/pnas.2305662120
35 Drent, M., Cobben, N. A., Henderson, R. F., Wouters, E. F. & van Dieijen-Visser, M. Usefulness of lactate dehydrogenase and its isoenzymes as indicators of lung damage or inflammation. The European respiratory journal 9, 1736-1742 (1996). https://doi.org/10.1183/09031936.96.09081736
36 Gao, W. et al. Size-dependent anti-inflammatory activity of a peptide-gold nanoparticle hybrid in vitro and in a mouse model of acute lung injury. Acta Biomaterialia 85, 203-217 (2019). https://doi.org/10.1016/j.actbio.2018.12.046
37 dos Santos Haupenthal, D. P. et al. Effects of treatment with gold nanoparticles in a model of acute pulmonary inflammation induced by lipopolysaccharide. Journal of Biomedical Materials Research Part A 108, 103-115 (2020). https://doi.org/https://doi.org/10.1002/jbm.a.36796
38 Han, R. et al. Alkyl-terminated gold nanoparticles as a self-therapeutic treatment for psoriasis. Nano Letters. 21, 8723-8733 (2021). https://doi.org/10.1021/acs.nanolett.1c02899
39 Casagrande, L. d. R. et al. Green synthesis of gold nanoparticles modulates lipopolysaccharide-induced lung inflammation in Wistar rats. Basic & Clinical Pharmacology & Toxicology 132, 472-484 (2023). https://doi.org/https://doi.org/10.1111/bcpt.13854
40 Li, C. et al. Co-delivery of Dexamethasone and a MicroRNA-155 Inhibitor Using Dendrimer-Entrapped Gold Nanoparticles for Acute Lung Injury Therapy. Biomacromolecules 22, 5108-5117 (2021). https://doi.org/10.1021/acs.biomac.1c01081
41 Sumbayev, V. V. et al. Gold Nanoparticles Downregulate Interleukin-1β-Induced Pro-Inflammatory Responses. Small 9, 472-477 (2013). https://doi.org/https://doi.org/10.1002/smll.201201528
42 BarathManiKanth, S. et al. Anti-oxidant effect of gold nanoparticles restrains hyperglycemic conditions in diabetic mice. Journal of Nanobiotechnology 8, 16 (2010). https://doi.org/10.1186/1477-3155-8-16
43 Wang, L. et al. Manipulation of macrophage polarization by peptide-coated gold nanoparticles and its protective effects on acute lung injury. Journal of Nanobiotechnology 18, 38 (2020). https://doi.org/10.1186/s12951-020-00593-7
44 Balfourier, A., Kolosnjaj-Tabi, J., Luciani, N., Carn, F. & Gazeau, F. Gold-based therapy: From past to present. Proceedings of the National Academy of Sciences 117, 22639-22648 (2020). https://doi.org/doi: 10.1073/pnas.2007285117
45 Warner, B. M., Safronetz, D. & Kobinger, G. P. in Emerging and Re-emerging Viral Infections: Advances in Microbiology, Infectious Diseases and Public Health Volume 6 (eds Giovanni Rezza & Giuseppe Ippolito) 87-101 (Springer International Publishing, 2017).
46 Turkevich, J., Stevenson, P. C. & Hillier, J. A study of the nucleation and growth processes in the synthesis of colloidal gold. Discussions of the Faraday Society 11, 55-75 (1951). https://doi.org/10.1039/DF9511100055
47 Haiss, W., Thanh, N. T. K., Aveyard, J. & Fernig, D. G. Determination of Size and Concentration of Gold Nanoparticles from UV-Vis Spectra. Analytical Chemistry 79, 4215-4221 (2007). https://doi.org/10.1021/ac0702084
48 Matute-Bello, G., Frevert, C. W. & Martin, T. R. Animal models of acute lung injury. American journal of physiology. Lung cellular and molecular physiology 295, L379-399 (2008). https://doi.org/10.1152/ajplung.00010.2008
49 Chen, X. Y. et al. Creation of lung-targeted dexamethasone immunoliposome and its therapeutic effect on bleomycin-induced lung injury in rats. PloS one 8, e58275 (2013). https://doi.org/10.1371/journal.pone.0058275
50 Howard, M. D., Greineder, C. F., Hood, E. D. & Muzykantov, V. R. Endothelial targeting of liposomes encapsulating SOD/catalase mimetic EUK-134 alleviates acute pulmonary inflammation. Journal of Controlled Release 177, 34-41 (2014). https://doi.org/10.1016/j.jconrel.2013.12.035
51 Wang, Z., Li, J., Cho, J. & Malik, A. B. Prevention of vascular inflammation by nanoparticle targeting of adherent neutrophils. Nature Nanotechnology 9, 204-210 (2014). https://doi.org/10.1038/nnano.2014.17
52 Chu, D., Gao, J. & Wang, Z. Neutrophil-mediated delivery of therapeutic nanoparticles across blood vessel barrier for treatment of inflammation and infection. ACS Nano 9, 11800-11811 (2015). https://doi.org/10.1021/acsnano.5b05583
53 Spence, S. et al. Targeting Siglecs with a sialic acid-decorated nanoparticle abrogates inflammation. Science Translational Medicine 7, 303ra140 (2015). https://doi.org/10.1126/scitranslmed.aab3459
54 Xu, P. T. et al. Cerium oxide nanoparticles: a potential medical countermeasure to mitigate radiation-induced lung injury in CBA/J mice. Radiation Research. 185, 516-526 (2016). https://doi.org/10.1667/rr14261.1
55 Li, S. J. et al. Targeting delivery of simvastatin using ICAM-1 antibody-conjugated nanostructured lipid carriers for acute lung injury therapy. Drug Delivery. 24, 402-413 (2017). https://doi.org/10.1080/10717544.2016.1259369
56 Li, N. et al. Surfactant protein-A nanobody-conjugated liposomes loaded with methylprednisolone increase lung-targeting specificity and therapeutic effect for acute lung injury. Drug Delivery. 24, 1770-1781 (2017). https://doi.org/10.1080/10717544.2017.1402217
57 Gao, J., Wang, S. & Wang, Z. High yield, scalable and remotely drug-loaded neutrophil-derived extracellular vesicles (EVs) for anti-inflammation therapy. Biomaterials 135, 62-73 (2017). https://doi.org/10.1016/j.biomaterials.2017.05.003
58 Zhu, Y. et al. Porous Se@SiO(2) nanospheres treated paraquat-induced acute lung injury by resisting oxidative stress. International Journal of Nanomedicine. 12, 7143-7152 (2017). https://doi.org/10.2147/ijn.S143192
59 Li, S. et al. Anti-ICAM-1 antibody-modified nanostructured lipid carriers: a pulmonary vascular endothelium-targeted device for acute lung injury therapy. Journal of Nanobiotechnology 16, 105 (2018). https://doi.org/10.1186/s12951-018-0431-5
60 Jiang, S. et al. Combined delivery of angiopoietin-1 gene and simvastatin mediated by anti-intercellular adhesion molecule-1 antibody-conjugated ternary nanoparticles for acute lung injury therapy. Nanomedicine. 15, 25-36 (2019). https://doi.org/10.1016/j.nano.2018.08.009
61 Liu, Y., Yang, B., Zhao, X., Xi, M. & Yin, Z. E-Selectin-binding peptide-modified bovine serum albumin nanoparticles for the treatment of acute lung injury. AAPS PharmSciTech 20, 270 (2019). https://doi.org/10.1208/s12249-019-1403-2
62 Molinaro, R. et al. Macrophage-derived nanovesicles exert intrinsic anti-inflammatory properties and prolong survival in sepsis through a direct interaction with macrophages. Nanoscale 11, 13576-13586 (2019). https://doi.org/10.1039/c9nr04253a
63 Shen, S. et al. Engineered nanoparticles disguised as macrophages for trapping lipopolysaccharide and preventing endotoxemia. Biomaterials 189, 60-68 (2019). https://doi.org/10.1016/j.biomaterials.2018.10.029
64 Zhang, C. et al. Exosome-delivered syndecan-1 rescues acute lung injury via a FAK/p190RhoGAP/RhoA/ROCK/NF-κB signaling axis and glycocalyx enhancement. Experimental Cell Research 384, 111596 (2019). https://doi.org/10.1016/j.yexcr.2019.111596
65 Zhang, C. Y. et al. pH-responsive nanoparticles targeted to lungs for improved therapy of acute lung inflammation/injury. ACS Applied Materials & Interfaces 11, 16380-16390 (2019). https://doi.org/10.1021/acsami.9b04051
66 Ma, Q. et al. Calming cytokine storm in pneumonia by targeted delivery of TPCA-1 using platelet-derived extracellular vesicles. Matter 3, 287-301 (2020). https://doi.org/10.1016/j.matt.2020.05.017
67 Yang, Y. et al. Inflammation-targeting polymeric nanoparticles deliver sparfloxacin and tacrolimus for combating acute lung sepsis. Journal of Controlled Release 321, 463-474 (2020). https://doi.org/10.1016/j.jconrel.2020.02.030
68 Lee, Y. Y. et al. Long-acting nanoparticulate DNase-1 for effective suppression of SARS-COV-2-mediated neutrophil activities and cytokine storm. Biomaterials 267, 120389 (2021). https://doi.org/10.1016/j.biomaterials.2020.120389
69 Dos Santos Haupenthal, D. P. et al. Effects of treatment with gold nanoparticles in a model of acute pulmonary inflammation induced by lipopolysaccharide. Journal of Biomedical Materials Research Part A 108, 103-115 (2020). https://doi.org/10.1002/jbm.a.36796
70 Gao, W. et al. Enhanced anti-inflammatory activity of peptide-gold nanoparticle hybrids upon cigarette smoke extract modification through TLR inhibition and autophagy induction. ACS Applied Materials & Interfaces 11, 32706-32719 (2019). https://doi.org/10.1021/acsami.9b10536
71 Wang, Z. et al. Exosomes decorated with a recombinant SARS-COV-2 receptor-binding domain as an inhalable COVID-19 vaccine. Nature Biomedical Engineering 6, 791-805 (2022). https://doi.org/10.1038/s41551-022-00902-5
72 Zhuo, S.-H. et al. A chitosan-mediated inhalable nanovaccine against SARS-COV-2. Nano Research 15, 4191-4200 (2022). https://doi.org/10.1007/s12274-021-4012-9
73 Elder, E. et al. Protective immunity induced by an inhaled SARS-COV-2 subunit vaccine. Vaccine 41, 4743-4751 (2023). https://doi.org/https://doi.org/10.1016/j.vaccine.2023.06.015
74 Ye, T. et al. Inhaled SARS-COV-2 vaccine for single-dose dry powder aerosol immunization. Nature 624, 630-638 (2023). https://doi.org/10.1038/s41586-023-06809-8
75 Wang, S. et al. Inhalable hybrid nanovaccines with virus-biomimetic structure boost protective immune responses against SARS-COV-2 variants. Journal of Nanobiotechnology 22, 76 (2024). https://doi.org/10.1186/s12951-024-02345-3
76 Chan, K. K. et al. Engineering human ACE2 to optimize binding to the spike protein of SARS coronavirus 2. Science 369, 1261-1265 (2020). https://doi.org/10.1126/science.abc0870

We claim:

1. A method for preparing inhalable targeted gold nanoparticles for accelerated lung delivery and treating lung inflammation, the method comprising:
preparing plasmids;
performing plasmids transformation;
performing protein expression induction;
performing protein purification; and
preparing spike RBD-conjugated gold nanoparticles (Au@PEG-RBD NPs).

2. The method according to claim 1, wherein the preparing plasmids comprises:
mixing DH5α competent cells with the plasmids;
incubating the mixture on ice;
heat-shocking the mixture;
incubating the mixture on ice;
adding pre-warmed lysogeny broth to the mixture; and
incubating cells of the mixture with orbital shaking.

3. The method according to claim 2, further comprising spreading the transformed cells onto an LB-agar plate containing ampicillin and incubating the cells.

4. The method according to claim 3, further comprising selecting one colony of the cells and growing the selected colony in LB/ampicillin under shaking.

5. The method according to claim 4, further comprising collecting the cells by centrifugation and purifying the plasmids to obtain plasmids encoding His-tagged RBD (pET11a-RBD-8×His).

6. The method according to claim 1, wherein the performing plasmids transformation comprises:
thawing competent cells previously stored at −80° C. on ice;
adding the plasmids to the competent cells to obtain a mixture;
keeping cells of the mixture on ice;
heat-shocking the cells of the mixture in a water bath and incubating the cells again on ice; and
adding fresh LB medium and shaking the mixture.

7. The method according to claim 6, further comprising centrifuging the cells of the mixture and discarding supernatant.

8. The method according to claim 7, further comprising resuspending remaining pelleted cells.

9. The method according to claim 8, further comprising seeding bacteria onto an LB agar plate with antibiotics and incubating the bacteria.

10. The method according to claim 1, wherein the performing protein expression induction comprises:
transforming expression strain Origami B cells, which is pre-transformed with molecular chaperone plasmid pG-KJE8, by RBD expressing plasmid pET11a-RBD;
culturing the resultant on a LB agar plate;
selecting one single colony of the resultant;
adding the colony into fresh LB medium with antibiotics;
adding overnight cultures into the fresh LB medium; and
culturing the resultant until OD₆₀₀ reaches about 0.5.

11. The method according to claim 10, further comprising performing induction by adding induction buffer containing isopropyl β-D-1-thiogalactopyranoside and L-arabinose.

12. The method according to claim 11, further comprising collecting the cells by centrifugation for purification or storage at −80° C.

13. The method according to claim 1, wherein the performing protein purification comprises:
resuspending the origami B cells in Protein Purification Buffer and lysing the cells by an ultrasonication processor to yield a lysate;
centrifuging the lysate;
collecting supernatant of the lysate;
filtering the supernatant by a syringe filter;
incubating the supernatant with Ni-NTA resin to yield a lysate-resin mixture; and
transferring the lysate-resin mixture together with the resin to a blank gravity chromatography column.

14. The method according to claim 13, further comprising discarding liquid flow-through from the lysate-resin mixture and adding Washing Buffer to elute nonspecific binding proteins.

15. The method according to claim 14, further comprising eluting the RBD protein product by adding Elute Buffer and dialyzing it against Storage Buffer.

16. The method according to claim 15, further comprising determining purity and concentration of the protein product by sodium dodecyl sulfate (SDS)-polyacrylamide gel electrophoresis (PAGE) and Bradford assay, respectively.

17. The method according to claim 1, wherein the preparing spike RBD-conjugated gold nanoparticles (Au@PEG-RBD NPs) comprises:
synthesizing citrate-capped gold NPs (cit-AuNPs) having a diameter of about 20 nm;
after bringing HAuCl₄ to a boil state, adding sodium citrate under vigorous stirring and keeping the mixture boiling;
cooling the resultant down to obtain cit-AuNPs; and
adding freshly dissolved thiol (HS)-PEG₂₀ₖ-methoxy or HS-PEG₂₀ₖ-nitrilotriacetic acid (NTA) (Biochempeg), at a 1:1 molar ratio, to the cit-AuNPs solution at a total concentration of 5 PEG molecules per nm² of the NP surface to obtain a resultant mixture.

18. The method according to claim 17, further comprising stirring the resultant mixture, adding NiCl₂, and then stirring to obtain Au@PEG-NTA-Ni²⁺ NPs.

19. The method according to claim 18, further comprising adding His-tagged RBD proteins to the Au@PEG-NTA-Ni²⁺ NP solution and stirring to obtain Au@PEG-RBD NPs.

20. The method according to claim 19, further comprising dialyzing the obtained Au@PEG-RBD NPs against Nanopure water by centrifugal filtration.

21. A nanoparticle composition for inhalation delivery to lung epithelial cells, comprising:
a gold core having a diameter between approximately 3 nm and 50 nm;
a polyethylene glycol (PEG) shell coating the gold core; and
a plurality of recombinant Spike receptor binding domain (RBD) subunit proteins conjugated to the PEG shell,
wherein the nanoparticle composition has an overall size greater than 50 nm and is configured to inhibit p38α mitogen-activated protein kinase (MAPK) phosphorylation and inhibit polo-like kinase 3 (PLK3).

22. The nanoparticle composition of claim 21, wherein the overall size of the nanoparticle is approximately 94 nm.

23. The nanoparticle composition of claim 21, wherein the gold core diameter is approximately 20 nm.

24. The nanoparticle composition of claim 21, wherein the PEG shell is composed of 20,000-Da PEG strands.

25. The nanoparticle composition of claim 21, configured to engage liver/lymph node-specific intercellular adhesion molecule-3-grabbing integrin (L-SIGN) and angiotensin-converting enzyme 2 (ACE2) receptors on lung epithelial cells.

26. A method of treating Acute Respiratory Distress Syndrome (ARDS) in a subject, the method comprising intermittently inhaling a therapeutically effective amount of the nanoparticle composition of claim 21 to the lung epithelial cells of the subject.

27. The method of claim 26, wherein the ARDS is non-viral ARDS.

28. The method of claim 26, wherein the ARDS is induced by lipopolysaccharides (LPS) or hydrochloric acid (HCl) aspiration.

29. The method of claim 26, wherein the nanoparticle is therapeutically effective without a viral blockade mechanism.

30. The method of claim 26, wherein the therapeutic effect is achieved by the nanoparticle additively engaging the L-SIGN and ACE2 receptors.


SEQUENCE LISTING (3 sequences)

SEQ ID NO: 1 - protein, 14 aa, synthetic construct
PSHISKYILRWRPK

SEQ ID NO: 2 - DNA, 675 bp, synthetic construct
  source 1..675: mol_type = other DNA
atgaagaccatcatcgccctgagctacatcttctgcctggtgttcgccactaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattacttgtatcgcctgtttcgaaagagcaatttgaagccttttgagcgggatatttcaaccgaaatttaccaagcaggcagtacgccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaagggatccggtcaccatcaccaccaccatcatcattga

SEQ ID NO: 3 - DNA, 675 bp, synthetic construct
  source 1..675: mol_type = other DNA
atgaaaacaattatagctctatcatatatattctgcctggtgtttgcgaccaacttatgcccgtttggtgaggtgttcaacgcaacccgttttgcctctgtttacgcgtggaatcgtaagcgcatcagcaattgcgttgcggactacagcgtcttgtacaacagcgcatcctttagcacctttaaatgctacggcgtaagcccgactaagctgaatgatctgtgctttaccaacgtctatgcggactcgtttgttatccgtggtgatgaagttcgtcagatcgctccgggtcaaaccggcaaaatcgcggactataactacaaactgccggatgacttcacaggttgtgttattgcgtggaattccaacaaccttgatagcaaggtgggcggcaactacaattatctgtacagattgttccgcaagtctaacctgaaaccgttcgagcgtgacatttccacggaaatttatcaagctggctctaccccgtgtaatggcgtggaaggcttcaactgctacttcccactgcagagctatggtttccaaccgacgaacggtgttggctatcagccgtatcgcgtggtggtgctgagcttcgagttgctccatgcccctgctaccgtttgtggtccgaaaaagggttcaggtcatcaccaccatcaccaccaccattaa